SEQUENCE LISTING (39 sequences)

SEQ ID NO: 1 - DNA, 35 bp, Artificial Sequence
  Synthetic oligonucleotide
cccccccctcgagcttgcaatgatgtcgtaatttg

SEQ ID NO: 2 - DNA, 36 bp, Artificial Sequence
  Synthetic oligonucleotide
ccccaagcttaaaaatttcttgctcagtaagaattt

SEQ ID NO: 3 - DNA, 35 bp, Artificial Sequence
  Synthetic oligonucleotide
cccccccgaattccttgcaatgatgtcgtaatttg

SEQ ID NO: 4 - DNA, 36 bp, Artificial Sequence
  Synthetic oligonucleotide
ccccggatccaaaaatttcttgctcagtaagaattt

SEQ ID NO: 5 - DNA, 32 bp, Artificial Sequence
  Synthetic oligonucleotide
atcgatccgcggatgctggggggaggggggat

SEQ ID NO: 6 - DNA, 54 bp, Artificial Sequence
  Synthetic oligonucleotide
acgtgttaacgcggccgcagtctacttttgaaactctgccccttgtctcctaga

SEQ ID NO: 7 - DNA, 37 bp, Artificial Sequence
  Synthetic oligonucleotide
atcgatacgcgtctaaggaccagcttctttgggagag

SEQ ID NO: 8 - DNA, 32 bp, Artificial Sequence
  Synthetic oligonucleotide
atcgatggtaccgatcttcgggctctgccccg

SEQ ID NO: 9 - DNA, 110 bp, Artificial Sequence
  Synthetic oligonucleotide
gaaaatgactttgccacgcttagcatgtgacgaggtggccgagtggttaaggcgatggactgctaatccattgtgctctgcacgcgtgggttcgaatcccatcctcgtcg

SEQ ID NO: 10 - DNA, 59 bp, Artificial Sequence
  Synthetic oligonucleotide
ggcctgggagacctggggacgctgtgacacttcaaacttccccagctctcccaggcccg

SEQ ID NO: 11 - DNA, 47 bp, Artificial Sequence
  Synthetic oligonucleotide
gcctgggagagctggggaattcaagagattccccagctctcccaggc

SEQ ID NO: 12 - DNA, 50 bp, Artificial Sequence
  Synthetic oligonucleotide
agtgtcaagtccaatctatgattcaagagatcatagattggacttgacac

SEQ ID NO: 13 - DNA, 36 bp, Artificial Sequence
  Synthetic oligonucleotide
gggtctagaatggacaaggattgtgaaatgaaacgc

SEQ ID NO: 14 - DNA, 35 bp, Artificial Sequence
  Synthetic oligonucleotide
ggggaattccgtacgtcagtttcggccagcaggcg

SEQ ID NO: 15 - DNA, 60 bp, Artificial Sequence
  Synthetic oligonucleotide
atgcgccggcatcgatgaaaatgactttgccacgcttagcatgtgacgaggtggccgagt

SEQ ID NO: 16 - DNA, 60 bp, Artificial Sequence
  Synthetic oligonucleotide
atgcggcgccatttaaataaaaaagtgtcaagtccaatctatgatctcttgaatcataga

SEQ ID NO: 17 - DNA, 21 bp, Artificial Sequence
  Synthetic oligonucleotide
gcggagacagcgacgaagagc

SEQ ID NO: 18 - DNA, 21 bp, Artificial Sequence
  Synthetic oligonucleotide
gcctgtgcctcttcagctacc

SEQ ID NO: 19 - DNA, 21 bp, Artificial Sequence
  Synthetic oligonucleotide
catctcctatggcaggaagaa

SEQ ID NO: 20 - DNA, 26 bp, Artificial Sequence
  Synthetic oligonucleotide
cgtcagcgtcattgacgctgcgccca

SEQ ID NO: 21 - DNA, 26 bp, Artificial Sequence
  Synthetic oligonucleotide
gagtgcttttcgaaaactcatcagaa

SEQ ID NO: 22 - DNA, 21 bp, Artificial Sequence
  Synthetic oligonucleotide
ccagagagctcccaggctcag

SEQ ID NO: 23 - DNA, 20 bp, Artificial Sequence
  Synthetic oligonucleotide
tatggaacgcttctcgaatt

SEQ ID NO: 24 - DNA, 23 bp, Artificial Sequence
  Synthetic oligonucleotide
aaagtgtcaagtccaatctatga

SEQ ID NO: 25 - DNA, 36 bp, Artificial Sequence
  Synthetic oligonucleotide
gtgtcaagtttcgtccacacggactcatcagcaatc

SEQ ID NO: 26 - DNA, 20 bp, Artificial Sequence
  Synthetic oligonucleotide
agaacagatactacacttga

SEQ ID NO: 27 - DNA, 50 bp, Artificial Sequence
  Synthetic oligonucleotide
gtcgtatccagtgcagggtccgaggtattcgcactggatacgacagcgga

SEQ ID NO: 28 - DNA, 26 bp, Artificial Sequence
  Synthetic oligonucleotide
tcgcactggatacgacagcggagaca

SEQ ID NO: 29 - DNA, 18 bp, Artificial Sequence
  Synthetic oligonucleotide
gcctcttcgtcgctgtct

SEQ ID NO: 30 - DNA, 16 bp, Artificial Sequence
  Synthetic oligonucleotide
gtgcagggtccgaggt

SEQ ID NO: 31 - DNA, 25 bp, Artificial Sequence
  Synthetic oligonucleotide
atctgagcctgggagctctctggct

SEQ ID NO: 32 - DNA, 24 bp, Artificial Sequence
  Synthetic oligonucleotide
tgcgtcttactctgttctcagcga

SEQ ID NO: 33 - DNA, 24 bp, Artificial Sequence
  Synthetic oligonucleotide
cgtcaaccttctgtaccagcttac

SEQ ID NO: 34 - DNA, 26 bp, Artificial Sequence
  Synthetic oligonucleotide
gctcgcttcggcagcacatatactaa

SEQ ID NO: 35 - DNA, 24 bp, Artificial Sequence
  Synthetic oligonucleotide
acgaatttgcgtgtcatccttgcg

SEQ ID NO: 36 - DNA, 21 bp, Artificial Sequence
  Synthetic oligonucleotide
ttcattacacctgcagctctc

SEQ ID NO: 37 - DNA, 23 bp, Artificial Sequence
  Synthetic oligonucleotide
cctgttagagctactgcaattat

SEQ ID NO: 38 - DNA, 20 bp, Artificial Sequence
  Synthetic oligonucleotide
cgctctctgctcctcctgtt

SEQ ID NO: 39 - DNA, 20 bp, Artificial Sequence
  Synthetic oligonucleotide
ccatggtgtctgagcgatgt

(19) United States
(12) Patent Application Publication
(10) Pub. No.: US 2016/0289681 A1
(43) Pub. Date: Oct. 6, 2016

(54) RNA-BASED HIV INHIBITORS

(71) Applicant: City of Hope, Duarte, CA (US)

(72) Inventors: John Rossi, Azusa, CA (US); David DiGiusto, Claremont, CA (US); Janet Chung, Arlington, MA (US); Lisa Scherer, Monrovia, CA (US)

(21) Appl. No.: 15/073,481

(22) Filed: Mar. 17, 2016

Related U.S. Application Data
(63) Continuation of application No. PCT/US2014/056384, filed on Sep. 18, 2014.
(60) Provisional application No. 61/879,617, filed on Sep. 18, 2013.

(51) Int. Cl. C12N 15/113; A61K 48/00; C12N 15/86; C12N 7/00

(52) U.S. Cl. CPC C12N 15/1132; C12N 15/1138; C12N 7/00; A61K 48/005; C12N 15/86; C12N 2310/14; C12N 2310/531; C12N 2320/31; C12N 2310/13; C12N 2310/51; C12N 2740/15043; C12N 2830/20

(57) ABSTRACT

Provided herein are, inter alia, antiviral recombinant nucleic acid compositions and methods of using the same. The recombinant nucleic acid compositions include nucleic acids encoding antiviral polycistronic RNAs, which are capable of inhibiting viral replication. The antiviral recombinant nucleic acid compositions provided herein are therefore particularly useful for therapeutic applications such as combinational HIV-1 gene therapy.

### CROSS-REFERENCE TO RELATED APPLICATIONS

This application is a continuation of PCT Application No. PCT/US2014/056384 filed Sep. 18, 2014, which claims the benefit of U.S. Provisional Application No. 61/879,617 filed Sep. 18, 2013, which are hereby incorporated in their entirety and for all purposes.

### STATEMENT AS TO RIGHTS TO INVENTIONS MADE UNDER FEDERALLY SPONSORED RESEARCH AND DEVELOPMENT

This invention was made with government support under AI 42552, AI29329 and HL07470 awarded by the National Institutes of Health. The Government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 48440-533N01US_SequenceListing.TXT, created Sep. 18, 2014, 8,445 bytes, machine format IBM-PC, MS-Windows operating system, is hereby incorporated herein by reference in its entirety and for all purposes.

### BACKGROUND OF THE INVENTION

HIV gene expression is a highly regulated process that involves alternative splicing from the full-length 9-kb RNA genome to generate various viral proteins. Early in the replication cycle, transcription elongation is inefficient despite having a functional viral promoter, resulting in short or early terminated transcripts until the early regulatory protein Tat is made. Tat functions to vastly increase transcription through the transactivation of RNA Pol II polymerase from the viral promoter (Garber, M. E., and Jones, K. A., Curr Opin Immunol., 11, 460-465 (1999); Kao, S. Y. et al., Nature, 330, 489-493 (1987); Laspia, M. F., Wendel, P., and Mathews, M. B., J Mol Biol., 232, 732-746 (1993); Marciniak, R. A., and Sharp, P. A., Embo J., 10, 4189-4196 (1991); Toohey, M. G., and Jones, K. A., Genes Dev., 3, 265-282 (1989)) via interaction with the transactivation response (TAR) element in the 5′ untranslated region (UTR) of the viral RNAs (Kao, S. Y. et al., Nature, 330, 489-493 (1987)). Once Tat is available, transcription becomes processive and multiply spliced transcripts are produced that encode the other regulatory proteins, including Rev. Rev facilitates the export of partially spliced and unspliced transcripts to the cytoplasm for translation into late structural proteins by interactions with the Rev response element (RRE) present on these transcripts (Cullen, B. R., and Malim, M. H., Trends Biochem Sci, 16, 346-350 (1991); Felber, B. K. et al., Proc Natl Acad Sci USA, 86, 1495-1499 (1989); Krug, R. M., Curr Opin Cell Biol, 5, 944-949 (1993)). In addition to their known functions in the nucleus, Tat and Rev exhibit nucleolar-localizing properties with poorly understood functions, hypothesized as part of the transport mechanism or temporal storage [Tat (Li, Y. P., J Virol., 71, 4098-4102 (1997); Luznik, L. et al., J Clin Invest., 95, 328-332 (1995); Ruben, S. et al., J Virol., 63, 1-8 (1989); Siomi, H. et al., J Virol., 64, 1803-1807 (1990); Stauber, R. H., and Pavlakis, G. N., Virology, 252, 126-136 (1998); Rev (Dundr, M. et al., J Cell Sci., 108, 2811-2823 (1995); Nosaka, T. et al., Exp Cell Res., 209, 89-102 (1993)]. Similarly, full-length and partially spliced HIV transcripts have also been detected by electron microscopy in situ hybridization (Canto-Nogues, C. et al., Micron, 32, 579-589 (2001)). Taken together, these results suggest HIV-1 RNAs traffic through the nucleolus as part of the replication cycle. The importance of the nucleolus during viral replication could be more universal as transcription and replication of Boma disease virus (a negative strand RNA virus) occurs in the nucleolus (Pyper, J. M., Clements, J. E., and Zink, M. C., J Virol., 72, 7697-7702 (1998)). Interestingly, env RNAs from the human T-lymphotropic virus were also detected in the nucleolus (Kalland, K. H. et al., New Biol., 3, 389-397 (1991)).

To investigate whether the nucleolus plays an important role in HIV replication, nucleolar-localizing TAR and RBE RNA decoys (U16TAR and U16RBE, respectively) that function to trap HIV-1 Tat and Rev proteins inside the nucleolus, and a RNA ribozyme that targets a conserved U5 region of HIV-1 RNA (U16U5RZ) were created by substituting these anti-HIV small RNAs for the apical loop of the C/D box U16 small nucleolar RNA (snoRNA) (FIG. 1). The U16 chimeric RNAs were shown to localize in the nucleolus and each was shown to have strong anti-HIV-1 activity (Michienzi, A. et al., Proc Natl Acad Sci USA, 99, 14047-14052 (2002); Michienzi, A. et al., AIDS Res Ther., 3, 13 (2006; Unwalla, H. J. et al., Mol Ther., 16, 1113-1119 (2008)). It was also noted in these studies that the nucleolar-localizing TAR RNA decoy was a far more potent inhibitor than a nuclear-localizing counterpart. These results strengthened the importance of the nucleolar trafficking of Tat during viral replication (Michienzi, A. et al., Proc Natl Acad Sci USA, 99, 14047-14052 (2002)). Taken together, these findings suggest nucleolar trapping could be a novel avenue for developing anti-HIV therapeutics and support the important functional role of Tat and Rev nucleolar localization in viral replication (Michienzi, A. et al., Proc Natl Acad Sci USA, 99, 14047-14052 (2002)).

Applicants previously chose the naturally occurring polycistronic miR-106b cluster located in intron 13 of the protein encoding MCM7 gene on chromosome 7 (termed MCM7) as the scaffold to co-express three anti-HIV small interfering RNAs (siRNAs) from a single RNA Pol II human U1 promoter (Aagaard, L. A. et al., Gene Ther., 15, 1536-1549 (2008)). These studies demonstrated efficient expression and processing of three siRNAs that target the common tat/rev exon (S1), rev (S2M), and tat (S3B), respectively, as miRNA mimics (Aagaard, L. A. et al., Gene Ther., 15, 1536-1549 (2008)). Although multiplexing siRNAs is an approach to mitigating viral escape mutants found with a single point mutation in the siRNA target site (Boden, D. et al., J Virol., 77, 11531-11535 (2003); Das, A. T. et al., J Virol., 78, 2601-2605 (2004); Sabariegos, R. et al., J Virol., 80, 571-577 (2006)), Applicants believe that it is also advantageous to explore the potential for combining different types of small RNA inhibitors to further reduced the likelihood of viral resistance and to exploit the potential synergy between small RNA agents within a single gene therapy construct (Li, M. J., Mol Ther., 8, 196-206 (2003)). The MCM7 platform offers additional advantages and flexibility over multiple small RNA agents expressed with constitutive independent Pol III promoters (e.g., (Li, M. J., Mol Ther., 8, 196-206 (2003)) by offering opportunities to engineer tissue specificity by proper promoter choice while reducing toxicity related to over-expression. Applicants previously demonstrated that the MCM7 platform could also be used for co-expression of the U16TAR RNA decoy by replacing the S3B subunit, as shown by the MCM7-S1/S2M/U16TAR construct (Aagaard, L. A. et al., Gene Ther., 15, 1536-1549 (2008)). The processing of U16TAR was shown to be independent of Drosha, implicating that the snoRNA was processed independently of the siRNAs via the C/D box small nucleolar ribonucleoprotein (snoRNP) processing pathway (Aagaard, L. A. et al., Gene Ther., 15, 1536-1549 (2008)). Here Applicants demonstrate that multiple small nucleolar RNAs can also be incorporated in this platform where they are effectively processed along with the siRNAs to provide a combinatorial, long-term inhibition of HIV-1 replication in CEM T-lymphocytes. The combinations of si/sno RNAs represent a new paradigm for combinatorial RNA-based gene therapy applications.

### BRIEF SUMMARY OF THE INVENTION

In one aspect, a recombinant nucleic acid encoding an antiviral polycistronic RNA is provided. The recombinant nucleic acid includes a first RNA promoter operably linked to: (i) a first antiviral RNA encoding sequence, (ii) a second antiviral RNA encoding sequence and a (iii) third antiviral RNA encoding sequence, wherein the first RNA promoter is a forward promoter.

In another aspect, a recombinant nucleic acid encoding an antiviral polycistronic RNA is provided. The recombinant nucleic acid includes a first RNA promoter operably linked to: (i) a first antiviral RNA encoding sequence, a second antiviral RNA encoding sequence and a third antiviral RNA encoding sequence; and (ii) a second RNA promoter operably linked to a viral entry inhibiting RNA encoding sequence.

In another aspect, a mammalian cell including a recombinant antiviral polycistronic RNA is provided. The recombinant antiviral polycistronic RNA includes (i) a first antiviral RNA, a second antiviral RNA and a third antiviral RNA; and (ii) a viral entry inhibiting RNA.

In another aspect, a kit including a recombinant antiviral polycistronic RNA is provided. The recombinant antiviral polycistronic RNA includes (i) a first antiviral RNA, a second antiviral RNA and a third antiviral RNA; and (ii) a viral entry inhibiting RNA.

In another aspect, a pharmaceutical composition including a pharmaceutically acceptable excipient and a recombinant viral particle including a recombinant nucleic acid as provided herein including embodiments thereof is provided.

In another aspect, a method of treating an infectious disease in a subject in need thereof is provided. The method includes administering to the subject a therapeutically effective amount of a recombinant viral particle including a recombinant nucleic acid as provided herein including embodiments thereof.

In another aspect, a method of inhibiting HIV replication in a patient is provided. The method includes administering to the patient a therapeutically effective amount of a recombinant viral particle including a recombinant nucleic acid as provided herein including embodiments thereof, thereby inhibiting HIV replication in the patient.

### BRIEF DESCRIPTION OF THE DRAWINGS

FIG. 1. Construction of small nucleolar anti-HIV RNAs. The C/D box U16 small nucleolar RNA (snoRNA) is used as a scaffold to construct nucleolar-localizing anti-HIV small RNAs. The conserved C/D box of the U16 snoRNA is sufficient for the nucleolar-localizing property with the apical loop replaced with various anti-HIV RNAs including the U5 targeting RNA ribozymes (U16U5RZ), the Rev binding element RNA decoy (U16RBE), and the transactivation response RNA decoy (U16TAR). Sequence legend: U16U5RZ (SEQ ID NO:21); U16RBE (SEQ ID NO:20); U16TAR (SEQ ID NO:22).

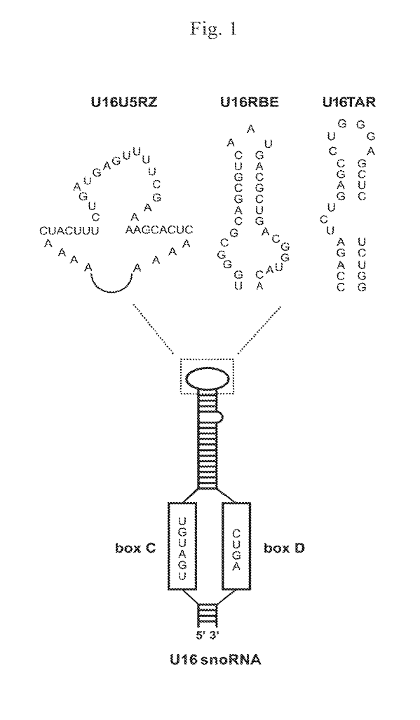

FIG. 2. Overiew of MCM7 intron-based lentiviral vectors. The name “MCM7” refers to a naturally occurring polycistronic miRNA cluster located in an intron of the MCM7 gene. Exons and intron of the MCM7 cassette are drawn as grey boxes and black lines, respectively, with splice donor and acceptors marked as “SD” and “SA”. Promoters are denoted by white boxes with the arrow indicating directionality, while the terminators are denoted by black boxes. shRNA, U16 snoRNA scaffold, and apical loop anti-HIV RNA insert are shown. (a) The MCM7 scaffold allows co-expression of three small RNAs from the single Pol II U1 promoter. S1, S2M, and S3B represent siRNAs targeting the common tat/rev exon, rev, and tat, respectively. U16U5RZ is a nucleolar-localizing ribozyme targeting a conserved U5 region present in all HIV transcripts. U16TAR is a nucleolar-localizing TAR RNA decoy. U16RBE is a nucleolar-localizing Rev binding element RNA decoy. (b) The MCM7 cassette with the U1-specific termination sequence (Ult) was cloned into the pHIV7-EGFP lentiviral vector in the forward orientation with respect to the CMV packaging promoter, denoted as “Forward-Ult”, while the cassette in the opposite orientation is denoted as “Reverse-Ult.” RRE, Rev response element; MCS, multiple cloning site; EGFP, enhanced green fluorescent protein; WPRE, woodchuck hepatitis virus post-transcription regulation element; ΔU3, deleted U3 region to generate a self-inactivating lentiviral vector after integration in targeted cells.

FIG. 3. Expression of the small RNAs in stably transduced CEM T-lymphocytes. CEM T-lymphocytes were transduced with lentiviruses containing MCM7 cassette in the forward orientation at an MOI of 50. About 20 μg of total RNA were loaded per lane and electrophoresed in an 8% polyacrylamide gel with 8M urea, blotted onto a nylon membrane, and hybridized with the corresponding ³²P-labelled probes. RNA prepared from untransduced cells and cells transduced with empty vector were used as negative controls. S1, S2M, and S3B siRNAs are approximately 21 nucleotides. The U16 snoRNA chimeras are approximately 132 nucleotides. U6 small nuclear RNA serves as a loading control.

FIG. 4. Anti-HIV activity of MCM7-based constructs. One million untransduced and stable CEM T-lymphocytes were challenged in triplicate with NL4-3 strain of HIV-1 at an MOI of 0.01 and culture supernatants were collected weekly for the HIV-1 p24 antigen ELISA to evaluate viral replication. The dashed line represents the low detection limit of the p24 assay. Three constructs, MCM7-S1/S2M/S3B, MCM7-S1/S2M/U16TAR, and MCM7-S1/U16U5RZ/U16TAR showed potent antiviral activity with almost no detectable viral load during the one-month challenge assay.

FIG. 5. In vitro viral mediated selection of transduced CEM T-lymphocytes with optimal anti-HIV gene expression. (a) Total RNA was extracted from CEM T-lymphocytes infected with HIV-1 at designated time points (D0, D14, and D28) to evaluate RNA expression. The S1 siRNA expression was evaluated by qRT-PCR and normalized by the internal control U6 small nuclear RNA. (b) Total RNA was extracted from CEM T-lymphocytes infected with HIV-1 at designated time points (D0, D14, and D28) to evaluate RNA expression. The U16 TAR RNA decoy expression was evaluated by qRT-PCR and normalized by the internal control U6 small nuclear RNA. *p<0.05, **p<0.01, ***p<0.001, difference compared to uninfected control (D0).

FIG. 6. Anti-HIV RNA expression correlates with HIV-1 pNL4-3 luciferase knockdown activity. Left panel: HEK293 cells were transfected at 80% confluency in 48-well plate with 20 ng of replication-deficient pNL4-3 proviral DNA harboring the firefly luciferase gene (pNL4-3.Luc.R-.E, NIH AIDS reagent and repository) and 3.17×10⁻² pmole of plasmids with the anti-HIV RNAs driven either by the U1 or U6 promoter (in 300 ng total mass with pBluescript plasmid) complexed with Lipofectamine 2000 (Invitrogen). The pNL4-3 luciferase construct maintains targets for each of the small RNAs in all the transcripts, both spliced and unspliced and therefore luciferase readouts can be utilized as quantitative readouts of viral inhibition. Firefly luciferase output was normalized to the internal control Renilla luciferase to account for differences in transfection efficiency. Data presented consist of two independent experiments. **p<0.01. Right panel: HEK293 cells were transiently transfected at 90% confluency in 6-well plate with 0.334 pmole of either the MCM7 cassette containing three snoRNAs or single snoRNA expressed with U6 promoter (pTZ/U6-U16RBE, pHIV7-U6-U16U5RZ, or pTZ/U6-U16TAR), or their combinations, in total mass of 4 μg with pBluescript plasmid, complexed with Lipofectamine 2000 (Invitrogen). Total RNA was extracted 48 hours later with STAT-60 reagent according to manufacturer's instructions. About 10 μg of total RNA were loaded per lane and RNA was detected with ³²P-labelled probes as described in Material and Methods.

FIG. 7. Second generation RNA-based gene therapy constructs. Schematic MCM7 transgene expression is shown.

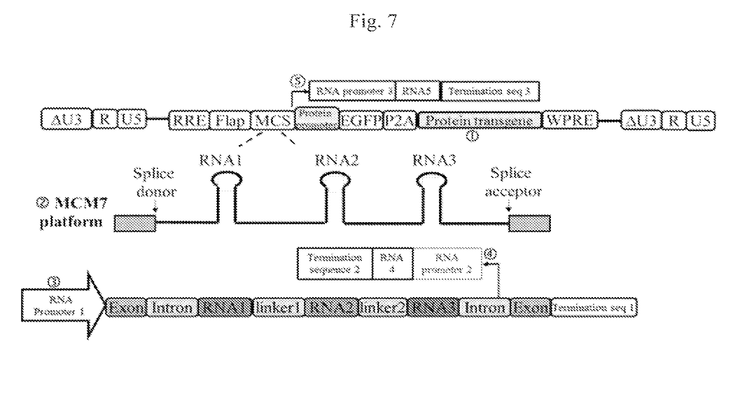

FIG. 8. Northern blotting experiment to investigate transgene expression level with cassette orientation. HEK 293 cells were transiently transfected with constructs carrying the RNA cassettes and total RNA extracted 48 hours post transfection. Left panel: Bifunctional siRNAs (CCR5-5) targeting UTR regions of CCR5 and HIV were expressed as a pre-miRNA (“NTS”) or as a shRNA (“19sh”). Right panel: siRNA against coding region of CCR5 (“CCR5-12sh”) was expressed as from the Pol III tRNA^{Ser} promoter in forward (“F”) or reverse (“R”) orientations. About 20 μg of total RNA was loaded per lane and electrophoresed in an 8% polyacrylamide gel with 8 M urea, blotted onto a nylon membrane, and hybridized with the corresponding ³²P-labeled probes. The expression cassette can produce mature siRNA sequences that are approximately 21 nucleotides. U2A small nuclear RNA serves as a loading control. In all cases, cassette in reverse orientation consistently gives more transgene expression.

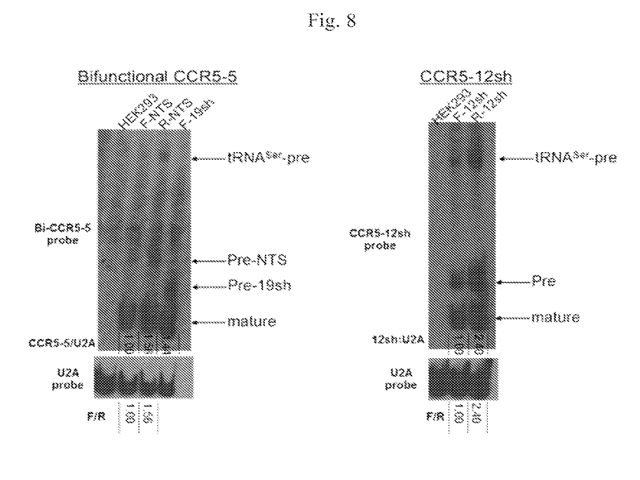

FIG. 9. Psi-check assay to monitor down-regulation of CCR5 and HIV UTR targets. In this experiment the target sequence is cloned in the 3′ UTR of the reporter Renilla luciferase gene and the fusion transcript is subject to gene silencing by RNA interference. The firefly luciferase reporter serves as a mean to normalize for differences in transfection efficiency. The ratio of Renilla and firefly luciferase expression provides a measure of gene silencing. In this context, bifunctionality siRNAs expressed as a pre-miRNA or as a shRNA are both capable of mediating HIV and CCR5 target knockdown.

FIG. 10. Effect of mature siRNA sequences and CCR5 surface expression. Applicants utilized the U373-MAGI-CCR5E cell line (obtained through the NIH AIDS Reagent Program, Division of AIDS, NIAID, NIH and as published in Vodicka M. A., Virology, 233:193-198, (1997)) that over-expresses CCR5 and transiently transfected Applicants' constructs to monitor (A) CCR5 expression and (B) CD4 expression by flow cytometry. Specific decrease in CCR5 expression was only observed with cells transfected with tRNASer-CCR5-12sh cassette.

FIG. 11. Northern analysis of anti-HIV small RNA expression in stably transduced CEM T lymphocytes. CEM T lymphocytes were transduced and sorted based on GFP expression. Northern analysis was performed to validate correct small RNA transgene expression and processing. RNA prepared from untransduced cells and cells transduced with empty vector were used as negative controls. 51, S2M, S3B, and CCR5-12sh siRNAs are approximately 21-nt. The U16 snoRNA chimeras are 132-nt. The small nuclear U2A RNA served as a loading control. Northern analysis is shown in the left panel, loading scheme is shown in the right panel.

FIG. 12. Anti-HIV activity of MCM7-based constructs. One million untransduced and stably transduced CEM T-cells were challenged with JR-FL strain of HIV-1 at MOI of 0.01 and culture supernatants were collected weekly for the HIV-1 p24 ELISA to evaluate viral replication. Second generation MCM7-based constructs potently inhibited viral replication with 3 to 5-log reduction of p24 production during the 42-day viral challenge.

FIG. 13. Overview of strategies to multiplex small RNAs with different classes of promoters. a) pHIV7 lentiviral with MGMT^{P140K} selectable marker (pLV). Applicants utilized a third generation HIV-1 based lentiviral vector (pHIV7) with EGFP marker to label gene modified cells. The chemical resistance MGMT^{P140K} gene is co-expressed with EGFP with a self-cleaving P2A peptide with the CMV promoter. b) First generation lentiviral vector (FGLV) with small RNAs expressed from independent RNA Pol III promoters. In the first generation lentiviral vector, each antiviral small RNA transgene is expressed independently from RNA Pol III promoter. c) Second generation lentiviral vector (SGLV) with small RNAs expressed in the MCM7 polycistronic platform with single RNA Pol II promoter. The naturally occurring miRNA cluster in the intron of the human MCM7 gene was engineered to co-express different classes of antiviral small RNAs with single Pol II U1 promoter for ubiquitous transgene expression in all hematopoietic lineages. Independent Pol III RNA cassettes can be incorporated for expression of up to five small RNAs. The tRNA^{Ser}-CCR5sh cassette was incorporated into the 3′ intron of MCM7 in both orientations (“F” Forward and “R” Reverse) with respect to the parental U1 promoter. The U6-U16TAR cassette was cloned downstream of the U1 termination signal. Promoters: CMV, cytomegalovirus promoter and enhancer sequence (Pol II); U6, human small nuclear U6 promoter (Pol III); VA1, adenoviral promoter (Pol III); U1, human small nuclear U1 promoter (Pol II); tRNA^{Ser}, human transfer RNA Serine promoter (Pol III). Small RNA transgenes: 51, tat/rev siRNA; S2M; rev siRNA; S3B tat siRNA; CCR5sh, CCR5-targeting shRNA; U16TAR, nucleolar TAR RNA decoy; U16USRZ, nucleolar US-targeting ribozyme; CCR5RZ, CCR5-targeting ribozyme.

FIG. 14. Biological activity of tRNA^{Ser}-CCR5sh cassette. a) Potent CCR5 knockdown in U373-MAGI-CCR5E cells. Plasmid with only the tRNA^{Ser} promoter sequence (solid black line) or with the tRNA^{Ser}-CCR5sh cassette (dashed grey line) was transiently transfected into CCR5 over-expressing U373-MAGI-CCR5E cells with knockdown estimated by flow cytometry 72 hours later. Potent and specific down-regulation of CCR5 surface expression was only observed with the construct containing the CCR5sh RNA. b) Potent CCR5 transcript degradation in CD34-derived macrophages. Adult CD34+ HSPCs were transduced with the indicated lentiviral vectors, sorted based on EGFP expression, then differentiated into macrophages as described in Methods and Materials. CCR5 transcript knockdown was measured by qRT-PCR with normalization with GAPDH housekeeping gene then to the untransduced control. tRNA^{Ser}-CCR5sh cassette in the context of MCM7 platform induced potent silencing in gene modified macrophages.

FIG. 15. Optimization of the tRNA^{Ser}-CCR5sh cassette expression in the MCM7 platform. a) Orientation dependence of the tRNA^{Ser}-CCR5sh cassette in MCM7. The tRNA^{Ser}-CCR5sh cassette was cloned in 3′ intron of MCM7 either in the forward (SGLV3) or reverse (SGLV4) orientation, then transiently transfected into HEK 293 cells to evaluate transgene expression and processing by Northern blotting. Northern blotting distinguishes products in various steps of processing due to difference in size [tRNA^{Ser}-CCR5sh fusion transcript (130-140 nt) that requires tRNase Z processing; shRNA (˜50-60 nt) that requires Dicer processing; siRNA (20-23 nt) represents the completely processed mature siRNA that is capable of mediating silencing]. In this case, the probe detected the guide strand that mediates CCR5 silencing. Northern blotting demonstrated tRNA^{Ser}-shRNA cassette is efficiently processed by the RNA interference pathway as the mature siRNA is the predominate product. Furthermore, SGLV4 gives 2.4-fold enhancement in transgene expression in comparison with the opposite orientation after normalization with the loading control U2A RNA. b) Placement dependence of the tRNA^{Ser}-CCR5sh cassette. Placement of tRNA^{Ser}-CCR5sh cassette in the lentiviral vector dramatically affects transgene expression in sorted stably expressing CEM T lymphocytes. Although the CCR5sh cassette is driven independently from the tRNA^{Ser} promoter, the expression was much lower in the context of MCM7 platform (“inside of MCM7”) compared to as a separate entity (“outside MCM7”). In the latter scenario, over-expression is evident by the presence of unprocessed products (i.e., bands representing tRNA^{Ser}-CCR5sh fusion transcript and shRNA).

FIG. 16. Northern blot of stably expressing CEM T lymphocytes demonstrates efficient processing and expression of small RNAs. CEM T lymphocytes were transduced with indicated lentiviruses carrying the combinational vectors then sorted by EGFP expression to create stably expressing cell lines. Small RNA transgenes were detected by P³² labeled probes. U2A small nuclear RNA serves as a loading control. 51, S2M, S3B, CCR5sh represent 20-23 nt fully processed siRNAs. U16U5RZ and U16TAR are U16 snoRNA chimeras that are approximately 132 nucleotides. CCR5RZ is approximately 230 nucleotides.

FIG. 17. Intracellular HIV-1 staining demonstrates potent antiviral protection for macrophages derived from gene modified CD34+ HSPCs. Macrophages differentiated from adult CD34+ HSPCs transduced with indicated lentiviral vectors (a) uninfected; b) untransduced; c) FGLV; d) SGLV1; e) SGLV2; f) SGLV4; g) SGLV5; h) SGLV6; i) SGLV 7) were challenged with HIV-1 Bal at MOI=0.01. Viral infection was monitored by intracellular staining by flow cytometry with an antibody specific to HIV-1 core proteins. Data from 18 days post infections are shown. Background signal for intracellular staining was established with an uninfected control with identical culture and staining protocol. Intracellular staining showed a high degree of infection in unprotected macrophages, with some constructs with intermediate protection while differentiating some with excellent protection.

FIG. 18. Kinetics of R5 tropic HIV-1 Bal infection in adult CD34+ HSPC derived macrophages monitored by intracellular HIV staining. Kinetics of HIV-1 Bal infection in macrophages differentiated from gene modified HSPCs were followed by intracellular HIV staining for a total of 42 days to evaluate long term protection and viral breakthrough. Over-expression of therapeutic small RNAs with independent Pol III promoters (FGLV) provided potent protection for up to 28 days but eventual breakthrough. In the long term, SGLV2 provided the longest protection with the incorporation of tRNA^{Ser}-CCR5sh (SGLV4) and U6-U16TAR (SGLV7) cassettes less optimal.

FIG. 19. In vitro CFU assay to identify potential vector toxicity on hematopoietic potential. Transduced CD34+ HSPCs were sorted on CD34+/EGFP expression after expansion with SR1. A total of 500 sorted cells per sample were plated on methylcellulose medium in triplicate with number of colonies counted 12 to 13 days later. The absolute number of CFUs was normalized to the respective donor to account for differences in hematopoietic potential in donor viability. Result represented data from at least two independent donors and significant results were shown. * p<0.05, *** p<0.001, **** p<0.0001.

FIG. 20. In vivo drug selection enhances the frequency of gene modified cells in the bone marrow and spleen of humanized NSG mice expressing MGMT^{P140K}. Analysis of NSG mice transplanted with gene modified HSPCs expressing MGMT^{P140K} and treated with two or three doses of O⁶-BG/BCNU as described in text. Each mouse received 1×10⁶ CD34+ HSPCs following transduction at the date of transplantation and 20 μg Fc/IL7 protein for 11 weeks. (a) Frequency of CD45+ cells in the bone marrow of treated mice. (b) Frequency of CD45+/GFP+ cells in the bone marrow of treated mice. (c) Frequency of CD45+ cells in the spleens of treated mice. (d) Frequency of CD45+/GFP+ cells in the spleen of treated mice. (e) Frequency of CD3+/CD4+/GFP+ cells in the spleen of treated mice (gated on CD45+ population). (f) Frequency of CD14+/CD4+/GFP+ cells in the spleen of treated mice (gated on CD45+ population). **p<0.01, ***p<0.001, **** p<0.0001.

FIG. 21. Serum viremia in mice infected with HIV-1_{Bal}. Mice were transplanted with CD34+ HSPC transduced as described in text and infected with HIV-1Bal at 11 weeks after transplant. UTDX=untransduced controls, TDX transduced with indicated vector. Mice analyzed for FGLV UTDX N=7, TDX N=6 (left panel); for SGLV2, UTDX N=7, TDX N=8 (mid panel); for SGLV4 UTDX N=7, TDX N=3 (right panel).

FIG. 22. Levels of engraftment of cells following HIV challenge of humanized NSG mice. A) Overall CD3+/CD4+ T-cell levels among the CD45+ human cells in the spleen of humanized NSG mice 6 weeks after saline or HIV-1_{Bal} challenge. B) Level of CD45+/GFP+ cells in the same animals. C) Level of CD3+/CD4+/GFP+ T-cells among the CD45+ cells in same animals. D) Level of CD4+/CD14+/GFP+ monocytes among the CD45+ cells in same animals.

### DETAILED DESCRIPTION OF THE INVENTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, and 2-O-methyl ribonucleotides.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The term “plasmid” refers to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The term “promoter” or “regulatory element” refers to a region or sequence determinants located upstream or downstream from the start of transcription and which are involved in recognition and binding of RNA polymerase and other proteins to initiate transcription. Promoters need not be of viral origin, for example, mammalian cellular promoters, such as the polymerase II promoter U1 and polymerase III promoter tRNA^{Ser} may be used in the present invention.

A “siRNA,” “small interfering RNA,” “small RNA,” or “RNAi” as provided herein refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi refers to a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length. Non-limiting examples of siRNAs include ribozymes, RNA decoys, short hairpin RNAs (shRNA), micro RNAs (miRNA) and small nucleolar RNAs (snoRNA).

The term “antiviral RNA” as provided herein refers to an RNA that is capable of inhibiting the activity (e.g., transcription, translation, replication, infectivity) of a virus. In embodiments, the antiviral RNA binds to a target viral nucleic and reduces transcription of the target viral nucleic acid or reduces the translation of the target viral nucleic acid (e.g. mRNA) or alters transcript splicing. In embodiments, the antiviral RNA is a nucleic acid that is capable of binding (e.g. hybridizing) to a target viral nucleic acid (e.g. an Rev RNA) and reducing translation of the target viral nucleic acid. The target viral nucleic acid is or includes one or more target nucleic acid sequences to which the antiviral RNA binds (e.g. hybridizes). In embodiments, the antiviral RNA is or includes a sequence that is capable of hybridizing to at least a portion of a target viral nucleic acid at a target viral nucleic acid sequence. Non-limiting examples of an antiviral RNA include siRNAs, ribozymes, RNA decoys, snoRNAs and shRNAs.

A “polycistronic RNA” as provided herein refers to an RNA sequence including more than one (e.g., 2, 3, 4, 5, 6, 7) open reading frame (nucleic acid sequence encoding a polypeptide or an antiviral RNA). A polycistronic RNA as provided herein may include one promoter controlling the expression of all open reading frames encoded by the polycistronic RNA. In embodiments, the polycistronic RNA includes more than one promoter and one or more of the open reading frames included in the polycistronic RNA are expressed by an independent promoter.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For specific proteins described herein (e.g., CXCR4, CCR5, TNPO3, C46 fusion inhibitor, RevM10), the named protein includes any of the protein's naturally occurring forms, or variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment or homolog thereof.

A “MCM7 gene” as referred to herein includes any of the recombinant or naturally-occurring forms of the gene encoding DNA replication licensing factor MCM7 or variants or homologs thereof that maintain MCM7 protein activity (e.g. within at least 50%, 80%, 90%, 95%, 96%, 97%, 98%, 99% or 100% activity compared to MCM7). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MCM7 polypeptide. In embodiments, the MCM7 gene is substantially identical to the nucleic acid identified by the NCBI reference number Gene ID: 4176 or a variant or homolog having substantial identity thereto.

“CXCR4” or “CXCR4 gene” as referred to herein includes any of the recombinant or naturally-occurring forms of the gene encoding the C—X—C chemokine receptor type 4 or variants or homologs thereof that maintain CXCR4 protein activity (e.g. within at least 50%, 80%, 90%, 95%, 96%, 97%, 98%, 99% or 100% activity compared to CXCR4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CXCR4 polypeptide. In embodiments, the CXCR4 gene is substantially identical to the nucleic acid identified by the NCBI reference number GI: 56790928 or a variant or homolog having substantial identity thereto.

“CCR5” or “CCR5 gene” as referred to herein includes any of the recombinant or naturally-occurring forms of the gene encoding the C—C chemokine receptor type 5 or variants or homologs thereof that maintain CCR5 protein activity (e.g. within at least 50%, 80%, 90%, 95%, 96%, 97%, 98%, 99% or 100% activity compared to CCR5). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CCR5 polypeptide. In embodiments, the CCR5 gene is substantially identical to the nucleic acid identified by the NCBI reference number GI: 154091327 or a variant or homolog having substantial identity thereto.

“TNPO3” or “TNPO3 gene” as referred to herein includes any of the recombinant or naturally-occurring forms of the gene encoding the transportin-3 protein or variants or homologs thereof that maintain TNPO3 protein activity (e.g. within at least 50%, 80%, 90%, 95%, 96%, 97%, 98%, 99% or 100% activity compared to TNPO3). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TNPO3 polypeptide. In embodiments, the TNPO3 gene is substantially identical to the nucleic acid identified by the NCBI reference number GI: 300934784 or a variant or homolog having substantial identity thereto.

“Tat” or “Tat gene” as referred to herein includes any of the recombinant or naturally-occurring forms of the gene encoding the HIV-1 trans-activator of transcription or variants or homologs thereof that maintain Tat protein activity (e.g. within at least 50%, 80%, 90%, 95%, 96%, 97%, 98%, 99% or 100% activity compared to Tat). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Tat polypeptide. In embodiments, the Tat gene is substantially identical to the nucleic acid identified by the NCBI reference number GI: 1229009 or a variant or homolog having substantial identity thereto.

“Rev” or “Rev gene” as referred to herein includes any of the recombinant or naturally-occurring forms of the gene encoding the regulator of expression of virion proteins or variants or homologs thereof that maintain Rev protein activity (e.g. within at least 50%, 80%, 90%, 95%, 96%, 97%, 98%, 99% or 100% activity compared to Rev). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Rev polypeptide. In embodiments, the Rev gene is substantially identical to the nucleic acid identified by the NCBI reference number GI: 9629359 or a variant or homolog having substantial identity thereto.

A “Rev M10 protein” as referred to herein is a dominant negative mutant of a Rev protein or homolog thereof. In embodiments, the Rev M10 protein is substantially identical to the protein identified by the NCBI reference number ID 238635393.

The term “sample” includes sections of tissues such as biopsy and autopsy samples, and frozen sections taken for histological purposes. Such samples include blood and blood fractions or products (e.g., bone marrow, serum, plasma, platelets, red blood cells, and the like), sputum, tissue, cultured cells (e.g., primary cultures, explants, and transformed cells), stool, urine, other biological fluids (e.g., prostatic fluid, gastric fluid, intestinal fluid, renal fluid, lung fluid, cerebrospinal fluid, and the like), etc. A sample is typically obtained from a “subject” such as a eukaryotic organism, most preferably a mammal such as a primate, e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As used herein, the term “infectious disease” refers to a disease or condition related to the presence of an organism (the agent or infectious agent) within or contacting the subject or patient. Examples include a bacterium, fungus, virus, or other microorganism. A “bacterial infectious disease” is an infectious disease wherein the organism is a bacterium. A “viral infectious disease” is an infectious disease wherein the organism is a virus.

The term “associated” or “associated with” as used herein to describe a disease (e.g. an infectious disease) means that the disease (e.g. HIV infection) is caused by, or a symptom of the disease is caused by, or a symptom of the disease is caused by a virus (e.g., HIV).

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The terms “prevent,” “preventing” or “prevention,” and other grammatical equivalents as used herein, include to keep from developing, occur, hinder or avert a disease or condition symptoms as well as to decrease the occurrence of symptoms. The prevention may be complete (i.e., no detectable symptoms) or partial, so that fewer symptoms are observed than would likely occur absent treatment. The terms further include a prophylactic benefit. For a disease or condition to be prevented, the compositions may be administered to a patient at risk of developing a particular disease (e.g. hematological disease), or to a patient reporting one or more of the physiological symptoms of a disease, even though a diagnosis of this disease may not have been made.

Where combination treatments are contemplated, it is not intended that the agents (i.e. viral expression vectors, recombinant viral particles) described herein be limited by the particular nature of the combination. For example, the agents described herein may be administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein (e.g. Tat, Rev) relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Patient” or “subject in need thereof” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals, bovines, rats, mice, dogs, monkeys, goat, sheep, cows, deer, and other non-mammalian animals. In embodiments, a patient is human.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. Contacting may include allowing two species to react, interact, or physically touch, wherein the two species may be a recombinant viral particle as described herein and a cell.

As defined herein, the term “inhibition”, “inhibit”, “inhibiting” and the like in reference to an siRNA or protein-inhibitor interaction means negatively affecting (e.g., decreasing) the activity or function of the protein (e.g. decreasing gene transcription or translation) relative to the activity or function of the protein in the absence of the inhibitor. In embodiments, inhibition refers to reduction of a disease or symptoms of disease (e.g., HIV infection). In embodiments, inhibition refers to a reduction in the activity of a signal transduction pathway or signaling pathway (e.g. reduction of viral replication). Thus, inhibition includes, at least in part, partially or totally blocking stimulation, decreasing, preventing, or delaying activation, or inactivating, desensitizing, or down-regulating transcription, translation, signal transduction or enzymatic activity or the amount of a protein (e.g. a viral protein or a cellular protein). In embodiments, inhibition refers to inhibition of Tat. In embodiments, inhibition refers to inhibition of Rev. In embodiments, inhibition refers to inhibition of CCR5. In embodiments, inhibition refers to inhibition of CXCR4.

The terms “inhibitor,” “repressor” or “antagonist” or “downregulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. An “inhibitor” is a siRNA, (e.g., shRNA, miRNA, snoRNA, RNA decoy, ribozyme), compound or small molecule that inhibits viral infection (e.g., replication) e.g., by binding, partially or totally blocking stimulation, decrease, prevent, or delay activation, or inactivate, desensitize, or down-regulate signal transduction, gene expression or enzymatic activity necessary for protein activity. Inhibition as provided herein may also include decreasing or blocking a protein activity (e.g., activation of viral transcription) by expressing a mutant form of said protein thereby decreasing or blocking its activity.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The term “aberrant” as used herein refers to different from normal. When used to described enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

### Recombinant Nucleic Acids

Provided herein are, inter alia, antiviral recombinant nucleic acid compositions and methods of using the same. The recombinant nucleic acid compositions include nucleic acids encoding antiviral polycistronic RNAs, which are capable of inhibiting the activity of viral proteins (e.g., Tat, Rev) as well as the expression of cellular proteins (e.g., CCR5) utilized by the virus during its lifecycle. The antiviral recombinant nucleic acid compositions provided herein are therefore particularly useful for therapeutic applications such as combinational HIV-1 gene therapy.

The recombinant nucleic acids provided herein may encode a plurality of antiviral RNAs (e.g., siRNA, miRNA, shRNA, snoRNA). Thus, in one aspect, a recombinant nucleic acid encoding an antiviral polycistronic RNA is provided. The recombinant nucleic acid includes a first RNA promoter operably linked to: (i) a first antiviral RNA encoding sequence, (ii) a second antiviral RNA encoding sequence and a (iii) third antiviral RNA encoding sequence, wherein the first RNA promoter is a forward promoter.

An RNA promoter as provided herein refers to a nucleic acid sequence located upstream or downstream from the start of transcription of an RNA (e.g., siRNA, miRNA, shRNA, snoRNA). The RNA promoter provided herein may be a forward promoter or a reverse promoter. Where the RNA promoter is a forward promoter, the RNA polymerase synthesizes RNA from said promoter using the DNA antisense strand as template. Where the RNA promoter is a reverse promoter, the RNA polymerase synthesizes RNA using the DNA sense strand as template. The DNA sense strand corresponds to the mRNA strand or coding strand, whereas the DNA antisense strand corresponds to the non-coding strand, which is complementary to the mRNA. As provided herein the RNA promoter is involved in recognition and binding of RNA polymerase and other proteins to initiate transcription of the RNA. The RNA promoters contemplated for the present invention including embodiments thereof may be polymerase II or polymerase III promoters. Non-limiting examples of RNA promoters include polymerase II promoters such as the U1 promoter, the human elongation factor-1 alpha (EF-1 alpha) promoter, the cytomegalovirus (CMV) promoter, the human ubiquitin promoter, and the spleen focus-forming virus (SFFV) promoter; and the polymerase III promoters such as the U6 promoter, the H1 promoter, the tRNA^{Lys} promoter, the tRNA^{Ser} promoter and the tRNA^{Arg} promoter. Thus, in embodiments, the first RNA promoter is an RNA polymerase II promoter. In embodiments, the RNA polymerase II promoter is a small nuclear RNA (snRNA) promoter. In embodiments, the snRNA promoter is a U1 promoter.

In embodiments, the recombinant nucleic acid further includes a second RNA promoter operably linked to a viral entry inhibiting RNA encoding sequence, wherein the second RNA promoter is a reverse promoter. In embodiments, the second RNA promoter is downstream of the third antiviral RNA encoding sequence. In embodiments, the second RNA promoter is a polymerase III promoter. In embodiments, the RNA polymerase III promoter is a small nuclear RNA (snRNA) promoter. In embodiments, the snRNA promoter is a U6 promoter.

A viral entry inhibiting RNA encoding sequence as provided herein refers to a nucleic acid, which upon expression in a cell inhibits entry of a virus (e.g., HIV) into the cell. The viral entry inhibiting RNA may be a siRNA or a protein encoding RNA. Thus, in embodiments, the viral entry inhibiting RNA encoding sequence encodes a siRNA. In embodiments, the viral entry inhibiting RNA encoding sequence encodes a cellular receptor siRNA. In embodiments, the cellular receptor siRNA is a T cell receptor siRNA. In embodiments, the T cell receptor siRNA is a small hairpin (sh) RNA. In embodiments, the shRNA is a CCR5 shRNA. In embodiments, the shRNA is a CXCR4 shRNA. In embodiments, the viral entry inhibiting RNA encoding sequence encodes a nuclear receptor siRNA. In embodiments, the nuclear receptor siRNA is a transportin 3 (TNPO3) siRNA.

The recombinant nucleic acid provided herein may form part of a viral expression vector. A “viral vector” or “viral expression vector” is a viral-derived nucleic acid that is capable of transporting another nucleic acid into a cell. A viral vector is capable of directing expression (i.e. transcription and/or translation) of an RNA, a protein or proteins encoded by one or more genes carried by the vector when it is present in the appropriate environment. A viral expression vector may include a viral expression vector promoter (e.g., LTR) controlling transcription of an RNA, a protein or proteins encoded by one or more genes carried by the vector when it is present in the appropriate environment. Antisense constructs or sense constructs that are not or cannot be translated are expressly included by this definition. Examples for viral vectors include, but are not limited to retroviral, adenoviral, lentiviral and adeno-associated viral vectors. The viral expression vector provided herein may include nucleic acid sequences encoding for a selectable marker protein (e.g., the human methylguanine methyltransferase mutant P140K/MGMT) to select for cells including the viral expression vector. The viral expression vector may include nucleic acid sequences encoding for an antiviral protein (e.g., C46 fusion inhibitor, Rev M10 protein). The viral expression vector may further include regulatory sequences necessary to express the selectable marker and/or the antiviral protein. The promoter controlling expression of the selectable marker protein and the antiviral protein is referred to herein as “protein promoter.” In embodiments, the viral expression marker includes a protein promoter. In embodiments, the protein promoter is a polymerase II promoter.

As described above the first RNA promoter may be a forward promoter. The recombinant nucleic acid provided herein including embodiments thereof may form part of a viral expression vector. Where the recombinant nucleic acid provided herein including embodiments thereof forms part of a viral expression vector and where the first RNA promoter is a forward promoter, the first RNA promoter has the same transcriptional direction (direction of mRNA synthesis) as the protein promoter or the viral expression vector promoter. Where two promoters have the same transcriptional direction, the polymerase synthesizing (transcribing) mRNA from those promoters uses the same template strand (e.g., sense or antisense). Thus, the antiviral RNA encoding sequences operably linked to (transcriptionally controlled by) the first RNA promoter, are transcribed in the same direction as the genes operably linked to (transcriptionally controlled by) the protein promoter or the viral expression vector promoter, when the first RNA promoter is a forward promoter. The recombinant nucleic acid provided herein including embodiments thereof may further include a second RNA promoter, which promoter may be a reverse promoter. Thus, the second RNA promoter has the opposite transcriptional direction relative to the first RNA promoter. Further, where the second RNA promoter forms part of a viral expression vector, the second RNA promoter may have the opposite transcriptional direction relative to the protein promoter or to the viral expression vector promoter, when the second RNA promoter is a reverse promoter. Thus, in embodiments, the viral entry inhibiting RNA encoding sequence operably linked to (transcriptionally controlled by) the second RNA promoter is transcribed in the opposite direction relative to the genes operable linked to (transcriptionally controlled by) the protein promoter or the antiviral RNA encoding sequences operably linked to the first RNA promoter.

The viral expression vector may further include nucleic acid sequences encoding for viral proteins (e.g., structural proteins, regulatory proteins). Upon expression in a cell these proteins may form a virus-like particle which includes the antiviral polycistronic RNA. Thus, in embodiments, the recombinant nucleic acid forms part of a recombinant viral particle. The antiviral polycistronic RNA or the recombinant nucleic acid encoding the same may be delivered to a cell, tissue or organ using the recombinant viral particle.

The recombinant nucleic acid provided herein may encode a plurality of different species of siRNAs. The antiviral RNA may be a ribozyme, an RNA decoy, an shRNA, a miRNA, or a snoRNA. Thus, in embodiments, the first antiviral RNA encoding sequence encodes a first small interfering RNA (siRNA), the second antiviral RNA encoding sequence encodes a second siRNA and the third antiviral RNA encoding sequence encodes a third siRNA. The first siRNA, second siRNA and third siRNA may independently be a viral transcription inhibiting siRNA (a small RNA inhibiting viral transcription), a viral replication inhibiting siRNA (a small RNA inhibiting viral replication), a viral transcription and replication inhibiting siRNA (a small RNA inhibiting viral transcription and replication), a ribozyme or an RNA decoy. A “ribozyme” as provided herein refers to a ribonucleic acid capable of enzymatically modifying RNA (e.g., cleaving, splicing). An RNA decoy as provided herein is an siRNA, which inhibits the function of a protein (e.g., viral protein or cellular protein) by binding the protein. The RNA decoy may inhibit protein function by preventing the interaction between the protein (e.g., Tat) and its natural interaction partners (e.g., TAR). Further, the binding of an RNA decoy to a protein may alter the subcellular location of the protein thereby inhibiting its activity. In embodiments, the RNA decoy is a U16TAR decoy. In embodiments, the RNA decoy is a U16RBE decoy.

In embodiments, the first siRNA is a viral transcription inhibiting siRNA (a small RNA inhibiting viral transcription), a viral replication inhibiting siRNA (a small RNA inhibiting viral replication), a viral transcription and replication inhibiting siRNA (a small RNA inhibiting viral transcription and replication), a ribozyme or an RNA decoy. In embodiments, the second siRNA is a viral transcription inhibiting siRNA (a small RNA inhibiting viral transcription), a viral replication inhibiting siRNA (a small RNA inhibiting viral replication), a viral transcription and replication inhibiting siRNA (a small RNA inhibiting viral transcription and replication), a ribozyme or an RNA decoy. In embodiments, the third siRNA is a viral transcription inhibiting siRNA (a small RNA inhibiting viral transcription), a viral replication inhibiting siRNA (a small RNA inhibiting viral replication), a viral transcription and replication inhibiting siRNA (a small RNA inhibiting viral transcription and replication), a ribozyme or an RNA decoy. In embodiments, the viral transcription inhibiting siRNA is a Tat siRNA. In embodiments, the viral replication inhibiting siRNA is a Rev siRNA. In embodiments, the viral transcription and replication inhibiting siRNA is a Tat/Rev siRNA. Where the viral transcription and replication inhibiting siRNA is a Tat/Rev siRNA, the siRNA is capable of inhibiting Tat and Rev. In embodiments, the ribozyme is a small nucleolar (sno) RNA. In embodiments, the snoRNA is a U5 ribozyme (e.g., U16U5RZ). In embodiments, the RNA decoy is a snoRNA. In embodiments, the snoRNA is a U16TAR decoy. In embodiments, the snoRNA is a rev binding RNA decoy (e.g., U16RBE) or a Tat binding RNA decoy (e.g., U16TAR).

The recombinant nucleic acid may further include a transcriptional terminator sequence. A transcriptional terminator sequence as provided herein refers to a nucleic acid sequence capable of abrogating RNA transcription. A transcriptional terminator sequence may disrupt the mRNA-DNA-RNA polymerase ternary complex thereby terminating the transcription process. In embodiments, the recombinant nucleic acid includes a first RNA promoter operably linked to: (i) a first antiviral RNA encoding sequence, (ii) a second antiviral RNA encoding sequence and a (iii) third antiviral RNA encoding sequence, wherein the first RNA promoter is a forward promoter, and a transcriptional terminator sequence. In other embodiments, the recombinant nucleic acid includes a first RNA promoter operably linked to: (i) a first antiviral RNA encoding sequence, (ii) a second antiviral RNA encoding sequence and a (iii) third antiviral RNA encoding sequence, wherein the first RNA promoter is a forward promoter; a second RNA promoter operably linked to a viral entry inhibiting RNA encoding sequence, wherein said second promoter is a reverse promoter, and a transcriptional terminator sequence. In embodiments, the transcriptional terminator sequence is an U1 terminator sequence. In embodiments, the transcriptional terminator sequence is downstream of the viral entry inhibiting RNA encoding sequence.

The recombinant nucleic acid may further include a first nucleic acid linker connecting the first antiviral RNA encoding sequence to the second antiviral RNA encoding sequence and a second nucleic acid linker connecting the second antiviral RNA encoding sequence to the third antiviral RNA encoding sequence. A nucleic acid linker as provided herein is a nucleic acid molecule connecting two nucleic acid sequences through covalent binding. In embodiments, the nucleic acid linker includes at least 10 nucleotides. In embodiments, the nucleic acid linker includes at least 20 nucleotides. In embodiments, the nucleic acid linker includes at least 30 nucleotides. In embodiments, the nucleic acid linker includes at least 40 nucleotides. In embodiments, the nucleic acid linker includes at least 50 nucleotides. In embodiments, the nucleic acid linker includes at least 60 nucleotides. In embodiments, the nucleic acid linker includes at least 70 nucleotides. In embodiments, the nucleic acid linker includes at least 80 nucleotides. In embodiments, the nucleic acid linker includes at least 90 nucleotides. In embodiments, the nucleic acid linker includes at least 100 nucleotides. In embodiments, the first nucleic acid linker or the second nucleic acid linker include an intron sequence. In embodiments, the first nucleic acid linker or the second nucleic acid linker include an exon sequence. In embodiments, the first nucleic acid linker or the second nucleic acid linker include an intron sequence or an exon sequence. In embodiments, the first nucleic acid linker or the second nucleic acid linker include an intron sequence and an exon sequence. In embodiments, the first nucleic acid linker and the second nucleic acid linker include an intron sequence and an exon sequence. In embodiments, the intron sequence is a MCM7 intron sequence. In embodiments, the exon sequence is a MCM7 exon sequence.

The recombinant nucleic acid provided herein including embodiments thereof may include an antiviral protein encoding sequence. An antiviral protein encoding sequence refers to a nucleic acid sequence encoding a polypeptide capable of inhibiting viral activity (e.g., replication, transcription, translation, infection). Thus, in embodiments the antiviral protein is an inhibitor of viral replication. In embodiments, the antiviral protein is an inhibitor of viral transcription. In embodiments, the antiviral protein is an inhibitor of viral entry. In embodiments, the antiviral protein is an inhibitor of viral transport. In embodiments, the antiviral protein is an inhibitor of viral packaging. In embodiments, the antiviral protein encoding sequence encodes a C46 fusion inhibitor. In embodiments, the antiviral protein encoding sequence encodes a mutant Rev protein. In embodiments, the mutant Rev protein is a Rev M10 protein. In embodiments, the antiviral protein encoding sequence is downstream of the viral entry inhibiting RNA encoding sequence. In embodiments, the recombinant nucleic acid includes a transcriptional terminator sequence. In embodiments, the transcriptional terminator sequence is an U1 terminator sequence. In embodiments, the transcriptional terminator sequence is downstream of the antiviral protein encoding sequence.

The recombinant nucleic acid provided herein including embodiments thereof may include a fourth antiviral RNA encoding sequence. The fourth antiviral RNA encoding sequence encodes a fourth siRNA. In embodiments, the fourth siRNA is a viral transcription inhibiting siRNA (a small RNA inhibiting viral transcription), a viral replication inhibiting siRNA (a small RNA inhibiting viral replication), a viral transcription and replication inhibiting siRNA (a small RNA inhibiting viral transcription and replication), a ribozyme or an RNA decoy. In embodiments, the viral transcription inhibiting siRNA is a Tat siRNA. In embodiments, the viral replication inhibiting siRNA is a Rev siRNA. In embodiments, the viral transcription and replication inhibiting siRNA is a Tat/Rev siRNA. Where the viral transcription and replication inhibiting siRNA is a Tat/Rev siRNA, the siRNA is capable of inhibiting Tat and Rev. In embodiments, the ribozyme is a small nucleolar (sno) RNA. In embodiments, the snoRNA is a U5 ribozyme (e.g., U16U5RZ). In embodiments, the RNA decoy is a snoRNA. In embodiments, the snoRNA is a U16TAR decoy. In embodiments, the snoRNA is a rev binding RNA decoy (e.g., U16RBE) or a Tat binding RNA decoy (e.g., U16TAR). In embodiments, the RNA decoy is a U16TAR decoy. In embodiments, the RNA decoy is a U16RBE decoy. In embodiments, the fourth siRNA is an RNA decoy. In embodiments, the fourth siRNA is a Tat binding RNA decoy (e.g., U16TAR). In embodiments, the fourth siRNA is a U16TAR decoy. In embodiments, the fourth antiviral RNA encoding sequence is operably linked to a third RNA promoter. In embodiments, the third RNA promoter is a polymerase III promoter. In embodiments, the RNA polymerase III promoter is a small nuclear RNA (snRNA) promoter. In embodiments, the snRNA promoter is a U6 promoter. In embodiments, the third RNA promoter is a forward promoter. In embodiments, the third RNA promoter is located upstream of the protein promoter.

The compositions provided herein including embodiments thereof may include different combinations of antiviral RNA encoding sequences, viral entry inhibiting RNA encoding sequences and antiviral protein encoding sequences. Thus, in embodiments, the recombinant nucleic acid composition includes a first RNA promoter operably linked to: (i) a first antiviral RNA encoding sequence, (ii) a second antiviral RNA encoding sequence and a (iii) third antiviral RNA encoding sequence, wherein the first RNA promoter is a forward promoter and a second RNA promoter operably linked to a viral entry inhibiting RNA encoding sequence, wherein said second promoter is a reverse promoter. In embodiments, the first RNA promoter is a U1 promoter, the first antiviral RNA encoding sequence encodes a Tat/Rev siRNA, the second antiviral RNA encoding sequence encodes a Rev siRNA, the third antiviral RNA encoding sequence encodes a Tat siRNA, the second RNA promoter is a U6 promoter, and the viral entry inhibiting RNA encoding sequence encodes a CCR5 shRNA. In embodiments, the first RNA promoter is a U1 promoter, the first antiviral RNA encoding sequence encodes a Tat/Rev siRNA, the second antiviral RNA encoding sequence encodes a Rev siRNA, the third antiviral RNA encoding sequence encodes a Tat binding RNA decoy (e.g., U16TAR), the second RNA promoter is a U6 promoter and the viral entry inhibiting RNA encoding sequence encodes a CCR5 shRNA. In embodiments, the first RNA promoter is a U1 promoter, the first antiviral RNA encoding sequence encodes a Tat/Rev siRNA, the second antiviral RNA encoding sequence encodes a U5 ribozyme (e.g., U16U5RZ), the third antiviral RNA encoding sequence encodes a Tat binding RNA decoy (e.g., U16TAR), the second RNA promoter is a U6 promoter, and the viral entry inhibiting RNA encoding sequence encodes a CCR5 shRNA. In embodiments, the first RNA promoter is a U1 promoter, the first antiviral RNA encoding sequence encodes a Tat/Rev siRNA, the second antiviral RNA encoding sequence encodes a U5 ribozyme (e.g., U16U5RZ), the third antiviral RNA encoding sequence encodes a Tat binding RNA decoy (e.g., U16TAR), the second RNA promoter is a U6 promoter, the viral entry inhibiting RNA encoding sequence encodes a CCR5 shRNA, the third RNA promoter is a U6 promoter and the fourth antiviral RNA encoding sequence encodes a Tat binding RNA decoy (e.g., U16TAR).

In another aspect, a recombinant nucleic acid encoding an antiviral polycistronic RNA is provided. The recombinant nucleic acid includes a first RNA promoter operably linked to: (i) a first antiviral RNA encoding sequence, a second antiviral RNA encoding sequence and a third antiviral RNA encoding sequence; and (ii) a second RNA promoter operably linked to a viral entry inhibiting RNA encoding sequence. In embodiments, the first RNA promoter is a forward promoter. In embodiments, the first RNA promoter is a reverse promoter. In embodiments, the second RNA promoter is a forward promoter. In embodiments, the second RNA promoter is a reverse promoter. In embodiments, the first RNA promoter is a forward promoter and the second RNA promoter is a reverse promoter.

As described above, the recombinant nucleic acid may form part of a viral expression vector. The viral expression vector provided herein may include nucleic acid sequences encoding for a selectable marker protein (e.g., the human methylguanine methyltransferase mutant P140K/MGMT) to select for cells including the viral expression vector. The viral expression vector may include nucleic acid sequences encoding for an antiviral protein (e.g., C46 fusion inhibitor, Rev M10 protein). The viral expression vector may further include regulatory sequences necessary to express the selectable marker and/or the antiviral protein. In embodiments, the recombinant nucleic acid forms part of a recombinant viral particle.

In embodiments, the first RNA promoter is a RNA polymerase II promoter. In embodiments, the RNA polymerase II promoter is a small nuclear RNA (snRNA) promoter. In embodiments, the snRNA promoter is a U1 promoter.

In embodiments, the first antiviral RNA encoding sequence encodes a first small interfering RNA (siRNA), the second antiviral RNA encoding sequence encodes a second siRNA and the third antiviral RNA encoding sequence encodes a third siRNA. In embodiments, the first siRNA, second siRNA and third siRNA are independently a viral transcription inhibiting siRNA, a viral replication inhibiting siRNA, a viral transcription and replication inhibiting siRNA, a ribozyme or an RNA decoy. In embodiments, the first siRNA is a viral transcription inhibiting siRNA (a small RNA inhibiting viral transcription), a viral replication inhibiting siRNA (a small RNA inhibiting viral replication), a viral transcription and replication inhibiting siRNA (a small RNA inhibiting viral transcription and replication), a ribozyme or an RNA decoy. In embodiments, the second siRNA is a viral transcription inhibiting siRNA (a small RNA inhibiting viral transcription), a viral replication inhibiting siRNA (a small RNA inhibiting viral replication), a viral transcription and replication inhibiting siRNA (a small RNA inhibiting viral transcription and replication), a ribozyme or an RNA decoy. In embodiments, the third siRNA is a viral transcription inhibiting siRNA (a small RNA inhibiting viral transcription), a viral replication inhibiting siRNA (a small RNA inhibiting viral replication), a viral transcription and replication inhibiting siRNA (a small RNA inhibiting viral transcription and replication), a ribozyme or an RNA decoy. In embodiments, the viral transcription inhibiting siRNA is a Tat siRNA. In embodiments, the viral replication inhibiting siRNA is a Rev siRNA. In embodiments, the viral transcription and replication inhibiting siRNA is a Tat/Rev siRNA. Where the viral transcription and replication inhibiting siRNA is a Tat/Rev siRNA, the siRNA is capable of inhibiting Tat and Rev. In embodiments, the ribozyme is a small nucleolar (sno) RNA. In embodiments, the snoRNA is a U5 ribozyme (e.g., U16U5RZ). In embodiments, the RNA decoy is a snoRNA. In embodiments, the snoRNA is a U16TAR decoy. In embodiments, the snoRNA is a rev binding RNA decoy (e.g., U16RBE) or a Tat binding RNA decoy (e.g., U16TAR).

In embodiments, the second RNA promoter is downstream of the third antiviral RNA encoding sequence. In embodiments, the second RNA promoter is a polymerase III promoter. In embodiments, the RNA polymerase III promoter is a small nuclear RNA (snRNA) promoter. In embodiments, the snRNA promoter is a U6 promoter.

In embodiments, the viral entry inhibiting RNA encoding sequence encodes a cellular receptor siRNA. In embodiments, the cellular receptor siRNA is a T cell receptor siRNA. In embodiments, the T cell receptor siRNA is a small hairpin (sh) RNA. In embodiments, the shRNA is a CCR5 shRNA. In embodiments, the shRNA is a CXCR4 shRNA. In embodiments, the viral entry inhibiting RNA encoding sequence encodes a nuclear receptor siRNA. In embodiments, the nuclear receptor siRNA is a transportin 3 (TNPO3) siRNA.

The recombinant nucleic acid provided herein including embodiments thereof may further include a transcriptional terminator sequence. In embodiments, the transcriptional terminator sequence is an U1 terminator sequence. In embodiments, the transcriptional terminator sequence is downstream of the viral entry inhibiting RNA encoding sequence. In embodiments, the recombinant nucleic acid includes a first RNA promoter operably linked to: (i) a first antiviral RNA encoding sequence, (ii) a second antiviral RNA encoding sequence and a (iii) third antiviral RNA encoding sequence, a second RNA promoter operably linked to a viral entry inhibiting RNA encoding sequence and a transcriptional terminator sequence. In embodiments, the transcriptional terminator sequence is an U1 terminator sequence. In embodiments, the transcriptional terminator sequence is downstream of the viral entry inhibiting RNA encoding sequence.

The recombinant nucleic acid provided herein including embodiments thereof may further include a first nucleic acid linker connecting the first antiviral RNA encoding sequence to the second antiviral RNA encoding sequence and a second nucleic acid linker connecting the second antiviral RNA encoding sequence to the third antiviral RNA encoding sequence. In embodiments, the first nucleic acid linker or the second nucleic acid linker include an exon sequence. In embodiments, the first nucleic acid linker or the second nucleic acid linker include an intron sequence or an exon sequence. In embodiments, the first nucleic acid linker or the second nucleic acid linker include an intron sequence and an exon sequence. In embodiments, the first nucleic acid linker and the second nucleic acid linker include an intron sequence and an exon sequence. In embodiments, the intron sequence is a MCM7 intron sequence. In embodiments, the exon sequence is a MCM7 exon sequence.

The compositions provided herein including embodiments thereof may include different combinations of antiviral RNA encoding sequences, viral entry inhibiting RNA encoding sequences and antiviral protein encoding sequences. Thus, in some embodiments, the recombinant nucleic acid composition includes a first RNA promoter operably linked to: (i) a first antiviral RNA encoding sequence, a second antiviral RNA encoding sequence and a third antiviral RNA encoding sequence; and (ii) a second RNA promoter operably linked to a viral entry inhibiting RNA encoding sequence. In embodiments, the first RNA promoter is a U1 promoter, the first antiviral RNA encoding sequence encodes a Tat/Rev siRNA, the second antiviral RNA encoding sequence encodes a Rev siRNA, the third antiviral RNA encoding sequence encodes a Tat siRNA, the second RNA promoter is a U6 promoter and the viral entry inhibiting RNA encoding sequence encodes a CCR5 shRNA. In embodiments, the first RNA promoter is a U1 promoter, the first antiviral RNA encoding sequence encodes a Tat/Rev siRNA, the second antiviral RNA encoding sequence encodes a Rev siRNA, the third antiviral RNA encoding sequence encodes a Tat binding RNA decoy, the second RNA promoter is a U6 promoter, and the viral entry inhibiting RNA encoding sequence encodes a CCR5 shRNA. In embodiments, the first RNA promoter is a U1 promoter, the first antiviral RNA encoding sequence encodes a Tat/Rev siRNA, the second antiviral RNA encoding sequence encodes a U5 ribozyme, the third antiviral RNA encoding sequence encodes a Tat binding RNA decoy, the second RNA promoter is a U6 promoter and the viral entry inhibiting RNA encoding sequence encodes a CCR5 shRNA.

In embodiments, the recombinant nucleic acid includes a fourth antiviral RNA encoding sequence. The fourth antiviral RNA encoding sequence encodes a fourth siRNA. In embodiments, the fourth siRNA is a viral transcription inhibiting siRNA (a small RNA inhibiting viral transcription), a viral replication inhibiting siRNA (a small RNA inhibiting viral replication), a viral transcription and replication inhibiting siRNA (a small RNA inhibiting viral transcription and replication), a ribozyme or an RNA decoy. In embodiments, the viral transcription inhibiting siRNA is a Tat siRNA. In embodiments, the viral replication inhibiting siRNA is a Rev siRNA. In embodiments, the viral transcription and replication inhibiting siRNA is a Tat/Rev siRNA. Where the viral transcription and replication inhibiting siRNA is a Tat/Rev siRNA, the siRNA is capable of inhibiting Tat and Rev. In embodiments, the ribozyme is a small nucleolar (sno) RNA. In embodiments, the snoRNA is a U5 ribozyme (e.g., U16U5RZ). In embodiments, the RNA decoy is a snoRNA. In embodiments, the snoRNA is a U16TAR decoy. In embodiments, the snoRNA is a rev binding RNA decoy (e.g., U16RBE) or a Tat binding RNA decoy (e.g., U16TAR). In embodiments, the RNA decoy is a U16TAR decoy. In embodiments, the RNA decoy is a U16RBE decoy. In embodiments, the fourth siRNA is a RNA decoy. In embodiments, the fourth siRNA is a Tat binding RNA decoy (e.g., U16TAR). In embodiments, the fourth siRNA is a U16TAR decoy. In embodiments, the fourth antiviral RNA encoding sequence is operably linked to a third RNA promoter. In embodiments, the third RNA promoter is a polymerase III promoter. In embodiments, the RNA polymerase III promoter is a small nuclear RNA (snRNA) promoter. In embodiments, the snRNA promoter is a U6 promoter. In embodiments, the third RNA promoter is a forward promoter. In embodiments, the third RNA promoter is located upstream of the protein promoter.

### Cellular Compositions

The recombinant nucleic acid compositions provided herein including embodiments thereof may be expressed by a cell (e.g., mammalian cell), tissue or organ. Upon expression in a cell siRNA molecules as described above are formed, and said siRNA molecules confer antiviral activity to the cell. Thus, in another aspect, a mammalian cell including a recombinant antiviral polycistronic RNA is provided. The recombinant antiviral polycistronic RNA includes (i) a first antiviral RNA, a second antiviral RNA and a third antiviral RNA; and (ii) a viral entry inhibiting RNA. In embodiments, the first antiviral RNA, the second antiviral RNA and the third antiviral RNA is a small interfering RNA (siRNA). In embodiments, the siRNA is a viral transcription inhibiting siRNA, a viral replication inhibiting siRNA, a viral transcription and replication inhibiting siRNA, a ribozyme or an RNA decoy. In embodiments, the viral transcription inhibiting siRNA is a Tat siRNA. In embodiments, the viral replication inhibiting siRNA is a Rev siRNA. In embodiments, the viral transcription and replication inhibiting siRNA is a Tat/Rev siRNA. In embodiments, the ribozyme is a snoRNA. In embodiments, the ribozyme is a U5 ribozyme (e.g., U16U5RZ). In embodiments, the RNA decoy is a snoRNA. In embodiments, the RNA decoy is a rev binding RNA decoy or a Tat binding RNA decoy.

In embodiments, the viral entry inhibiting RNA is a cellular receptor siRNA. In embodiments, the cellular receptor siRNA is a T cell receptor siRNA. In embodiments, the T cell receptor siRNA is a small hairpin (sh) RNA. In embodiments, the shRNA is a CCR5 shRNA. In embodiments, the shRNA is a CXCR4 shRNA. In embodiments, the viral entry inhibiting RNA is a nuclear receptor siRNA. In embodiments, the nuclear receptor siRNA is a transportin 3 (TNPO3) siRNA.

In embodiments, the mammalian cell includes an antiviral protein. In embodiments, the antiviral protein is a C46 fusion inhibitor. In embodiments, the antiviral protein is a mutant Rev protein. In embodiments, the mutant Rev protein is a Rev M10 protein. In embodiments, the first antiviral RNA is a Tat/Rev siRNA, the second antiviral RNA is a Rev siRNA, the third antiviral RNA is a Tat siRNA, and the viral entry inhibiting RNA is a CCR5 shRNA. In embodiments, the first antiviral RNA is a Tat/Rev siRNA, the second antiviral RNA is a Rev siRNA, the third antiviral RNA is a Tat binding RNA decoy, and the viral entry inhibiting RNA is a CCR5 shRNA. In embodiments, the first antiviral RNA is a Tat/Rev siRNA, the second antiviral RNA is a U5 ribozyme, the third antiviral RNA is a Tat binding RNA decoy, and the viral entry inhibiting RNA is a CCR5 shRNA.

### Kits

In another aspect, a kit including a recombinant antiviral polycistronic RNA is provided. The recombinant antiviral polycistronic RNA includes a first antiviral RNA, a second antiviral RNA and a third antiviral RNA; and (ii) a viral entry inhibiting RNA. In embodiments, the kit includes instructions for making a cell expressing the recombinant antiviral polycistronic RNA. In embodiments the kit includes a recombinant antiviral polycistronic RNA or a recombinant nucleic acid encoding the recombinant antiviral polycistronic RNA described herein, including in any aspect, embodiment, example, claim, or figure. In embodiments, the kit includes a composition or mixture that includes a first antiviral RNA, a second antiviral RNA and a third antiviral RNA; and a viral entry inhibiting RNA.

In embodiments, the first antiviral RNA, the second antiviral RNA and the third antiviral RNA is a small interfering RNA (siRNA). In embodiments, the siRNA is a viral transcription inhibiting siRNA, a viral replication inhibiting siRNA, a viral transcription and replication inhibiting siRNA, a ribozyme or an RNA decoy. In embodiments, the viral transcription inhibiting siRNA is a Tat siRNA. In embodiments, the viral replication inhibiting siRNA is a Rev siRNA. In embodiments, the viral transcription and replication inhibiting siRNA is a Tat/Rev siRNA. In embodiments, the ribozyme is a snoRNA. In embodiments, the ribozyme is a U5 ribozyme. In embodiments, the RNA decoy is a snoRNA. In embodiments, the RNA decoy is a rev binding RNA decoy or a Tat binding RNA decoy. In embodiments, the viral entry inhibiting RNA is a cellular receptor siRNA. In embodiments, the cellular receptor siRNA is a T cell receptor siRNA. In embodiments, the T cell receptor siRNA is a small hairpin (sh) RNA. In embodiments, the shRNA is a CCR5 shRNA. In embodiments, the shRNA is a CXCR4 shRNA.

In embodiments, the viral entry inhibiting RNA is a nuclear receptor siRNA. In embodiments, the said nuclear receptor siRNA is a transportin 3 (TNPO3) siRNA. In embodiments, the first antiviral RNA is a Tat/Rev siRNA, the second antiviral RNA is a Rev siRNA, the third antiviral RNA is a Tat siRNA, and the viral entry inhibiting RNA is a CCR5 shRNA. In embodiments, the first antiviral RNA is a Tat/Rev siRNA, the second antiviral RNA is a Rev siRNA, the third antiviral RNA is a Tat binding RNA decoy, and the viral entry inhibiting RNA is a CCR5 shRNA. In embodiments, the first antiviral RNA is a Tat/Rev siRNA, the second antiviral RNA is a U5 ribozyme, the third antiviral RNA is a Tat binding RNA decoy, and the viral entry inhibiting RNA is a CCR5 shRNA.

In another aspect, a kit including a recominant viral particle including the recombinant nucleic acid provided herein including embodiments thereof is provided.

In another aspect, a kit including a recominant viral particle including a recombinant antiviral polycistronic RNA is provided. The recombinant antiviral polycistronic RNA includes a first antiviral RNA, a second antiviral RNA and a third antiviral RNA; and (ii) a viral entry inhibiting RNA.

### Pharmaceutical Compositions and Methods of Treatment

In another aspect, a pharmaceutical composition including a pharmaceutically acceptable excipient and a recombinant viral particle including a recombinant nucleic acid as provided herein including embodiments thereof is provided.

In another aspect, a method of treating an infectious disease in a subject in need thereof is provided. The method includes administering to the subject a therapeutically effective amount of a recombinant viral particle including a recombinant nucleic acid as provided herein including embodiments thereof. In embodiments, the infectious disease is caused by a virus. In embodiments, the virus is HIV. In embodiments, the subject suffers from AIDS.

In another aspect, a method of treating an infectious disease in a subject in need thereof is provided. The method includes administering to the subject a mammalian cell including a recombinant antiviral polycistronic RNA as provided herein including embodiments thereof. In embodiments, the mammalian cell is derived from the patient. In embodiments, the mammalian cell is derived from a healthy subject. In embodiments, the mammalian cell is formed by transfection with a recombinant nucleic acid encoding an antiviral polycistronic RNA as provided herein including embodiments thereof.

In another aspect, a method of inhibiting HIV replication in a patient is provided. The method includes administering to the patient a therapeutically effective amount of a recombinant viral particle including a recombinant nucleic acid as provided herein including embodiments thereof, thereby inhibiting HIV replication in the patient.

In another aspect, a method of inhibiting HIV replication in a patient is provided. The method includes administering to the subject a mammalian cell including a recombinant antiviral polycistronic RNA as provided herein including embodiments thereof, thereby inhibiting HIV replication in the patient. In embodiments, the mammalian cell is derived from the patient. In embodiments, the mammalian cell is derived from a healthy subject. In embodiments, the mammalian cell is formed by transfection with a recombinant nucleic acid encoding an antiviral polycistronic RNA as provided herein including embodiments thereof.

### EXAMPLES

### Example 1

Combinational therapy with small RNA inhibitory agents against multiple viral targets allows efficient inhibition of viral production by controlling gene expression at critical time points. Here Applicants explore combinations of different classes of therapeutic anti-HIV-1 RNAs expressed from within the context of an intronic MCM7 platform that naturally harbors three miRNAs. Applicants replaced the endogenous miRNAs with anti-HIV small RNAs, including siRNAs targeting HIV-1 tat and rev messages that function to induce post-transcriptional gene silencing by the RNA interference pathway, a nucleolar-localizing RNA ribozyme that targets the conserved U5 region of HIV-1 transcripts for degradation, and finally nucleolar TAR and RBE RNA decoys designed to sequester HIV-1 Tat and Rev proteins inside the nucleolus. Applicants demonstrate the versatility of the MCM7 platform in expressing and efficient processing of the siRNAs as miRNA mimics along with nucleolar small RNAs. Furthermore, three of the combinatorial constructs tested potently suppressed viral replication during a one-month HIV challenge, with greater than 5-logs inhibition compared to untransduced, HIV-1 infected CEM T-lymphocytes. One of the most effective constructs contains an anti-HIV siRNA combined with a nucleolar-localizing U5 ribozyme and TAR decoy. This represents the first efficacious example of combining Drosha processed siRNAs with snoRNP processed nucleolar RNA chimeras from a single intron platform for effective inhibition of viral replication. Moreover, Applicants demonstrated an enrichment/selection for cells expressing levels of the anti-viral RNAs which provide optimal inhibition under the selective pressure of HIV. The combinations of si/sno RNAs represent a new paradigm for combinatorial RNA-based gene therapy applications.

Generation of the MCM7-snoRNA Constructs

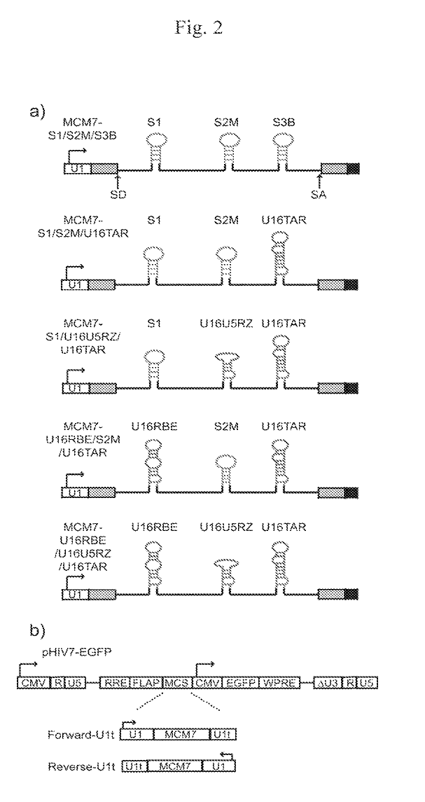

Previously, Applicants engineered and optimized a polycistronic miRNA cluster located in an intron of the protein encoding gene MCM7 as a siRNA multiplexing platform (Aagaard, L. A. et al., Gene Ther., 15, 1536-1549 (2008)). This platform which Applicants refer to as MCM7 was engineered to simultaneously express three anti-HIV siRNAs targeted to the common exon shared by tat/rev (S1), rev (S2M), and tat (S3B), respectively (MCM7-S1/S2M/S3B in FIG. 2a) from a single RNA Pol II human U1 promoter. Moreover, Applicants demonstrated the potential versatility of this platform by co-expressing an U16TAR snoRNA inserted in the position of the S3B subunit. Given the demonstration of co-expression of siRNAs and snoRNAs, Applicants hypothesized that it should be possible to insert the chimeric snoRNAs into any of the three miRNA positions to obtain processing of these small RNAs. If such was the case, Applicants could then examine different combinations of chimeric snoRNAs and siRNAs co-expressed in the same transcript. To test this hypothesis Applicants inserted the U16RBE and U16U5RZ snoRNAs into the MCM7-S1/S2M/U16TAR construct by replacing either the S1 or S2M units. This resulted in the creation of three novel constructs harboring multiple snoRNA chimeras with different targets and mechanisms of action as shown in FIG. 2a.

The original MCM7-S1/S2M/S3B and MCM7-S1/S2M/U16TAR constructs were subcloned into the pHIV7-EGFP lentiviral vector (Yam, P. Y. et al., Mol Ther., 5, 479-484 (2002)) in the reverse orientation with respect to the packaging CMV promoter to prevent splicing of the MCM7 intron during vector packaging (Aagaard, L. A. et al., Gene Ther., 15, 1536-1549 (2008)). Alternatively, the HIV-1 Rev protein used in the packaging process suppresses transcript splicing suggesting Applicants could also orient the U1-MCM7 intron in the same transcriptional direction as the CMV packaging promoter. To test these possibilities, Applicants cloned the MCM7 transgene in both forward and reverse orientations with U1 promoter-specific termination sequence (FIG. 2b) and compared packaging efficiencies. Interestingly the packaging efficiencies were greater than 100-fold better in constructs with the transgene cloned in the forward orientation (Table 1). Applicants therefore used the forward orientations for transduction of CEM T-lymphocytes to produce cell lines that stably expressed the various combinations of anti-HIV RNAs.

Proper Processing and Expression of the Functional Small RNAs in Target Cells

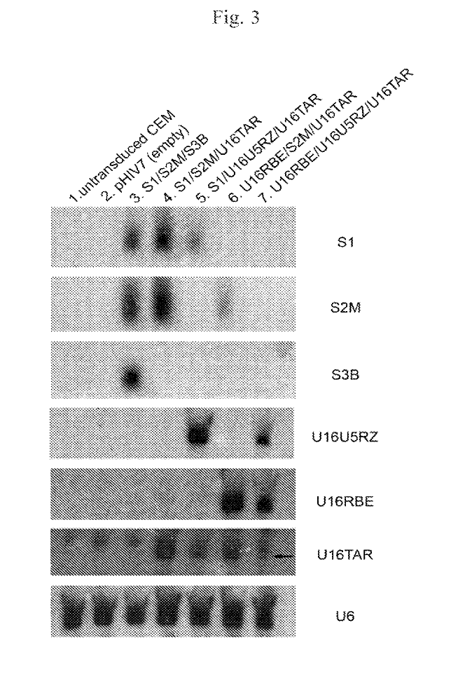

To determine and verify whether the RNA expression of each unit within the combinatorial vector was properly transcribed and processed, a Northern blotting analysis was performed on stably transduced CEM T-lymphocytes (FIG. 3). After electrophoresis of the RNA samples, the blots were hybridized with probes specific for each of the RNAs. RNA expression was detected for each unit in the various constructs, with expected sizes of about 21 nt for siRNAs and about 132 nt for U16 chimeric snoRNAs, indicating efficient processing of the RNAs from the polycistronic transcript. It is interesting to note that certain RNA combinations express lower levels of small RNAs in this platform, implying proper processing of both si- and snoRNA within the same intron is an intricate balance between the Drosha/DGCR8 and the snoRNP pathways and furthermore may be position-dependent (Hirose, T., Shu, M. D., and Steitz, J. A., Mol Cell, 12, 113-123 (2003)).

### Suppression of Viral Replication in CEM T-Lymphocytes Expressing the MCM7 Intron Containing Anti-HIV Small RNAs.

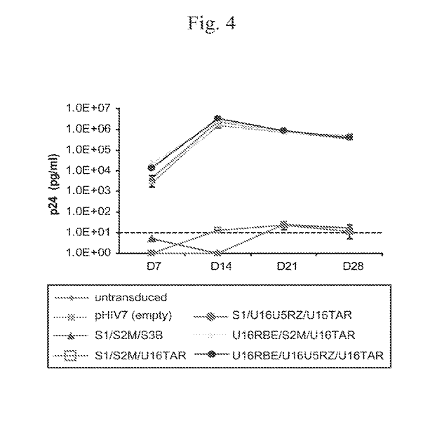

Applicants next addressed the issue of functionality of the small RNAs as measured by antiviral activity. Long term inhibition of viral replication in CEM T-lymphocytes stably expressing the MCM7 constructs was evaluated by viral challenge assays using the NL4-3 strain of HIV-1 at an MOI of 0.01. Virus replication was followed for 28 days by monitoring viral capsid p24 levels in the culture supernatant at the indicated time points (FIG. 4). Three out of the five constructs, MCM7-S1/S2M/S3B, MCM7-S1/S2M/U16TAR, and MCM7-S1/U16U5RZ/U16TAR, showed extremely potent anti-HIV activity, providing greater than a S-log reduction in p24 output, with almost non-detectable p24 during this one-month challenge.

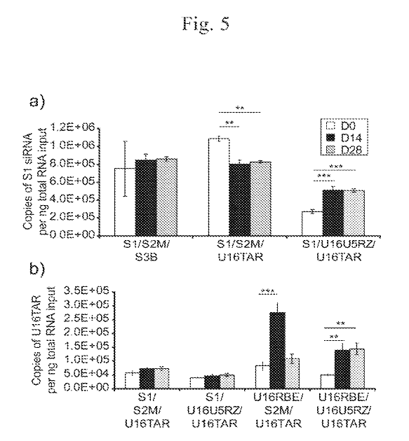

Having achieved strong suppression of viral replication during the challenge assay in CEM T-lymphocytes, Applicants were interested in correlating gene expression to functionality. Applicants hypothesized that HIV-1 would provide selective pressure to enrich for transduced cells with levels of anti-HIV RNA gene expression that effectively suppress replication. If this is the case, Applicants would expect selection for cells with optimal RNA expression levels during the time course of the HIV-1 challenge. Applicants utilized qRT-PCR to measure S1 siRNA and U16TAR decoy RNA expression (FIGS. 5a and 5b, respectively). Cells transduced with the MCM7-S1/U5RZ/TAR construct had a significant 2-fold enrichment for S1 siRNA expression (p<0.001), while interestingly, cells transduced with the MCM7-S1/S2M/TAR construct had a significant 20% reduction for S1 siRNA expression (p<0.01). Significant enrichment for the U16 TAR RNA decoy was observed only in constructs that did not inhibit HIV. These data are consistent with the mechanism of action for each of the small RNAs with siRNA being catalytic and the decoys being stoichiometric in sequestering their targets. Taken together, these data suggest that under the selective pressure of HIV, there is an enrichment/selection for cells expressing levels of the anti-viral RNAs which provide optimal inhibition in the absence of toxicity.

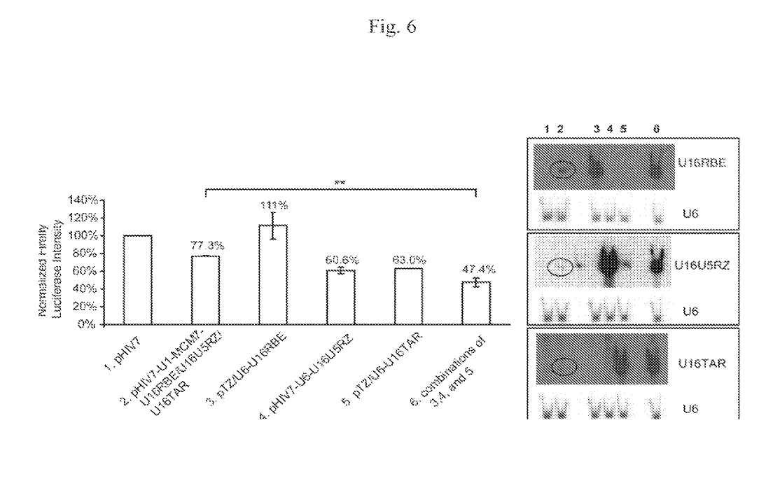

To investigate whether combinations of the various inhibitory RNAs were more efficacious as inhibitors than single antiviral RNAs, Applicants utilized a dual luciferase reporter assay in which Applicants transiently co-transfected a replication-deficient pNL4-3 proviral DNA harboring the firefly luciferase gene in the HIV-1 Nef gene (pNL4-3.Luc.R-.E, catalog #3418 from NIH AIDS reagent and repository, (Connor, R. I. et al., Virology, 206, 935-944 (1995); He, J. et al., J Virol., 69, 6705-6711 (1995)) and the anti-HIV RNAs driven either by the U1 or U6 promoter. The pNL4-3 luciferase construct maintains targets for each of the small RNAs in all the transcripts, both spliced and unspliced and therefore luciferase readouts can be utilized as quantitative readouts of viral inhibition. Applicants observed a general trend correlating knockdown activity with small RNA expression (FIG. 6), consistent with their mechanism of action by increasing target cleavage in the case of U16U5RZ or by sequestering the Tat protein in the case of U16TAR. Applicants were surprised to see that the U16RBE did not exhibit any antiviral activity in this reporter assay, possibly because of overwhelming production of luciferase-labeled viral transcripts during transient transfection and the fact that it is non-catalytic. Overall, these observations suggest that the gene expression level is one of the determinants of an efficient RNA-based therapy.

The use of combinations of small molecule drugs in the highly active anti-retroviral therapy (HAART) to stop or thwart HIV propagation has had a major impact on delaying the progression from HIV-1 infection to the development of AIDS. Despite this progress, there are problems associated with a lifelong use of antiviral drug therapy, including toxicity, the emergence of virus resistant to multiple drugs, and the cost of a daily medication. Gene therapy of human T-lymphocytes and/or hematopoietic progenitor cells can be considered as a potential replacement or supplement to the current anti-HIV-1 therapies. In similarity to small molecule therapies where combinations of drugs targeting different steps in the viral replication cycle have been most effective, Applicants believe that therapeutic RNAs must also be used in combinations to block various stages of the viral replication cycle to mitigate viral escape. Based on recent findings of both HIV-1 viral RNA transcripts and proteins localize in the nucleolus, Applicants have previously demonstrated that nucleolar-localizing small RNAs can be potent therapeutic agents. For example, Applicants had previously succeeded in inhibiting HIV-1 replication by individually expressing snoRNA chimeras, including the U16TAR and U16RBE RNA decoys that sequester the HIV-1 Tat and Rev proteins in the nucleolus, respectively (Michienzi et al., 2006; Michienzi et al., 2002). In addition, Applicants also demonstrated that a nucleolar-localizing ribozyme targeting a conserved U5 sequence present in all HIV-1 transcripts had excellent HIV-1 inhibitory function (Michienzi, A. et al., Proc Natl Acad Sci USA, 99, 14047-14052 (2002); Michienzi, A. et al., AIDS Res Ther., 3, 13 (2006; Unwalla, H. J. et al., Mol Ther., 16, 1113-1119 (2008)). As a combinatorial approach to incorporate anti-HIV small RNAs with different mechanisms of action and target specificity, Applicants multiplexed the aforementioned snoRNA chimeras in addition to siRNAs that cleave tat and rev mRNAs with the goal to block all transcript production and efficiently achieve suppression of HIV-1 replication.

Applicants chose to use a single promoter and an intron-based platform to express combinations of siRNAs and snoRNAs. Both miRNAs and snoRNAs are processed from introns, thereby providing a rationale for Applicants' approach (Hirose, T., Shu, M. D., and Steitz, J. A., Mol Cell, 12, 113-123 (2003)). Applicants previously engineered and optimized the polycistronic miRNA cluster referred to as MCM7 to co-expresses three anti-HIV siRNAs from a single Pol II human U1 promoter (Aagaard, L. A. et al., Gene Ther., 15, 1536-1549 (2008)). Applicants have now carefully examined several aspects of using this system in a lentiviral vector backbone platform. The current constructs were further optimized for packaging efficiency by cloning the MCM7 transgene in the forward direction with respect to the CMV promoter in the lentiviral pHIV7-EGFP vector with the U1-specific transcriptional termination sequence. Applicants were surprised to find the superior packaging efficiency of Applicants' constructs, especially those with the transgene in the forward orientation, compared to the empty pHIV7-EGFP vector. The expression of the anti-HIV RNAs might be expected to negatively impact the transcription of the full-length viral RNA genome during packaging since Applicants' pHIV7-EGFP lentiviral vector is dependent on HIV-1 Rev for packaging. Since all of constructs contain at least one small RNA against HIV-1 Rev, it was expected the viral titer of the constructs might be lower or equivalent at best to the parental pHIV7-EGFP vector. Applicants have previously overcome this challenge by increasing the amount of HIV Rev expressing plasmid (Li, M., and Rossi, J. J., Methods Mol Biol., 309, 261-272 (2005a)) or by inclusion of a plasmid that expresses an Ago2-targeting shRNA (Harris Soifer, unpublished) during packaging to minimize the RNAi activity in cells during packaging. In the present case, Applicants did not find an advantage to down-regulating Ago2 (data not shown) since the siRNA expression levels are relatively low compared to Pol III transcribed shRNAs and during packaging these are not effectively down-regulating the viral transcripts. Applicants also postulate that the insertion of the MCM7 cassette produces a larger viral transcript (5.4 kb) whose size is closer to the natural HIV-1 RNA genome (9 kb) and therefore more favorable for packaging compared to the parental empty vector (3.9 kb). Indeed Applicants found a 2.5-fold increase in viral titer when the parental MCM7 intron lacking anti-HIV RNAs was incorporated (data not shown) in similarity to Applicants' gene-therapy constructs carrying anti-HIV RNAs. Second, Applicants observed the effect of transgene directionality on packaging efficiency, with the forward orientation yielding greater than 100-fold higher production of virus. It is likely the transgene RNA transcript, when expressed from the U1 promoter in the reverse orientation could create an opposing transcript during packaging which negatively impacts on levels of expression from antisense effect.

In this study Applicants have demonstrated the versatility of the MCM7 platform for expressing multiple siRNAs as miRNA mimics as well as snoRNAs from the polycistronic transcript, and efficient processing into mature and functional small RNAs that are readily detectable through the Northern blotting analysis. Long term inhibition of viral replication was evaluated by challenging stably transduced CEM T-lymphocytes with HIV-1 NL4-3. The results of these analyses demonstrated that the MCM7-S1/S2M/S3B, MCM7-S1/S2M/U16TAR, and MCM7-S1/U16U5RZ/U16TAR constructs conferred complete protection against viral replication and spread during the one-month challenge. Interestingly, the MCM7-U16RBE/S2M/U16TAR and MCM7-U16RBE/U16U5RZ/U16TAR constructs did not significantly inhibit HIV replication despite the fact that the small RNAs were actively expressed and readily detectable by Northern blotting. The U16 chimeras in these constructs had demonstrated antiviral activity when individually expressed from the parental vector with the Pol III U6 promoter (Michienzi, A. et al., AIDS Res Ther., 3, 13 (2006; Michienzi, A. et al., Proc Natl Acad Sci USA, 99, 14047-14052 (2002); Unwalla, H. J. et al., Mol Ther., 16, 1113-1119 (2008)). This discrepancy is most likely related to the difference in expression levels of these RNAs in the context of the intronic MCM7 platform driven by the Pol II U1 promoter versus independently from the Pol III U6 promoter.

The importance of optimal levels of RNA expression for anti-HIV activity and cell viability is supported by the observation that there was selection for transduced CEM T-lymphocytes with optimal anti-HIV RNA expression during HIV infection. This phenomenon has also been observed for transduced CEM T-lymphocytes harboring a single copy of the transgene (data not shown), reflecting selection of cells with more transcriptionally active integration sites. Applicants evaluated the overall RNA expression with qRT-PCR and showed persistent expression during challenge and therefore it is likely that there is selective pressure for cells with optimal expression to provide antiviral activity in the absence of cellular toxicity.

In addition to RNA expression levels as a determinant for the effectiveness of a RNA-based gene therapy, the nature of the small RNAs should also be considered and carefully balanced between toxicity and therapeutic efficacy. Because RNA decoys act as “sponges” and therefore function in a stoichiometric fashion, the expression level needs to be sufficiently high to achieve therapeutic efficacy, whereas siRNAs and ribozymes are capable of multiple turnover by cleaving their targets in a catalytic manner and should be functional with lower copies per cell. Constructs with the most potent antiviral activities in the context of the MCM7 intron platform tended to have higher RNA expression levels and usually contained more than two RNA agents that are catalytic in nature, such as a siRNA or ribozyme. It is interesting to note that all the constructs that exhibit antiviral activity in the viral challenge assay contain the S1 siRNA that targets both the HIV-1 tat and rev messages. Although Applicants' current data cannot demonstrate whether the other two small RNAs in the constructs have additive effects in antiviral activity, the principle of the combinational therapy is to reduce viral escape in a long term setting. Applicants have previously demonstrated the combination of three is superior than two and better than single small RNA agents in prolonging anti-HIV protection in long term setting in a viral challenge assay (Li, M., and Rossi, J. J., Methods Mol Biol., 309, 261-272 (2005a)).

In summary, these studies represent the first example of incorporating combinations of snoRNA-based agents with siRNA-based agents within a single expression platform driven by a single Pol II promoter. Applicants demonstrated the versatility of the MCM7 platform for expressing a variety of small anti-viral RNAs in addition to miRNAs. Applicants also demonstrated superior packaging of these constructs versus the parental empty pHIV7-EGFP lentiviral vector. The enhanced packaging efficiency was especially pronounced when the transgene was cloned in the forward orientation with respect to the packaging CMV promoter. Finally, after HIV-1 challenges of CEM T-lymphocytes transduced with the various RNA combinations, Applicants found three small RNA combinations, MCM7-S1/S2M/S3B, MCM7-S1/S2M/U16TAR, and MCM7-S1/U16U5RZ/U16TAR that strongly inhibited viral replication during the one-month challenge. Applicants also found that the pressure of HIV-1 infection resulted in selection of cells with optimal levels anti-HIV gene expression. The two RNA combinations that contained two or more nucleolar RNAs did not significantly inhibit HIV replication, perhaps owing to the non-catalytic nature of RNA decoys versus the siRNAs and ribozyme. Applicants' results suggest these factors should be carefully considered in designing an efficient RNA-based gene therapy.

Protein Transgene

Various protein transgenes can be expressed from Pol II protein promoter. Protein transgene can be antiviral, such as C46 fusion inhibitor or Rev M10 protein or selectable markers to enrich for gene modified cells, such as the P140K mutant of human methylguanine methyltransferase (P140K MGMT). Protein promoters are typically Pol II, such as human EF1 alpha, CMV, human Ubiquitin, SFFV. Applicants utilized a self-cleaving P2A peptide to express multiple protein transgenes (EGFP and P140K MGMT) from Applicants' vectors.

MCM7 Platform

Endogenous polycistronic miRNA cluster (miR-106b-miR-93-miR-25) in the intron of the protein encoding MCM7 gene is engineered as a multiplexing platform to co-express three small RNAs (RNA1, RNA2, RNA3). There are many types of small RNAs that can be expressed from this platform, including small interfering RNAs (siRNAs) and small nucleolar RNAs (snoRNAs). siRNAs are expressed as primary microRNA (pri-miRNA) that requires endogenous RNA interference machinery for processing then gene silencing. SiRNAs targeting any gene of interest can be incorporated, including endogenous or viral genes. Furthermore, dual-targeting siRNA (i.e. bifunctional siRNAs) that target two separate genes or identical gene at two different locations can also be utilized. Examples of endogenous genes important for HIV viral replication include but are not limited to CCR5, CXCR4, and TNPO3. Examples of viral targets include but are not limited to HIV Tat, HIV Rev, and a common exon region shared between Tat and Rev mRNA. Applicants utilized siRNAs targeting HIV Tat (S3B), HIV Rev (S2M), and a common shared exon of Tat and Rev (S1) in Applicants' vector (S1/S2M/S3B).

SnoRNAs can also be successfully incorporated and expressed from the MCM7 platform. These are nucleolar localizing anti-HIV small RNAs constructed with the endogenous U16 snoRNA as a scaffold with the apical loop substituted for various anti-HIV elements. The conserved box C/D domain in U16 snoRNA is sufficient for nucleolar properties.

U16U5RZ is a nucleolar localizing RNA hammerhead ribozyme that recognizes the target by standard Watson-Crick base pairing and cleaves a conserved U5 region in the HIV UTR. U16RBE and U16TAR are nucleolar RNA decoys that sequester HIV Rev and Tat proteins, respectively, into the nucleolus. In U16RBE, the minimal domain within Rev Response element required for interaction with HIV Rev, the Rev binding element (RBE), is substituted into the apical loop of U16 snoRNA. On the other hand, in U16TAR, the minimal transactivation response element hairpin structure is inserted. There were 5 si-/snoRNA combinations that Applicants constructed and tested (See Table 2). In all cases, small RNA transgene are expressed and processed correctly as demonstrated by the northern blotting analysis (FIG. 3).

Applicants also evaluated antiviral activity of these triple constructs with an in vitro viral challenge assay. One million untransduced and stable CEM T lymphocytes were challenged in triplicate with the NL4-3 strain of HIV-1 at an MOI of 0.01, and culture supernatants were collected weekly for the HIV-1 p24 antigen ELISA to evaluate viral replication. Three constructs, MCM7-S1/S2M/S3B, MCM7-S1/S2M/U16TAR, and MCM7-S1/U16U5RZ/U16TAR, showed potent antiviral activity with almost no detectable viral load during the 1-month challenge assay. Therefore, Applicants continued Applicants' construct development effort with only these three RNA combinations (FIG. 4). In FIG. 4 the dashed line represents the low detection limit of the p24 assay. Three constructs, MCM7-S1/S2M/S3B, MCM7-S1/S2M/U16TAR, and MCM7-S1/U16U5RZ/U16TAR, showed potent antiviral activity with almost no detectable viral load during the 1-month challenge assay.

RNA Promoter 1/Termination Seq 1/MCM7 Transgene Orientation

The MCM7 cassette is driven by single Pol II U1 promoter and terminated by an U1-specific termination sequence. This configuration allows the MCM7 platform to be expressed in the forward orientation in the pHIV7 lentiviral backbone. Other Pol II promoters can also be utilized, such as human EF1 alpha, CMV, human Ubiquitin, SFFV, and tissue-specific promoters to engineer tissue specific RNA transgene expression, with the termination sequence substituted with SV40 or BGHpA termination sequence and the requirement that MCM7 platform be expressed in the reverse orientation in the pHIV7 lentiviral backbone.

Applicants have tested the following combinations of promoter/termination sequences and transgene orientations in the lentiviral vector (In each configuration, all 5 RNA combinations listed in 2 were construct giving a combination of 15 candidates). See Table 3 and Table 4. Applicants found the combination U1 promoter and U1 termination sequence with MCM7 cassette in forward orientation in the pHIV7 lentiviral vector gave the best packaging efficiency with similar RNA expression and continued Applicants' vector construct development effort with this configuration.

RNA Promoter 2/RNA4/Termination Seq 2/Transgene Orientation

To increase the functionality of the MCM7 platform, Applicants wish to incorporate additional antiviral small RNAs to enhance the efficacy of Applicants' vectors. The additional small RNA transgene is independently expressed from a Pol III promoter and therefore terminated by the Pol III termination sequence (a consecutive series of 5 to 6 uracil nucleotides). Examples of Pol III promoters include but not limited to U6, H1, tRNA^{Lys}, tRNA^{Ser}, tRNA^{Arg} and examples of small RNA transgene include but not limited to RNA decoys, RNA ribozymes, and siRNAs expressed either as a short hairpin RNA (shRNA) or as a precursor miRNA (pre-miRNA). SiRNAs targeting any gene of interest can be incorporated, including endogenous or viral genes. Furthermore, dual-targeting siRNA (i.e. bifunctional siRNAs) that target two separate genes or identical gene at two different locations can also be utilized. See Table 5. Among the various tRNASer-CCR5 constructs, Applicants optimized the cassette orientation for transgene expression, investigated the efficiency of CCR5 and HIV target knockdown and antiviral potency, and evaluated lentiviral packaging efficiency.

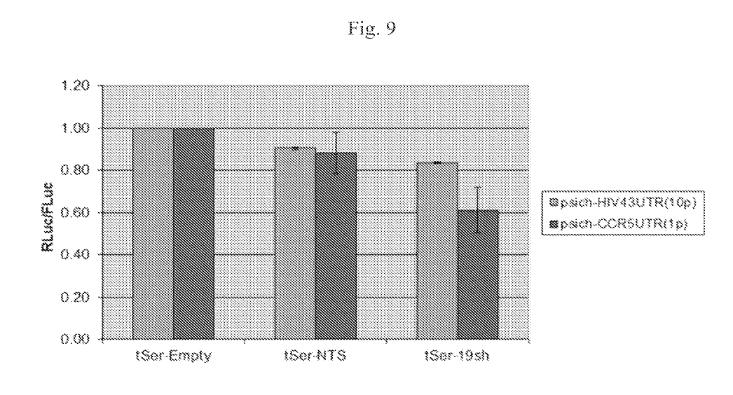

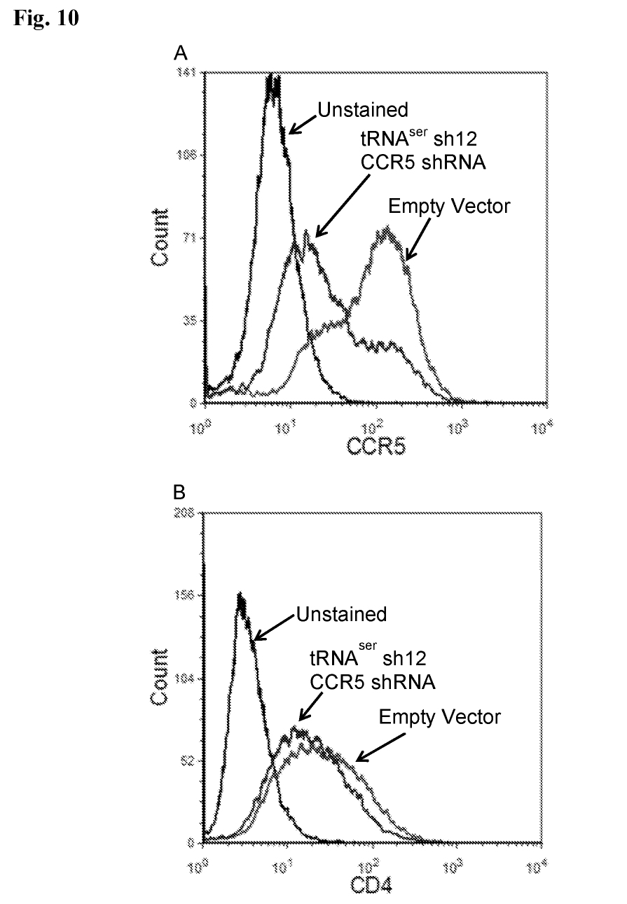

To further verify the biological activity of the mature siRNA sequences produced from the tRNASer-shRNA cassette, Applicants performed a psi-check assay to monitor down-regulation of CCR5 and HIV UTR targets. In this experiment the target sequence is cloned in the 3′ UTR of the reporter Renilla luciferase gene and the fusion transcript is subject to gene silencing by RNA interference. The firefly luciferase reporter serves as a mean to normalize for differences in transfection efficiency. The ratio of Renilla and firefly luciferase expression provides a measure of gene silencing. In this context, bifunctionality siRNAs expressed as a pre-miRNA or as a shRNA are both capable of mediating HIV and CCR5 target knockdown (FIG. 9). Knowing Applicants' mature siRNA sequences can down-regulate target mRNA, Applicants investigated whether this can directly translate to a decrease in CCR5 surface expression. Applicants utilized a cell line that over-expresses CCR5 and transiently transfected Applicants' constructs to monitor CCR5 expression by flow cytometry. Specific decrease in CCR5 expression was only observed with cells transfected with tRNA^{Ser}-CCR5-12sh cassette (FIG. 10).

Collectively based on these data, Applicants incorporated tRNA^{Ser}-CCR5-12sh cassette into the MCM7 platform. Lentiviral packaging efficiency was similar in the presence and absence of this additional cassette and independent of the orientation of the cassette. Because the reverse orientation of this cassette consistently gives higher transgene expression, Applicants determined this is the most optimal configuration for further development. See Table 6.

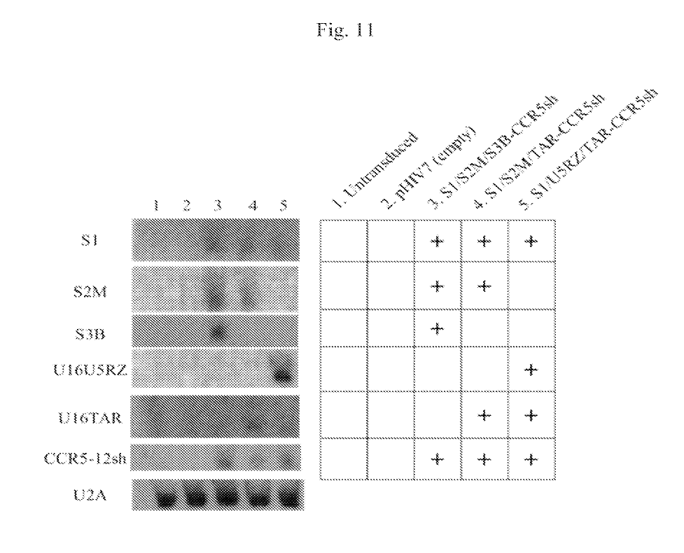

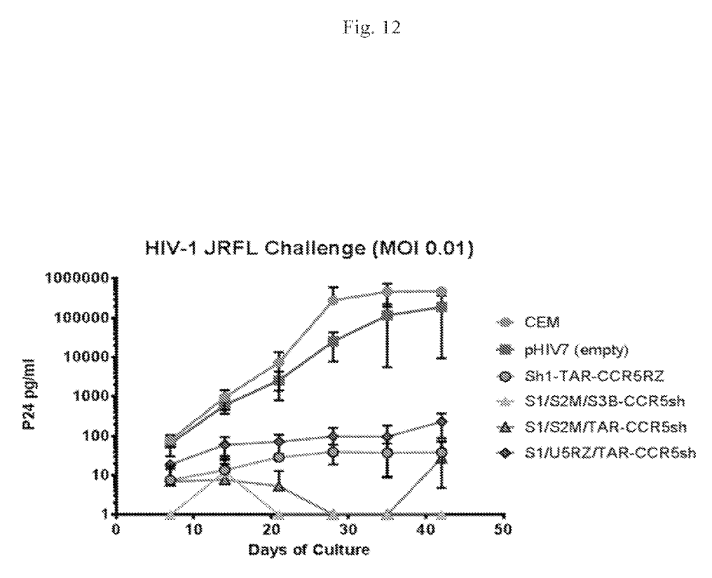

To validate proper processing and expression of all small RNA transgenes in this combination, Applicants transduced CEM T lymphocytes to create stably expressing cell line then performed northern blotting analysis (FIG. 11). To evaluate the antiviral potency, Applicants challenged the stably expressing CEM T-cells with M-tropic JR-FL strain of HIV virus and monitored viral replication for 42 days. Result showed MCM7 vectors have potent antiviral activity with similar activity to the first generation lentiviral construct (Sh1-TAR-CCR5RZ) with at least 3-logs reduction in viral replication (FIG. 12).

RNA Promoter 3/RNA5/Termination Seq 3/Transgene Orientation

To further increase the functionality and versatility of Applicants' MCM7 platform, Applicants incorporated a second independent small RNA expression cassette. This transgene is incorporated downstream of the MCM7 platform in the multiple cloning site before the protein promoter. This additional small RNA transgene is independently expressed from a Pol III promoter and therefore terminated by the Pol III termination sequence (a consecutive series of 5 to 6 uracil nucleotides). Examples of Pol III promoters include but not limited to U6, H1, tRNA^{Lys}, tRNA^{Ser}, tRNA^{Arg} and examples of small RNA transgene include but not limited to RNA decoys, RNA ribozymes, and siRNAs expressed either as a short hairpin RNA (shRNA) or as a precursor miRNA (pre-miRNA). SiRNAs targeting any gene of interest can be incorporated, including endogenous or viral genes. Furthermore, dual-targeting siRNA (i.e. bifunctional siRNAs) that target two separate genes or identical gene at two different locations can also be utilized. Applicants have successfully incorporated and expressed a Pol III U6-driven TAR RNA decoy (U16TAR) in this configuration.

### Example 2

### Lentiviral Vector Design to Incorporate a Polycinstronic MCM7 Platform and a Drug Selection Marker (MGMT^{P140K}) for Combinatorial RNA-Based Gene Therapy

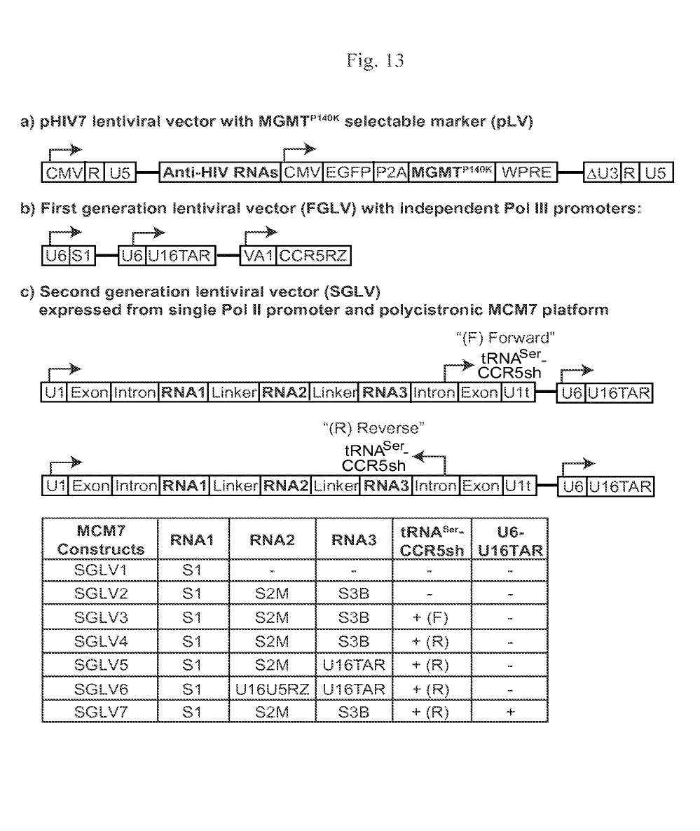

Lentiviral vectors are efficient gene delivery vehicles with the ability to transduce non-dividing cells such as HSPCs resulting in long-term expression of the therapeutic transgenes in differentiated progeny. Applicants modified a third generation, self inactivating lentiviral vector, pHIV7, that previously demonstrated high efficiency in transducing primary CD4+ T lymphocytes and HSPCs (Yam, P Y et al. (2002). Design of HIV vectors for efficient gene delivery into human hematopoietic cells. Molecular therapy: the journal of the American Society of Gene Therapy 5: 479-484) to also express MGMT^{P140K} from a CMV promoter (FIG. 13a). Applicants observed no differences in viral titer and transduction efficiency with inclusion of MGMT^{P140K} transgene (data not shown). Applicants' earliest lentiviral vector used independent Pol III promoters (FGLV, FIG. 13b) to ensure strong and persistent expression of antiviral small RNA transgenes. Subsequently, Applicants engineered an MCM7 platform that co-expresses three small RNAs within the polycistronic cluster from a single Pol II U1 promoter (SGLV, FIG. 13c) to express small RNAs at more moderate levels to reduce potential vector toxicity. The MCM7 platofrm is designed to co-express three small RNAs within the polycistronic cluster using a single Pol II promoter. Although any Pol II promoter can be utilized to engineer tissue-specific transgene expression in this platform, Applicants selected the U1 promoter for ubiquitous and persistent transgene expression in all hematopoietic cells derived from HPSCs. Applicants previously demonstrated combinations of both si- and snoRNAs can be multiplexed in this format with antiviral functionality (Chung, J, et al. (2012). Endogenous MCM7 microRNA cluster as a novel platform to multiplex small interfering and nucleolar RNAs for combinational HIV-1 gene therapy. Human gene therapy 23: 1200-1208). Since the MCM7 platform expresses small RNAs at lower levels than independent Pol III promoters, Applicants reasoned that Applicants could incorporate additional small RNA transgenes to further enhance antiviral potency without a significant increase in toxicity. Applicants incorporated a CCR5-targeting siRNA driven by an independent Pol III transfer RNA Serine promoter (tRNA^{Ser}) [tRNA^{Ser}-CCR5sh cassette] in the 3′ intron of MCM7 in both orientations [forward (SGLV3) or reverse (SGLV4), FIG. 13c] as a viral entry inhibitor. Applicants also introduced a fifth RNA cassette, the nucleolar TAR RNA decoy driven by the independent Pol III U6 promoter (U6-U1 6TAR cassette) to increase antiviral potency by inhibiting Tat-dependent viral transcription. This cassette was cloned outside of the MCM7 transgene to reduce the possibility of promoter interference that could negatively impact gene expression. These novel lentiviral vectors express up to five antiviral small RNAs to block both viral entry and replication of both tropisms of HIV with cassettes driven by both Pol II and III promoters.

Biological Activity and Expression Optimization of the tRNA^{Ser}-CCR5sh Cassette in the MCM7 Platform

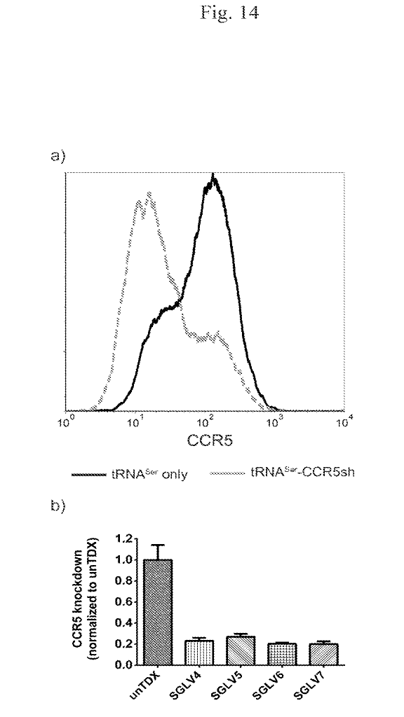

Applicants investigated the use of transfer RNA (tRNA) promoters to express candidate antiviral RNAs due to their small size, ease in multiplexing and independent regulation of RNA expression. T his (Pol III) expression strategy utilizes the endogenous transfer RNA biogenesis pathway to express a primary tRNA-shRNA chimeric transcript and then release the mature shRNA by tRNAse Z cleavage (Scherer, L J, et al. (2007). Optimization and characterization of tRNA-shRNA expression constructs. Nucleic acids research 35: 2620-2628) for further processing into mature siRNA. In the present constructs, Applicants utilized a Serine tRNA promoter (tRNA^{Ser}) to express a CCR5 shRNA as an entry inhibitor against R5-tropic HIV. To assess the ability of this construct to down regulate surface CCR5 expression, Applicants transiently transfected plasmids containing either the promoter sequence only (the endogenous tRNA^{Ser} gene) or with the tRNA^{Ser}-CCR5sh cassette into a CCR5 over-expressing U373-MAGI-CCR5E cells (Vodicka, M A, et al. (1997). Indicator cell lines for detection of primary strains of human and simian immunodeficiency viruses. Virology 233: 193-198). The reduction in CCR5 expression by flow cytometry was evaluated 72 hours after introduction of the construct. Potent and specific CCR5 knockdown was only observed with tRNA^{Ser}-CCR5sh cassette and not with the (empty) tRNA^{Ser} promoter alone (FIG. 14a). Applicants also utilized quantitative RT-PCR for CCR5 RNA expression as the measure of functionality of Applicants' tRNA^{Ser}-CCR5sh cassette in primary cells where CCR5 protein expression is often difficult to resolve by flow cytometry. Applicants observed 80% down regulation of CCR5 transcript RNA in primary macrophages derived from in vitro differentiation of gene modified HSPCs. This data demonstrates that sufficient CCR5 siRNA is produced in the context of MCM7 platform to down regulate gene expression in primary cells (FIG. 14b).

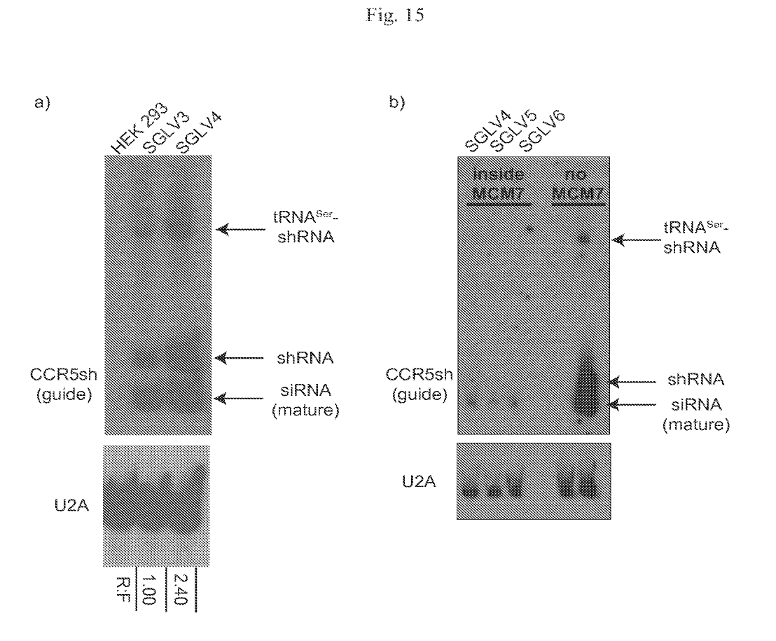

To further assess the processing of the tRNA^{Ser}-CCR5sh cassette in combination with the other anti-HIV elements, Applicants incorporated this expression unit into the 3′ intron of the MCM7 platform (“inside MCM7”) in both orientations [forward (SGLV3) and reverse (SGLV4), FIG. 13c] with respect to the parental U1 promoter. When transiently transfected into HEK 293 cells, Applicants observed the reverse orientation (SGLV4) gives 2.4-fold enhancement in transgene expression based on Northern blotting analysis with a radioactive probe specific for detecting the guide strand of the CCR5 shRNA (FIG. 15a). Moreover, this analysis further distinguishes products in various stages of processing including the primary tRNA^{Ser}-shRNA chimeric transcript, the released shRNA, and the mature Dicer-processed siRNA sequence capable of gene silencing. The siRNA is the dominate product suggesting efficient processing but some saturation of the processing pathway due to over-expression from the transient transfection is also evident (FIG. 15a). Interestingly, pHIV7 containing only the tRNA^{Ser}-CCR5sh RNA cassette (no MCM7) drives dramatically higher expression levels in stably transduced CEM T lymphocytes than the above vector where the transgenes is inside the MCM7 3′ intron based on Northern blotting analysis (FIG. 15b), even though the vector is present at only one to two integrated copies per cell. These high levels of tRNA^{Ser}-CCR5shRNA expression result in the accumulation of incompletely processed tRNA^{Ser}-shRNA chimeric transcript and large quantities of shRNAs. Based on these results, Applicants concluded the tRNA^{Ser}-CCR5shRNA cassette in reverse orientation located within the 3′ intron of MCM7 platform would provide a more optimal level of siRNA expression.

Flexible MCM7 Platform Expresses Up to Five Antiviral Small RNAs at Physiological Level with Efficient Processing

Combinatorial therapy has inherent challenges including various types of interference or competition between the individual elements that can negatively impact the potential therapeutic outcome. For example, when a vector encodes multiple RNAi triggers, competition can arise between different RNAi triggers for RNAi pathway components and incorporation into the RNA-induced silencing complex (RISC). To ensure all the transgenes are expressed and processed into functional forms, Applicants performed Northern blotting analysis with CEM T lymphocytes that stably express the transgenes. To ensure the expression level resembles the optimum physiological condition of one to two copies of integrated vectors in HSPCs, Applicants only sorted transduced populations that were lower than 30% EGFP positive (EGFP+).

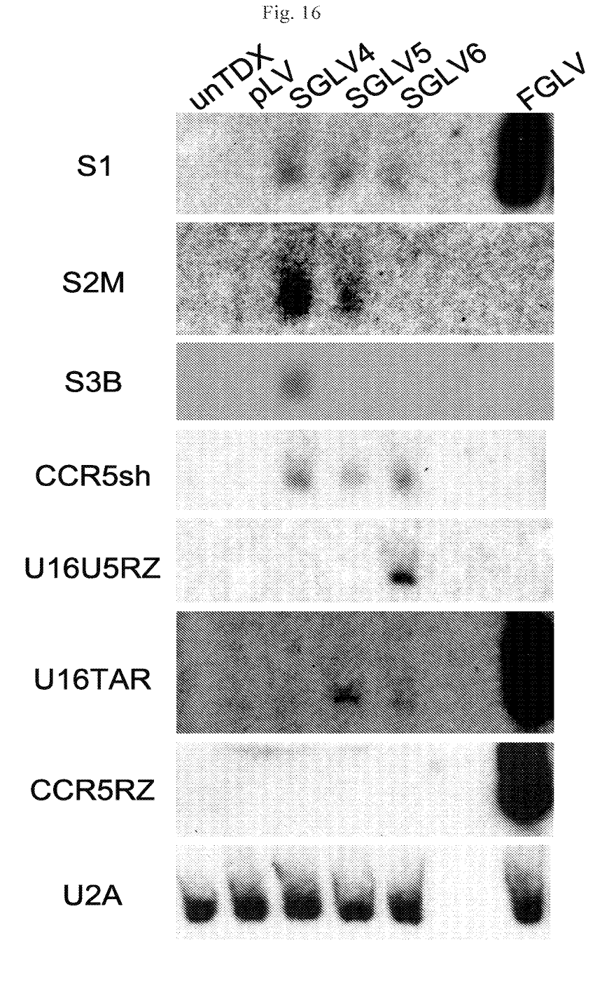

Northern expression analyses indicate that incorporation of additional RNA cassettes such as tRNA^{Ser}-CCR5sh did not interfere with expression and processing of other transgenes in the MCM7 platform (FIG. 16). Applicants also confirmed correct and efficient processing of all RNAi triggers precursors (51, S2M, S3B, and CCR5sh) into functional mature 20-23 nt siRNAs, and the small nucleolar RNAs (U16U5RZ and U16TAR) into 132-nt processed products (FIG. 16). Applicants did not observe accumulation of pri- and pre-miRNA precursors suggesting that the endogenous RNAi pathway is not saturated and that it is possible to co-express four siRNAs safely in a combinatorial approach. Therefore, MCM7 serves as a versatile tool in a multiplexing approach with the capacity for incorporating small RNA cassettes driven by both Pol II and Pol III RNA promoters. Comparing transgene expression levels between the newer second generation and former first generation combinatorial strategies with two different classes of RNA promoters, SGLVs with Pol II driven MCM7 platform express transgenes at a lower level compared to expression from FGLV with independent Pol III promoters (lanes 3-5 versus lane 6 in FIG. 16). While the lower levels resulting from the U1 promoter may reduce toxicity, Applicants questioned whether these levels were sufficient for efficacy. Therefore, Applicants next turned to assessing the anti-HIV activity of the current combinations.

The MCM7 Platform Produces Sufficient Small RNAs to Protect Gene Modified Cells from R5 Tropic HIV

Applicants first assessed the antiviral functionality by viral challenge of gene modified CEM T lymphocytes with R5 tropic HIV-1 JR-FL for 42 days, monitoring viral replication by p24 capsid levels in the culture supernatant (FIG. 12). All the therapeutic constructs (FGLV, SGLV4, SGLV5, and SGLV6) provided long-term protection, with up to a 5-log reduction in p24 capsid production in comparison to pLV and untransduced (unprotected) cells. Notably, SGLV4 and SGLV5 had almost no detectable levels of p24 capsid during this long term challenge, providing evidence that the MCMI platform indeed expresses sufficient amount of small RNAs for functionality. To further demonstrate the feasibility of stem cell based approach in protecting gene modified progenies, Applicants evaluated antiviral potency of Applicants' constructs using macrophages derived from HSPCs. Macrophages are myeloid cells particularly suited for demonstrating anti-HIV potency in HIV challenge assays with vectors expressing CCR5 RNAi triggers because of the requirement of CCR5 co-receptor usage for infection with R5-tropic virus. Applicants wished to design an assay where adult CD34+ HPSCs are the substrate for gene modification as they are the eventual clinical target cell population for a stem cell based gene therapy approach. After transduction, CD34+ HPSCs can be induced into differentiating into the myeloid cells in vitro and efficacy of the candidate constructs evaluated by challenge with R5-tropic Bal HIV virus. To this end, Applicants developed a novel single cell flow cytometric assay of intracellular staining with an antibody specific to HIV-1 core antigens (55, 39, 33, and 24 kD proteins) to monitor viral replication. The 55 kD protein is the primary precursor, while the 39 and 33 kD proteins are the intermediates of the mature 24 kD core protein (Chassagne, J, et al. (1986). A monoclonal antibody against LAV gag precursor: use for viral protein analysis and antigenic expression in infected cells. Journal of immunology 136: 1442-1445). Applicants used uninfected macrophages to establish non-specific staining and background signal in the flow cytometric assay. Using this novel method, Applicants were able to follow the kinetics of HIV infection on the cellular basis during the 42-day challenge in primary macrophages.

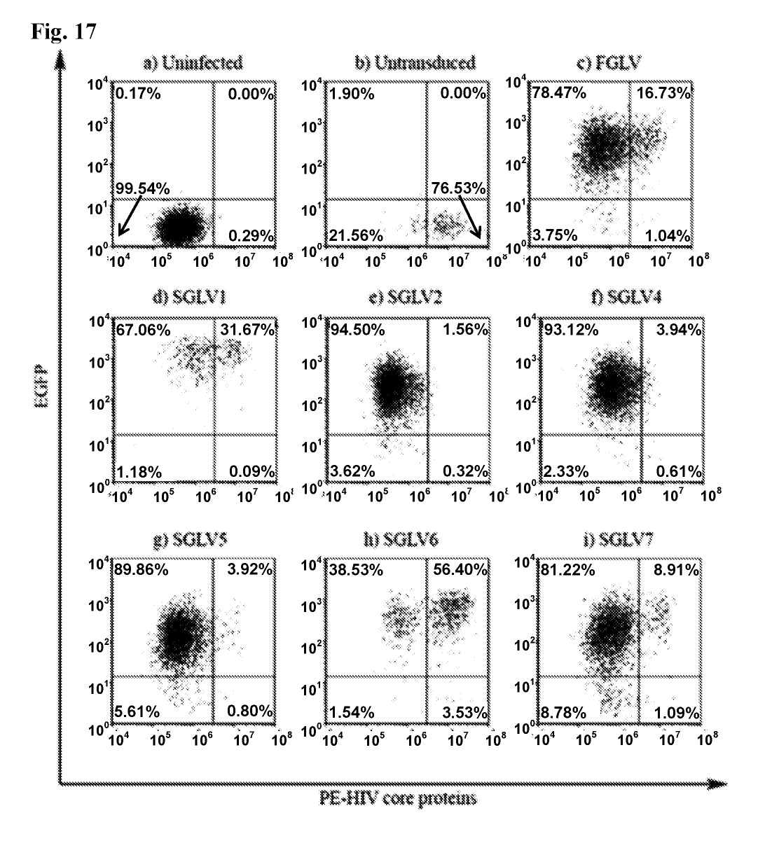

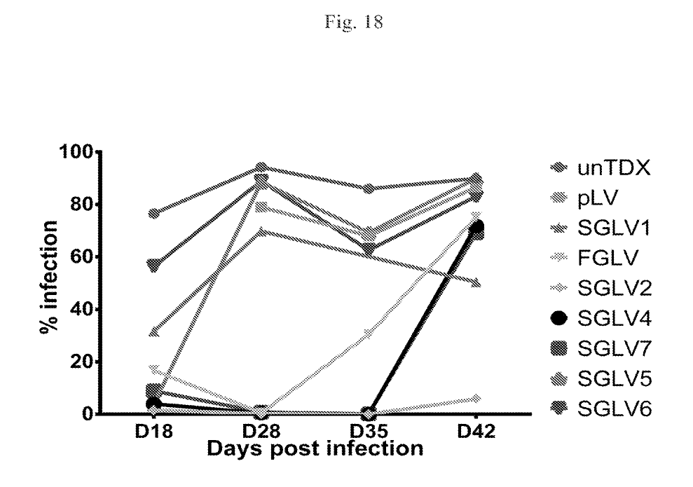

Representative intracellular HIV staining results at D18 of the viral challenge are shown in FIG. 17. Applicants observed a 2-log difference in fluorescence intensity comparing unspecific background staining of uninfected cells (FIG. 17a) to an actual HIV infected culture (FIG. 17b) validating that Applicants' novel flow cytometric assay has excellent sensitivity for HIV detection. Furthermore, Applicants found that 76.5% of untransduced (unprotected) macrophages infected with HIV at D18 (FIG. 17b) and as high as 95% at later time points (e.g., D28) (FIG. 18, unTDX trace) providing good dynamic range as a vector screen to distinguish constructs with varying antiviral activities. For example, this flow cytometric assay can distinguish constructs with intermediate protection [31.7% HIV infection in SGLV1 (FIG. 17d) and 56.4% in SGLV6 (FIG. 17h)] from constructs with the best protection [16.7% HIV infection in FGLV (FIG. 17c); 1.6% in SGLV2 (FIG. 17e); 3.9% in SGLV4 (FIG. 17f); 3.9% in SGLV5 (FIG. 17g); 8.9% in SGLV7 (FIG. 17i)]. When comparing constructs with the highest antiviral activities, Applicants observed that SGLV2 had better antiviral activity (1.6% infection, FIG. 17e) compared to small RNAs expressed from independent strong Pol III promoters such as in FGLV (16.7% infection, FIG. 17c). There results emphasize that the higher levels of small RNA transgene expression are not necessary for anti-HIV potency, as lower RNA expression from the MCM7 platform with a single Pol II U1 promoter is sufficient for functionality. When comparing different RNA combinations in the MCM7 platform (SGLVs), Applicants observed that the antiviral protection also increase in constructs containing at least 2 anti-HIV siRNAs (i.e., SGLV4 and SGLV5). This could be due to the additive nature and efficiency of multiple siRNAs in cleaving HIV targets to inhibit HIV replication.

To Applicants' surprise, Applicants observed no benefit in adding the tRNA^{Ser}-CCR5sh cassette as an entry inhibitor (3.9% in SGLV4, FIG. 17f) or the nucleolar TAR RNA decoy (8.9% in SGLV7, FIG. 17i) to the parental SGLV2 construct (1.6% in FIG. 17e), perhaps because the parental construct already has potent antiviral activity with very low HIV infection. Another measure of the potency of the various constructs is the duration of protection from HIV-1 replication. Applicants therefore followed the kinetics of HIV infection for 42 days with the aforementioned intracellular HIV staining methodology to identify construct(s) with persistent protection (FIG. 18). In this group, Applicants observed an increase in the frequency of infected cells in some constructs over time: SGLV5 (D28)>FGLV (D35)>SGLV4=SGLV7 (D35), but SGLV2 had no significant increase in viral infection though 42 days of culture. Applicants' long term results further confirm that over-expression of small RNA is not a guarantee of long term antiviral protection and that the MCM7 platform does provide lower but sufficient level of RNA expression for functionality.

Lower Small RNA Expression Reduces the Possibility of Potential Vector-Related Toxicity

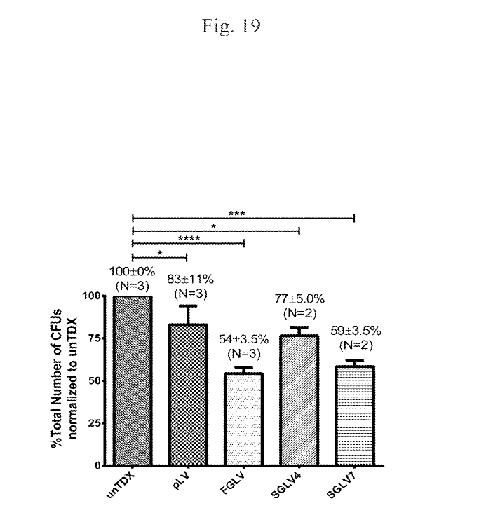

Having established that sufficient levels of small RNAs are produced from the MCM7 platform for potent antiviral protection, Applicants continued to explore the issue of potential vector-related toxicity on hematopoietic potential using an in vitro colony forming unit (CFU) assay. The CFU assay measures the hematopoietic potential of uni- and multi-potent myelo-erythroid progenitors and therefore can be used to assess potential toxicity of multiplexed RNA expression in gene modified HSPCs. Applicants normalized the total number of CFUs to the untransduced control with respect to each donor to account for differences in hematopoietic potential between donors. Applicants found the transduction process for gene modification and the empty vector had minimal impact on hematopoietic potential (83±11% for pLV, FIG. 19) which is relevant for clinical translation of stem cell based gene therapy. In contrast, the levels of therapeutic small RNA expressed from independent strong Pol III promoters in FGLV had a negative impact on hematopoietic potential (54±4%, FIG. 19). However, when the small RNA expression is lowered with the MCMI platform (e.g., SGLV4), Applicants observed a recovery of hematopoietic potential to a similar level as the empty vector (77±5% vs. 83±5%, respectively, FIG. 19). Surprisingly, incorporating U6-U16TAR cassette that expresses the nucleolar TAR RNA decoy (SGLV7) created a sharp decline in CFU formation (from 77±5% to 59±4%, FIG. 19). It is therefore possible that the U6-U16TAR RNA cassette alone may be responsible for the reduction in hematopoietic potential in the independent Pol III driven FGLV observed in this assay. Further experiments, including the assessment of each individual RNA cassette on CFU potential is required to establish the relative roles of the higher levels of the RNA antiviral expression levels versus the specific inclusion of the U6-U16TAR moiety on the loss CFU potential observed with FGLV.

In Vivo O⁶-BG/BCNU Drug Selection for Enrichment of Gene Modified Cells in Humanized NSG Mice

One of the current hurdles in stem cell based gene therapy for HIV is the low frequency of gene modified cells, typically generated by current in vivo protocols, preventing clinical assessment of antiviral efficacy. Therefore, Applicants wished to include a gene in Applicants' second generation lentiviral vectors that confers a survival advantage to a clinically relevant drug, thereby increasing the frequency of HIV resistant cells in vivo. The endogenous MGMT enzyme is responsible for repairing DNA damage caused by alkylating agents such as BCNU. O⁶-BG deactivates endogenous MGMT so that cells cannot repair BCNU-induced DNA damages resulting in cell death. However, gene modified cells that express a modified MGMT (MGMT^{P140K}) are not sensitive to O⁶-BG treatment and therefore can repair DNA damage from BCNU treatment and survive. The net result of the O⁶-BG/BCNU selection is increased frequency of gene modified cells. Therefore, Applicants included this drug resistance gene in Applicants' anti-HIV constructs and developed a protocol to test for enrichment of gene modified HSPC and CD4+ progeny in vivo. In order to establish that a sufficient level of MGMT^{P140K} was expressed in the relevant cell types, CD34+ HSPCs were transduced with an MGMT^{P140K} expressing vector (FGLV) and used to transplant immunodeficient NSG mice as described in Experimental Procedures. Animals were treated with O⁶-BG and BCNU at 7^{th} and 8^{th} weeks (2× treatment cohort) or at 7^{th}, 8^{th} and 9^{th} weeks (3× treatment cohort) following transplantation. Two or three weeks after completion of the O⁶-BG/BCNU treatment (i.e., 11 weeks following transplantation), animals were necropsied and the level of engraftment and frequency of gene modified cells in the spleen and bone marrow were evaluated.

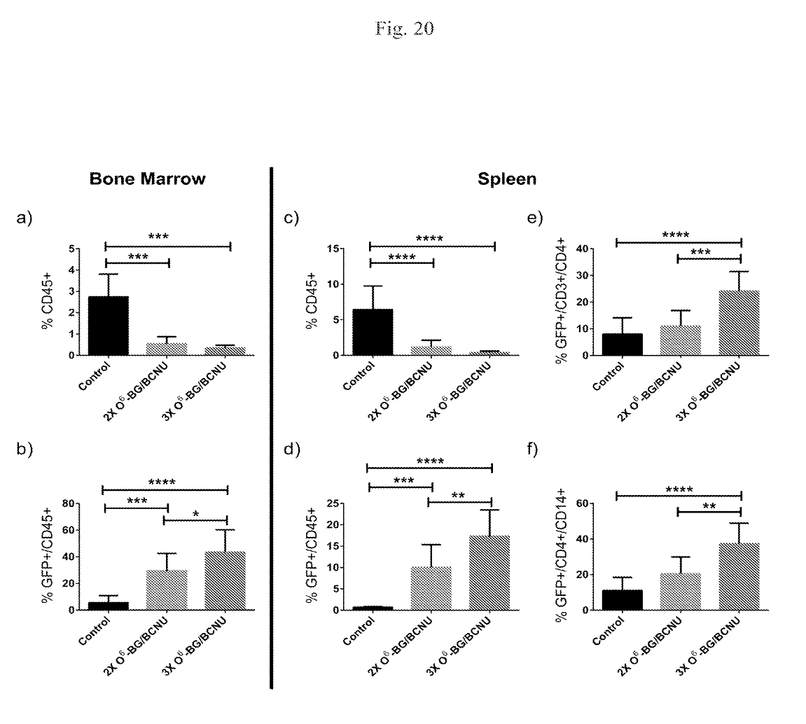

Applicants' results demonstrate that engraftment of the bone marrow and spleen with human (CD45+) cells was significantly reduced in treated animal cohorts relative to the control cohort (p<0.001) (FIGS. 20a and 20c, respectively) but the frequency of GFP+/CD45+ cells in the bone marrow and spleen was enriched 10 and 15-fold in the 2× and 3× treated cohorts respectively (FIGS. 20b and 20d). The average frequency of GFP+/CD3+/CD4+ T lymphocytes in the spleen increased 3-fold only when O⁶-BG/BCNU treatment was performed three times (FIG. 20e). Similarly, GFP+/CD4+/CD14+ monocytes were increased 3-fold in the spleens of mice following three O⁶-BG/BCNU treatments (FIG. 200. No deficiencies in lineage development were noted in the drug treated mice (data not shown). These data demonstrate that selective enrichment of HIV-target cells occurred in vivo and thus may allow for post-transplant increases in the frequency of HIV-resistant cells in patients where engraftment of these cells may be suboptimal.

Applicants demonstrate here that the MCM7 intron is a flexible and versatile platform for co-expressing combinations of up to three si- and snoRNAs within the MCM7 sequence and the ability to add additional independent RNA cassettes (both inside and outside of MCM7) for a total of five small RNAs. To Applicants' knowledge, this is the first example of multiplexing both classes of RNA promoters in a combinatorial approach. Applicants observed expression and complete processing of all small RNA transgenes into functional forms without saturation of processing pathways or promoter interference that could negatively impact transcription. Interestingly, although the tRNA^{Ser}-CCR5sh cassette is expressed at high levels in the pLV without MCM7, its expression is much weaker in the context of MCM7. This result may be related to the requirement of splicing of the intronic MCM7 cluster prior to transgene expression. Moreover, expression is also related to orientation of the cassette in MCM7 highlighting the importance of optimal placement in multiplexing strategies. The level of small RNA expression from the MCM7 platform driven by a single Pol II U1 promoter is much weaker compared to the amounts driven by independent Pol III promoters. Nonetheless, Applicants established that sufficient amounts of antiviral small RNA were produced from the MCM7 platform to protect gene modified CEM T lymphocytes and primary macrophages derived from gene modified HSPCs from R5 tropic HIV, with the duration and level of protection highly dependent on RNA combination. This suggests that the optimal level of RNA expression is more important than achieving maximal levels of RNA expression for a given modality. It is likely that the overall amount of small RNA processing that can occur in a cell at any given time is limited. In theory, because of the catalytic nature of siRNAs and ribozymes in turning over multiple substrates, it should take less to achieve the same therapeutic effect compared to agents that sequester their target in stoichoimetric ratio such as TAR RNA decoys.

Applicants' results show that the multiple siRNAs in the MCM7 platform provided better protection against HIV, e.g., three siRNAs targeting tat and rev (SGLV2) was better than single tat/rev siRNA (SGLV1). This could be due to the additive nature and efficiency of multiple siRNAs in cleaving HIV targets, while increasing the selective pressure against viral escape. Interestingly, the addition of two other potent anti-HIV RNAs, tRNA′^{r} CCR5sh entry inhibitor and a U6-driven nucleolar TAR RNA decoy (SGLV4 and SGLV7, respectively), to the parental triple siRNA construct (SGLV2) did not provide additional benefit in inhibiting viral replication or preventing viral breakthrough. In fact, the additional small RNA cassettes actually negatively impacted potency with the observation of viral breakthrough at 35 days with the parental construct observed limited breakthrough through the 42 day culture. It is unclear why addition of the entry inhibitor and the nucleolar TAR RNA decoy was less optimal in inhibiting HIV, although it is possible that CCR5 siRNA may compete with other anti-HIV siRNAs (i.e., S1, S2M, S3B siRNAs) for RISC factors in gene silencing. Furthermore, TAR RNA has been reported as a pri-miRNA that is processed into functional miRNAs (Ouellet, et al. (2008). Identification of functional microRNAs released through asymmetrical processing of HIV-1 TAR element. Nucleic acids research 36: 2353-2365) and a potent binder to TRBP (HIV-1 TAR RNA binding protein) within the endogenous RISC that negatively impacts RNAi pathway. The newly discovery of toxicity associated with over-expression of the TAR RNA decoy (see below) could implicate selection of cells with overall less RNA expression translating into less optimal antiviral protection.

Applicants performed an in vitro CFU assay to identify any vector-related myelo-erythroid toxicity. Applicants' data demonstrates that the MCM7 platform has the capacity to safely express four siRNAs with complete processing without reducing in vitro hematopoietic potential of gene modified HSPCs. Surprisingly, Applicants observed a reduction of CFU potential with incorporation of additional U6-U16TAR cassette making the first report of toxicity related to over-expression of RNA decoys. It is possible that the U6-U16TAR cassette is responsible for the observed toxicity in FGLV, but further experiments including assessment of each individual Pol III RNA cassette on CFU potential are likely required. Additional studies may be required to assess the potential for toxicity in other (lymphoid) cell populations not addressed in these in vitro assays. Increasing the frequency of gene modified cells in vivo may be required to achieve a clinically meaningful antiviral effect in cases where engraftment of gene modified cells is low. Therefore, Applicants incorporated a drug resistance gene (MGMT^{P140K}) in Applicants' improved lentiviral vector to allow for in vivo enrichment of gene modified cells using alkylating agents. Applicants' result demonstrated the feasibility of this approach in increasing the frequency of gene modified CD4+ cells using in vivo treatment with levels of BCNU similar to those used in a non-human primate model of HIV gene therapy (Younan, P M, et al. (2013). Positive selection of mC46-expressing CD4+ T cells and maintenance of virus specific immunity in a primate AIDS model. Blood 122: 179-187). In summary, combinatorial gene therapy approaches are often most effective when targeting multiple stages of viral replication. Applicants present here the improved MCMI platform with enhanced flexibility, increased safety with sufficient level of transgene to long term inhibition of HIV in CD34-derived macrophages as a useful tool for combinatorial gene therapy. Applicants demonstrate that more is not always better but rather a balance between transgene expression, mechanism of action, and target choice is required for optimizing the combinatorial approach.

### Example 3

NSG mice (N=8 per group) were transplanted with 0.5×10⁶ CD34+ HSPC that had been transduced with either Applicants' first generation lentiviral vector (FGLV) or second generation lentiviral vectors SGLV2 and SGLV4 (See table 7 for construct identity). Prior to transplantation the CD34+ HSPC were transduced at 28% (FGLV), 44% (SGLV2) and 31% (SGLV4) as determined by flow cytometric analysis for GFP 5 days after transduction. Animals were maintained according to IACUC protocols and administered 20 μg/week/mouse Fc/IL-7 to enhance T-cell development as previously reported.

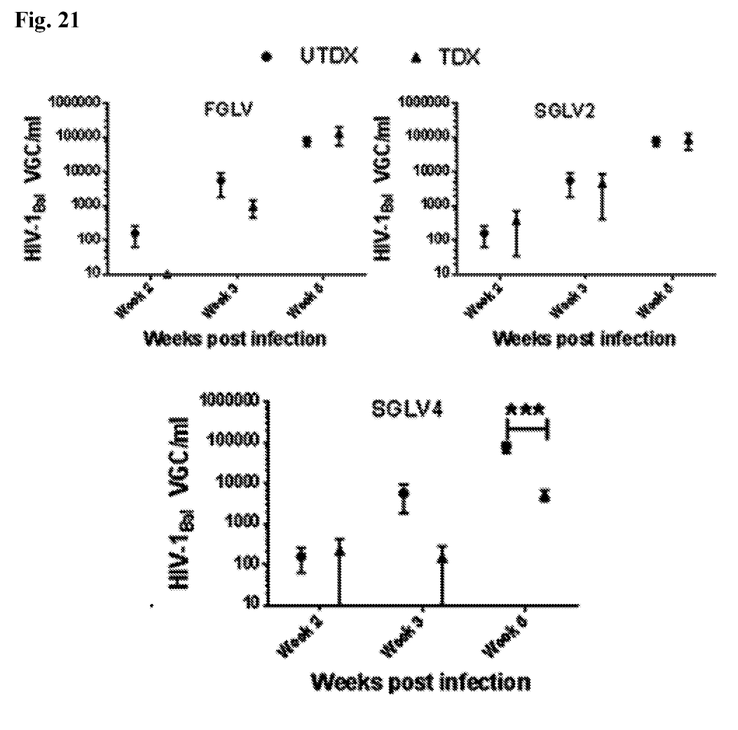

Eleven weeks after transplantation, animals were challenged with HIV-1_{Bal} virus (41580 IU/mouse) and mice were followed for serum viremia every two weeks by RT-PCR. Some mice died during the 5 weeks following HIV challenge for undetermined reasons and so all animals were necroposied 6 weeks after infection. When comparing the level of HIV in the serum of mice transplanted with untransduced or transduced HSPC, serum viremia was reduced >1 log by SGLV4 at 5 weeks after infection (FIG. 21).

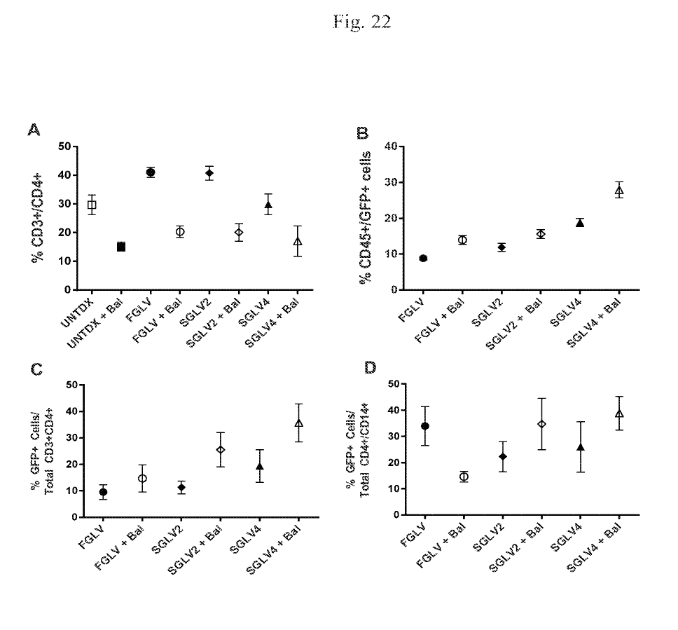

Applicants evaluated the percentage of CD45+CD4+, CD45+/GFP+, CD45+/CD3+/CD4+/GFP+ and CD45+/CD14+/GFP+ cells in the spleen of each animal at necropsy. While Applicants observed a reduction in the overall number of CD4+ T-cells, all animals transplanted with gene modified HSPC showed an increased in the average level of CD45+/GFP+ cells in the spleen. All animals transplanted with gene modified HSPC also showed increases in the CD3+/CD4+/GFP+ cells among all CD3+/CD4+ T-cells and SGLV2 and SGLV4 showed an increase in the average level of CD4+/CD14+/GFP+ monocytes among all CD4+/CD14+ monocytes (FIG. 22).

These results demonstrate that Applicants have created an effective anti-viral construct that can both control viremia and confer a selective advantage to gene modified T-cell and monocytes in the face of R5 viral infection. The candidate construct chosen from these experiments is SGLV4 which shows greater ability to control virus in both in vitro and in vivo cultures and is less toxic than Applicants' previous clinical construct Applicants have successfully developed and utilized an adult model of HIV infection that will allow Applicants to test these vectors in samples from HIV+ patients and determine Applicants' ability to create clinical products.

### Experimental Procedures

### Generation of MCM7 snoRNA Constructs

The U16RBE and U16U5RZ snoRNA molecules were amplified by PCR from pTZ/U6-U16RBE and pTZ/U6-C36U5 DNA vectors (Michienzi, A. et al., AIDS Res Ther., 3, 13 (2006); Unwalla, H. J. et al., Mol Ther., 16, 1113-1119 (2008)), using primer sets A, and B, as XhoI/HindIII and EcoRI/BamHI fragments, respectively. The fragments were digested with appropriate enzymes followed by cloning into the pcDNA3-CMV-MCM7-S1/S2/U16TAR plasmid (Aagaard, L. A. et al., Gene Ther., 15, 1536-1549 (2008)). The U1-specific transcriptional terminator along with a new Nod site was introduced at the terminus of the common 3′ region of the MCM7 cassette by replacing the original DNA sequence with a PCR product generated with primer set C. The CMV promoter was replaced by the U1 promoter flanked by MluI and KpnI sites generated from amplification from a U1 plasmid (pKS-U1, unpublished data) with primer set D.

To generate lentiviral vectors, the U1-MCM7-Ult fragments were excised by MluI and Nod digestion and ligated into the pHIV7-EGFP lentiviral vector in both forward and reverse orientations (i.e., the U1 promoter is in the same or opposite orientation as the packaging CMV promoter, respectively) depending on the directionality of the multiple cloning site.

Primer sequences are given below with restriction sites underlined and U1-specific terminator in bold:

A:
Forward:
(SEQ ID NO: 1)
5′-CCC CCC CCTC GAG CTT GCA ATG ATG TCG TAA
TTT G-3′
Reverse:
SEQ ID NO: 2)
5′-CCC CAA GCT TAA AAA TTT CTT GCT CAG TAA
GAA TTT-3′
B:
Forward:
SEQ ID NO: 3)
5′-CCC CCC CGA ATT CCT TGC AAT GAT GTC GTA
ATT TG-3′
Reverse:
SEQ ID NO: 4)
5′-CCC CGG ATC CAA AAA TTT CTT GCT CAG TAA
GAA TTT-3′
C:
Forward:
SEQ ID NO: 5)
5′-ATC GAT CCG CGG ATG CTG GGG GGA GGG GGG
AT-3′
Reverse:
SEQ ID NO: 6)
5′-ACG TGT TAA CGC GGC CGC AGT CTA CTT TTG
AAA CTC TGC CCC TTG TCT CCT AGA-3′
D:
Forward:
SEQ ID NO: 7)
5′-ATC GAT ACG CGT CTA AGG ACC AGC TTC TTT
GGG AGA G-3′
Reverse:
SEQ ID NO: 8)
5′-ATC GAT GGT ACC GAT CTT CGG GCT CTG CCC
CG-3′

Sequence of tRNASer-CCR5shRNA Constructs:

tRNA^{Ser} promoter sequence (5′ leader sequence
in bold, promoter sequence in italic):
(SEQ ID NO: 9)
5′ GAAAATGACTTTGCCACGCTTAGCATGTGACGAGGTGGCCGAGTGGT
TAAGGCGATGGACTGCTAATCCATTGTGCTCTGCACGCGTGGGTTCGAAT
CCCATCCTCGTCG 3′
Bifunctional siRNA (bi-CCR5-5) as pre-miRNA
sequence:
(SEQ ID NO: 10)
5′ GGCCTGGGAGACCTGGGGACGCTGTGACACTTCAAACTTCCCCAGCT
CTCCCAGGCCCG 3′
Bifunctional siRNA (bi-CCR5-5) as shRNA
sequence:
(SEQ ID NO: 11)
5′ GCCTGGGAGAGCTGGGGAATTCAAGAGATTCCCCAGCTCTCCCAGGC
3′
CCR5-12sh sequence:
(SEQ ID NO: 12)
5′ AGTGTCAAGTCCAATCTATGATTCAAGAGATCATAGATTGGACTTGA
CAC 3′

Generation of Improved Second Generation Lentiviral Vectors with Polycistronic MCM7 Platform and Selectable MGMT^{P140K} Marker

The MGMT^{P140K} transgene was co-expressed with the EGFP marker from a polycistronic message utilizing a self-cleaving P2A peptide sequence from the CMV promoter.

The MGMT^{P140K} gene was first PCR amplified from pRSC-SMPGW2 plasmid (Trobridge, G D, et al. (2009). Protection of stem cell-derived lymphocytes in a primate AIDS gene therapy model after in vivo selection. PloS one 4: e7693), using the following primers (Forward: 5′-GGG TCT AGA ATG GAC AAG GAT TGT GAA ATG AAA CGC-3′ [SEQ ID NO:13] and Reverse: 5′-GGG GAA TTC CGT ACG TCA GTT TCG GCC AGC AGG CG-3′ [SEQ ID NO:14]) flanked by XbaI and EcoRI sites. The fragment was digested with appropriate enzymes then subcloned into NheI and EcoRI sites of the pFUG-P2A-WPRE vector (John Burnett, unpublished) just downstream of the P2A peptide sequence to generate pFUG-P2A-MGMT^{P140K}-WPRE. The P2A-MGMT^{P140K} fragment was then excised by BsrGI and BsiWI digestion then subclone into the BsrGI site of pHIV7-GFP vector (Yam, P Y, et al. (2002). Design of HIV vectors for efficient gene delivery into human hematopoietic cells. Molecular therapy: the journal of the American Society of Gene Therapy 5: 479-484) to generate a modified lentiviral vector pHIV7-GFP-P2A-MGMT^{P140K}. The same strategy was utilized to introduce the P2A-MGMT^{P140K} fragment into FGLV (Li, M J, et al. (2005). Long-term inhibition of HIV-1 infection in primary hematopoietic cells by lentiviral vector delivery of a triple combination of anti-HIV shRNA, anti-CCR5 ribozyme, and a nucleolar-localizing TAR decoy. Molecular therapy: the journal of the American Society of Gene Therapy 12: 900-909) to generate the modified lentiviral vector suitable for drug selection. The CCR5-targeting siRNA is expressed from the Pol III tRNA^{Ser} promoter. The tRNA^{Ser}-CCR5sh cassette was amplified from p1133-2 with the following primers (Forward: 5′-ATGC GCCGGC ATCGAT GAA AAT GAC TTT GCC ACG CTT AGC ATG TGA CGA GGT GGC CGA GT-3′ [SEQ ID NO:15] and Reverse: ATGC GGCGCC ATTTAAAT AAA AAA GTG TCA AGT CCA ATC TAT GAT CTC TTG AAT CAT AGA-3′ [SEQ ID NO:16]) flanked by NaeI and SwaI sites on the 5′ end and NarI and ClaI sites on the 3′ end. The NaeI-NarI and SwaI-ClaI fragments were subcloned into pcDNA3-U1-MCM7-Ult plasmids (Chung, J et al. (2012). Endogenous MCM7 microRNA cluster as a novel platform to multiplex small interfering and nucleolar RNAs for combinational HIV-1 gene therapy. Human gene therapy 23: 1200-1208) containing three triple combinations of small anti-HIV RNAs with the ClaI and SwaI sites approximately 70 bases upstream of the 3′ splice signal to generate clones in both forward and reverse orientations, respectively [pcDNA3-U1-MCM7-CCR5sh-Ult]. U1-MCM7-CCR5sh-Ult fragments were exercised from pcDNA3-U1-MCM7-CCR5sh-Ult plasmids with NruI and NotI digestion and subcloned into pHIV7-GFP-P2A-MGMT^{P140K} vector to generate SGLVs.

SGLV7 was generated by inserting the U6-U16TAR fragment into SGLV4. The U6-U16TAR fragment was generated from linearization of the pTZ/U6-U16TAR plasmid (Michienzi, et al. (2002). A nucleolar TAR decoy inhibitor of HIV-1 replication. Proceedings of the National Academy of Sciences of the United States of America 99: 14047-14052) by BamHI digestion, followed by fill-in of the overhang by Klenow Fragment to create a blunt end, then digested with SphI. This fragment was then ligated into SGLV4 that had been similarly treated except with the initial linearization with NotI enzyme.

Lentiviral Vector Production

Lentiviral vectors with appropriate inserts were packaged in 293T cells using calcium phosphate precipitation as previously described (Li, M. J., and Rossi, J. J., Methods Enzymol., 392, 218-226 (2005b) with the addition of 1.5 μg of pAgo2sh plasmid (Harris Soifer, unpublished) that expresses a short hairpin RNA (shRNA) transcribed from the human U6 promoter to down-regulate Argonaute 2 (Ago2) protein expression to reduce post-transcriptional gene silencing induced by anti-HIV siRNA within constructs during packaging. The viral supernatants were collected 48 hours post-transfection, concentrated via ultracentrifugation, and stored at −80° C. until use. Viral titers were determined by transduction of HT1080 cells and analyzed for EGFP expression with flow cytometry. In embodiments, 15 μg of transfer plasmid were co-transfected with helper plasmids (15 μg pCMV-Pol/Gag, 5 μg pCMV-Rev, and 5 μg pCMV-VSVG) into HEK 293T cells with 90-95% confluency per 10 cm dish. Viral supernatant was harvested 48 hours post-transfection, concentrated by ultracentrifugation, and stored in −80° C. until use. Viral titers were determined by transduction of HT1080 cells and analyzed for EGFP expression with FACS analysis.

Cell Culture and Vector Transduction

HEK 293T and HT1080 cells were purchased from ATCC (Manassas, Va., USA) and maintained in high glucose (4.5 g/1) DMEM supplemented with 2 mM glutamine and 10% FBS. The human CEM T-lymphocytes was cultured in RPMI 1640 medium supplemented with 10% FBS. In embodiments, CEM T lymphocytes were transduced with lentiviral vectors at MOI of 0.5 and 2.5 in the presence of 4 μg/ml polybrene (EMD Millipore, Billerica, Mass.) enhanced by centrifugation. Cells with around 30% EGFP expression were expanded and sorted to purity for further experiments.

CEM T-lymphocytes were transduced with lentiviral vectors as previously described (Li, M. J., and Rossi, J. J., Methods Enzymol., 392, 218-226 (2005b)), with the exception of the multiplicity of infection (MOI) utilized. At an MOI of 50, almost 100% of the cells are EGFP+ as determined by flow cytometry and were used for subsequent experiments without sorting. U373-MAGI-CCR5E cells were obtained through the NIH AIDS Reagent Program (Vodicka, M A, et al. (1997). Indicator cell lines for detection of primary strains of human and simian immunodeficiency viruses. Virology 233: 193-198) and maintained in complete DMEM, supplemented with 0.2 mg/ml G418, 0.1 mg/ml hygromycin B, and 1.0 μg/ml puromycin. Adult CD34+ HSPCs were isolated from G-CSF mobilized peripheral blood (purchased from Progenitor Cell Therapy, Mountain View, Calif.) from health donors under consent and in accordance to institutional IRB approval using previously described methods (Tran, et al. (2012). Optimized processing of growth factor mobilized peripheral blood CD34+ products by counterflow centrifugal elutriation. Stem cells translational medicine 1: 422-429). Briefly, the washed concentrated mobilized peripheral blood were labeled with CliniMACS CD34 microbead (Miltenyi Biotec, Auburn, Calif.) and selected with the CliniMACS Cell Separation System (Miltenyi Biotec, Auburn, Calif.). The purity of the selected CD34 cells was above 95% by FACS analysis. Enriched CD34+ cells were cryopreserved using a controlled rate freezer and stored in liquid nitrogen until use.

For transduced HSPCs used for in vitro CFU assay setup and macrophage differentiation in HIV challenge, HSPCs were thawed and pre-stimulated in StemSpan-SFT6 media [StemSpan (Stem Cell Technologies, Vancouver, British Columbia, Canada) supplemented with 100 ng/ml SCF (Gibco, Grand Island, N.Y.), 100 ng/ml Flt3-L (CellGenix, Freiburg, Germany), 10 ng/ml TPO (CellGenix, Freiburg, Germany), 50 ng/ml IL6 (Life Technologies, Carlsbad, Calif.)], for 48 hours prior to transduction. Lentiviral vectors adjusted to MOI of 20 were added to 6.4×10⁴ pre-stimulated HSPCs in 250 μl StemSpan-SFT6 media in the presence of 20 μg/ml rapamycin (Sigm-Aldrich, St. Louis, Mo.) on RetroNectin (Takara, Mountain View, Calif.)-coated 96-well plate. After 24 hours incubation, the lentiviral vector and rapamycin were washed away and HSPCs were cultured in 1.3 ml StemSpan-SFT6 media supplemented with 0.75 μM SR1 (Cellagen Technologies, San Diego, Calif.) for 5 days prior to sorting. HSPCs were sorted on CD34 marker, in addition to EGFP expression for the transduced population. Only CD34+ (untransduced) or CD34+/GFP+ (transduced) cells were used for subsequent colony forming assay set up and macrophage differentiation for HIV challenge experiments.

For transduced HSPCs used for mice transplantation, HSPCs were pre-stimulated overnight in StemSpan-SFT6 media then transduced with FGLV at MOI=10 at 1×10⁶ cell/ml on Retronectin (Takara, Otsu-Shiga Japan)-coated non-tissue culture T-75 flask (5 μg protein/cm², 1.3-2.6×10⁵ vaiable cells/cm²) for 24 hours in the presence of 20 μg/mL rapamycin. Lentivirus and rapamycin were removed and cells were washed once before being resuspended for transplantation in injection saline solution (APP Pharmaceuticals, Lake Zurich, Ill.).

Generation of Adult CD34+ HSPC Derived Macrophages

Sorted HSPCs were cultured in Iscove's modified Dulbeco's media (IMDM) with 20% FBS supplemented with 2 mM glutamine, 25 ng/ml SCF (Gibco, Grand Island, N.Y.), 30 ng/ml Flt-3L (CellGenix, Freiburg, Germany), 30 ng/ml IL3 (Gibco, Grand Island, N.Y.), 30 ng/ml M-CSF (PeptroTech, Rocky Hill, N.J.) for 10 days for guided differentiation to monoctyes, then switched to DMEM with 10% FBS supplemented with 2 mM glutamine, 10 ng/ml GM-CSF (Leukine, Sanofi U S, Bridgewater, N.J.), 10 ng/ml M-CSF (PeptroTech, Rocky Hill, N.J.) for 5 days for activation into macrophages. Adherent macrophage cells were collected for HIV challenge experiments. The purity of cells was typically greater than 90% CD14+ based on FACS analysis.

Flow Cytometric Assay for CCR5 Knockdown Studies in U373-MAGI-CCR5E Cells

About 1×10⁵ U373-MAGI-CCR5E cells were seeded per well in a 24-well dish one day prior to transfection. An equal ratio of pHIV7-EGFP to tRNA^{Ser} construct in pBluescript with 400 ng total plasmid DNA were transiently transfected with Lipofectamine 2000 (Life Technologies, Carlsbed, Calif.) following manufacturer's instructions. The EGFP marker from the co-transfected pHIV7-EGFP plasmid serves as an internal control for transfection efficiency. Cells were detached 72 hours later and stained with CCR5-APC antibody (clone 2D7, BD Pharmingen, San Jose, Calif.) to estimate CCR5 knockdown by flow cytometry. Data were collected on Gallios flow cytometer (Beckman Coulter, Brea, Calif.) and analyzed by FCS express version 4 software (De Novo software, Los Angeles, Calif.).

Northern Blotting Analysis

Approximately 2 μg of pcDNA3-U1-S1/S2M/S3B-Ult with tRNA^{Ser}-CCR5sh cassette in either orientation was transfected into HEK 293 cells with 90-95% confluency per well in E-well dish with Lipofectamine 2000 with manufacturer's protocol. Total RNA was extracted 48 hours later with STAT-60 according to manufacturer's protocol. For each construct, 10 μg of total RNA were loaded onto 8% polyacrylamide gel with 8M urea then electroblotted onto a Hybond-N nylon membrane (Amersham, Arlington, Height, Ill.) and hybridized with P³²-labeled DNA probe specific for small RNA. Small nuclear U2A RNA serves as internal control. Total RNA from sorted CEM T lymphocytes were isolated similarly and Northern blotting analysis was performed as described above, with the exception of 20 μg of total RNA used as input. The U6 small nuclear RNA was used as a loading control.

The small RNA probe sequences are given below:

S1:
(SEQ ID NO: 17)
5′-GCG GAG ACA GCG ACG AAG AGC-3′
S2M:
(SEQ ID NO: 18)
5′-GCC TGT GCC TCT TCA GCT ACC-3′
S3B:
(SEQ ID NO: 19)
5′-CAT CTC CTA TGG CAG GAA GAA-3′
U16RBE:
(SEQ ID NO: 20)
5′-CGT CAG CGT CAT TGA CGC TGC GCC CA-3′
U16U5RZ (U5RZ):
(SEQ ID NO: 21)
5′-GAG TGC TTT TCG AAA ACT CAT CAG AA-3′
U16TAR:
(SEQ ID NO: 22)
5′-CCA GAG AGC TCC CAG GCT CAG-3′
U6:
(SEQ ID NO: 23)
5′-TAT GGA ACG CTT CTC GAA TT-3′
CCR5sh:
(SEQ ID NO: 24)
5′-AAA GTG TCA AGT CCA ATC TAT GA-3′
CCR5RZ:
(SEQ ID NO: 25)
5′ GTG TCA AGT TTC GTC CAC ACG GAC TCA TCA
GCA ATC TA-3′
U2A:
(SEQ ID NO: 26)
5′-AGA ACA GAT ACT ACA CTT GA-3′

HIV-1 Challenge, p24 Antigen Assays and Intracellular HIV Staining for Monitor Viral Replica

One million untransduced or stably transduced CEM-T lymphocytes were infected in triplicate with the NL4-3 strain of HIV-1 at an MOI of 0.01. After overnight incubation, cells were washed three times with Hank's balanced salts solution and cultured in RPMI 1640 with 10% FBS. At designated time points, culture supernatants were collected and analyzed for HIV-1 replication by a p24 ELISA assay (Perkin Elmer, USA) according to the manufacturer's protocol.

About 1×10⁵ CD34-derived macrophages were infected in triplicate with HIV-1 Bal strain at MOI of 0.01. After overnight incubation, the HIV virus was removed and the cells were cultured in DMEM with 10% FBS supplemented with 2 mM glutamine, 10 ng/ml GM-CSF, 10 ng/ml M-CSF.

Viral replication was analyzed by intracellular staining of HIV core proteins at indicated time points. Cells were detached with Accutase solution (Sigma-Aldrich, St. Louis, Mo.), viability estimated by LIVE/DEAD fixable aqua dead cell stain (Sigma-Aldrich, St. Louis, Mo.), fixed and permeabilized with intracellular fixation and permeabilization buffer set (eBioscience, San Diego, Calif.) before staining with KC57-RD antibody (KC57-RD1 antibody, clone FH190-1-1, Beckman Coulter, Brea, Calif.) that recognizes HIV-1 core proteins and finally analyzed by flow cytometry. Data were collected on Gallios flow cytometer and analyzed by FCS express version 4 software.

Real-Time Quantitative RT-PCR to Quantify Anti-HIV RNA Expression

Total RNA from stably transduced CEM T-lymphocytes challenged with HIV-1 was extracted with STAT-60 reagent (Tel-Test, Friendswood, Tex., USA) according to the manufacturer's instructions then resuspended in nuclease-free water. Residual DNA was digested using Turbo DNase (Ambion, USA) with 1 ng of total RNA in a 10 n1 reaction following the manufacturer's instructions. Both 51 siRNA and U16TAR RNA decoy expression were analyzed by real time qRT-PCR with the CFX96 Real-Time Detection System (Bio-Rad, Hercules, Calif., USA) and expression levels were normalized to the U6 small nuclear RNA. 51 siRNA was reverse transcribed into cDNA using the TaqMan MicroRNA Reverse Transcription kit (Applied Biosystems, Foster City, Calif., USA) with 100 ng DNase-treated total RNA and stem-loop RT primer according to the manufacturer's instruction. Real time PCR was carried out with 1.3 n1 of RT reaction, 0.2 μM S1-specific probe, 1.5 μM forward primer, 0.7 μM reverse primer in TaqMan Universal PCR Master Mix diluted to 1× concentration (Applied Biosystems, Foster City, Calif., USA) in a final volume of 20 μl. PCR conditions were 95° C. for 10 min, followed by 40 cycles of 95° C. for 30 s, 64° C. for 30 s, 72° C. for 30 s (DiGiusto, D. L. et al., Sci Transl Med., 2, 36-43 (2010)). The exact copy number of 51 siRNA was determined using a standard curve constructed with known concentrations of synthetic 51 RNA oligo (Integrated DNA Technology).

The U16TAR RNA decoy and the internal control small nuclear U6 RNA were reverse transcribed using 200 ng of DNase-treated total RNA with 50 ng of random primers (Invitrogen, USA) and Moloney Murine Leukemia Virus Reverse Transcriptase (Invitrogen, USA) in a 20 μl reaction according to the manufacturer's instructions. Real time PCR for the U16TAR RNA decoy was carried out with 1 μl of the RT reaction, 0.2 μM TAR-specific probe, 0.5 μM of each U16-specific forward and reverse primers in TaqMan Universal PCR Master Mix diluted to 1× concentrations (Applied Biosystems, Foster City, Calif., USA) in a final volume of 20 μl. The PCR conditions were 95° C. for 10 min, followed by 40 cycles of 95° C. for 30 s, and 64° C. for 1 min. The exact copy of RNA molecules were determined with a standard curve constructed with known concentrations of U16TAR plasmid. Quantification of the U6 internal control was accomplished using 2 μl of the RT reaction with 0.4 μM of each U6-specific forward and reverse primers utilizing iQ SYBR Green Supermix (Bio-Rad, Hercules, Calif., USA) in a final volume of 25 μl. The PCR conditions were 95° C. for 5 min, followed by 40 cycles of 95° C. for 30 s, 60° C. for 30 s, and 72° C. for 30 s. A standard curve with known amounts of total RNA input was utilized to determine the precise RNA input to account for sample-to-sample variability.

Quantitative RT-PCR primer sequences are given below:

S1:
Stem-loop RT primer:
(SEQ ID NO: 27)
5′-GTC GTA TCC AGT GCA GGG TCC GAG GTA TTC
GCA CTG GAT ACG ACA GCG GA-3′
Probe:
(SEQ ID NO: 28))
5′-(6-FAM)-TCG CAC TGG ATA CGA CAG CGG AGA
CA-(BHQ1)-3′
Forward:
(SEQ ID NO: 29)
5′-GCC TCT TCG TCG CTG TCT-3′
Reverse:
(SEQ ID NO: 30)
5′-GTG CAG GGT CCG AGG T-3′
U16TAR:
Probe:
(SEQ ID NO: 31)
5′-(6-FAM)-ATC TGA GCC TGG GAG CTC TCT GGC
T-(BHQ1)-3′
Forward:
(SEQ ID NO: 32)
5′-TGC GTC TTA CTC TGT TCT CAG CGA-3′
Reverse:
(SEQ ID NO: 33)
5′-CGT CAA CCT TCT GTA CCA GCT TAC-3′
U6:
Forward:
(SEQ ID NO: 34)
5′-GCT CGC TTC GGC AGC ACA TAT ACT AA-3′
Reverse:
(SEQ ID NO: 35)
5′-ACG AAT TTG CGT GTC ATC CTT GCG-3′

Real-Time Quantitative RT-PCR for CCR5 Knockdown Studies in Macrophages Differentiated from Adult HSPCs

Total RNA from CD34-derived macrophages were extracted with STAT-60 reagent (Tel-Test, Friendswood, Tex.) with manufacturer's protocol then resuspended in nuclease-free water. Residual DNA was digested with Ambion TURBO DNase (Life Technologies, Carlsbed, Calif.) with 2 μg of total RNA in a 15-μl reaction, in accordance with manufacturer's instructions. Complementary DNA was then synthesized with 1 μg of DNase-treated RNA with 100 ng of random primers (Invitrogen, Carlsbad, Calif.) and Moloney murine leukemia virus reverse transcriptase (Invitrogen, Carlsbad, Calif.) in a 27-μl reaction according to manufacturer's instructions. Real time PCR was carried out with CFX96 real-time detection system with 10 ng of cDNA, 0.4 μM of each gene specific (CCR5 or GAPDH) primers with iQ SYBR green supermix (Bio-Rad, Hercules, Calif.) in a final volume of 25 μl. The PCR conditions were 95° C. for 10 min, followed by 40 cycles of 95° C. for 20 s, 62° C. for 1 min. A standard curve with known serial dilutions of total RNA input was utilized to calculate the ratio between CCR5 and GAPDH to estimate percentage of CCR5 down-regulation.

Primer sequences for PCR were as follows:

| (SEQ ID NO: 36) | |
|---|---|
| CCR5-F: | 5′-TTC ATT ACA CCT GCA GCT CTC-3′; |
| (SEQ ID NO: 37) | |
| CCR5-R: | 5′-CCT GTT AGA GCT ACT GCA ATT AT-3′; |
| (SEQ ID NO: 38) | |
| GAPDH-F: | 5′-CGC TCT CTG CTC CTC CTG TT-3′; |
| (SEQ ID NO: 39) | |
| GAPDH-R: | 5′-CCA TGG TGT CTG AGC GAT GT-3′. |

In Vitro CFU Assay for Adult CD34+ HSPCs

A total of 500 sorted CD34+ cells were plated in triplicate in MethoCult H4435-enriched methylcellulose media (Stem Cell Technologies, Vancouver, British Columbia, Canada) according to manufacturer's protocol. Cells were cultured for 12 to 13 days before colony scoring under inverted microscope.

Fc/IL-7 Protein Production

Fc/IL-7 was cloned into an OptiVect-TOPO (Invitrogen, Carlsbad, Calif.) vector and protein was produced from a cloned transfected DG44 CHO cell line as per the methods of Lo et al ((1998). High level expression and secretion of Fc-X fusion proteins in mammalian cells. Protein engineering 11: 495-500).

Humanized NSG Mouse Model

NOD.Cg-Prkdc^{scid} IL2rg^{tmlWjl}/SzJ (NSG) mice were obtained from The Jackson Laboratory (Bar Harbor, Me.) and bred at the City of Hope Animal Resources Center according to protocols approved by the Institutional Animal Care and Use Committee of the City of Hope. Adult (8-10 week old) NSG mice were irradiated at 270 cGy 24 hours prior to transplantation. Each animal was transplanted with 1×10⁶HSPCs following transduction as described via intravenous injection. To enhance T lymphopoiesis, 20 μg Fc/IL7 protein was administered per animal intravenously weekly for 11 weeks.

In Vivo O⁶-BG and BCNU Selection for Enrichment of Gene Modified Cells

O⁶-BG (Sigma-Aldrich, St. Louis, Mo.) was prepared in 4% DMSO, 30% PEG-400 (Sigma-Aldrich, St. Louis, Mo.) and 66% injection saline solution. BCNU (injectable carmustine) was purchased from Bristol-Myers Squibb and stock solution was reconstituted in supplied absolute ethanol at 100 mg/3 ml, as per manufacturer's instructions, then diluted in injection saline solution before administration. Drug selection was performed at either at the 7^{th} and 8^{th} weeks (2× treatment cohort, N=12 mice), or 7^{th}, 8^{th}, and 9^{th} weeks (3× treatment cohort, N=12 mice) post-transplantation. Control cohort (N=12 mice) received saline injection. Animals in treatment cohorts received 20 mg/kg O⁶-BG followed by 5 mg/kg BCNU 1.5 hour later via IP injection for each round of drug selection.

Flow Cytometric Analysis of Engraftment and Gene Modification Frequency

Mice were necropsied 11 weeks after transplantation for analysis of engraftment and enrichment of gene modified cells. Single cell suspensions of bone marrow (femurs) and spleen were prepared by mechanical dissociation and red cells lysed using ACK lysis Buffer (Sigma-Aldrich, St. Louis, Mo.). All cell suspensions were pre-treated with human immunoglobulin (GammaGard, Baxter Healthcare Corp. Deerfield, Ill.) for 30 minutes to block nonspecific antibody staining. Spleenocytes were stained with a human pan-leukocyte antibody to CD45-PC5 (BioLegend, San Diego, Calif.), and lineage specific anti-human antibodies, CD3-ECD, CD4-APC, and CD14-APC-Alexa-750 (Invitrogen, Carlsbad, Calif.) for 20 minutes and washed 2 times with 1 mL of PBS containing 0.1% BSA (Sigma-Aldrich, St Louis, Mo.). Bone marrow cells were stained with antibodies to human CD45-PC5 (Beckman Coulter, Brea, Calif.), for 20 minutes and washed 2 times with 1 mL of PBS containing 0.1% BSA. To establish analytical gates and background staining, bone marrow and spleen samples from two to three untransplanted mice were stained with the same antibody panel. Data were collected on Gallios flow cytometer and analyzed by FCS express version 4 software.

Statistical Analyses

The average and standard deviation for S1 siRNA and U16TAR RNA decoy expression were derived from three independent measurements. Data were analyzed by the statistical software Prism version 5.01 (GraphPad Software, La Jolla, Calif., USA) using one-way ANOVA followed by Bonferroni's multiple comparsion test. P-values less than or equal to 0.05 were considered as statistically significant compared to cells cultured under identical conditions in the absence of HIV (i.e., D0).

In vitro CFU data were analyzed with statistical software Prism version 6.01 (GraphPad Software, La Jolla, Calif.), using one-way ANOVA followed by Bonferroni's comparison test. Values of p less than or equal to 0.05 were considered statically significant compared to untransduced control. The average and standard deviation were derived from two to three independent donors as indicated. In vivo drug selection data were also analyzed with Prism software using two-way ANOVA followed by two-tailed t-test. Values of p less than or equal to 0.05 were considered statistically significant compared to control animals. The average and standard deviation were derived from cohorts of 12 animals per group.

### Tables

TABLE 1
Packaging efficiencies of lentiviral vectors with MCM7 transgene. ^{a}Viral titer
is determined by transducing HT1080 cells with unconcentrated viral supernatant and reported in
transduction units per milliliter (TU/ml). Samples with ~30-40% EGFP+ cells, determined from
flow cytometry, were used for calculation. The values are averages of two independent
experiments.

| | Viral Titer | | | | |
|---|---|---|---|---|---|
| | (TU/ml)^{a} | | | | |
| | (1.41 ± 0.66) × | Ratio | | Ratio | Ratio |
| | 10⁶ | (to | | (to | (Forward |
| Construct | Forward- | pHIV7- | Reverse- | pHIV7- | to |
| pHIV7-EGFP (empty) | U1t | EGFP) | U1t | EGFP) | Reverse) |
| MCM7-S1/S2M/S3B | (4.01 ± 1.23) × | 2.84 | (4.20 ± 0.50) × | 0.030 | 95.5 |
| | 10⁶ | | 10⁴ | | |
| MCM7-S1/S2M/U16TAR | (4.11 ± 1.24) × | 2.91 | (3.00 ± 0.42) × | 0.021 | 137 |
| | 10⁶ | | 10⁴ | | |
| MCM7-S1/U16U5RZ/U16TAR | (4.34 ± 1.46) × | 3.08 | (1.44 ± 0.34) × | 0.010 | 301 |
| | 10⁶ | | 10⁴ | | |
| MCM7-U16RBE/S2M/U16TAR | (4.66 ± 1.72) × | 3.30 | (2.41 ± 0.45) × | 0.017 | 193 |
| | 10⁶ | | 10⁴ | | |
| MCM7- | (4.12 ± 1.58) × | 2.92 | (1.18 ± 0.22) × | 0.008 | 349 |
| U16RBE/U16U5RZ/U16TAR | 10⁶ | | 10⁴ | | |

TABLE 2
Examples of si-/snoRNA combinations constructed and analyzed.

| Constructs | RNA1 | RNA2 | RNA3 |
|---|---|---|---|
| S1/S2M/S3B | S1 | S2M | S3B |
| S1/S2M/U16TAR | S1 | S2M | U16TAR |
| S1/U5RZ/U16TAR | S1 | U16U5RZ | U16TAR |
| RBE/S2M/U16TAR | U16RBE | S2M | U16TAR |
| RBE/U16U5RZ/U16TAR | U16RBE | U16U5RZ | U16TAR |

TABLE 3
Analysis of combinations of promoter/termination sequences and
transgene orientations.

| | | Reverse U1t |
|---|---|---|
| Unconcentrated viral titer | Forward-U1t | Viral titer |
| Construct | Viral titer (TU/ml) | (TU/ml) |
| MCM7-S1/S2M/S3B | 4.01 ± 1.23 × 10⁶ | 4.20 ± 0.50 × 10⁴ |
| MCM7-S1/S2M/U16TAR | 4.11 ± 1.24 × 10⁶ | 3.00 ± 0.42 × 10⁴ |
| MCM7-S1/U16U5RZ/U16TAR | 4.34 ± 1.46 × 10⁶ | 1.44 ± 0.34 × 10⁴ |
| MCM7-U16RBE/S2M/U16TAR | 4.66 ± 1.72 × 10⁶ | 2.41 ± 0.45 × 10⁴ |
| MCM7-U16RBE/U16U5RZ/ | 4.12 ± 1.58 × 10⁶ | 4.18 ± 0.22 × 10⁴ |
| U16TAR | | |

TABLE 4
Analysis of combinations of promoter/termination sequences and transgene
orientations.

| | Concentrated Viral | |
|---|---|---|
| | Titer (TU/ml) | |
| | Forward (with U1 | Reverse (with |
| Construct | terminator)^{b} | BGHpA)^{c} |
| MCM7-S1/S2M/S3B | 8.01 × 10⁸ | 5.70 × 10⁷ |
| MCM7-S1/S2M/TAR | 8.61 × 10⁸ | 5.88 × 10⁷ |
| MCM7-S1/U16U5RZ/U16TAR | 6.31 × 10⁸ | 7.96 × 10⁶ |
| MCM7-U16RBE/S2M/U16TAR | 6.75 × 10⁸ | 3.14 × 10⁷ |
| MCM7-U16RBE/U16U5RZ/ | 5.08 × 10⁸ | 1.39 × 10⁷ |
| U16TAR | | |

TABLE 5
Examples of promoter and termination sequences.

| RNA promoter 2 | RNA4 | Termination seq 2 | orientation |
|---|---|---|---|
| tRNA^{Ser} | Bifunctional | Pol III term seq | Forward |
| | siRNA as shRNA | | |
| | (against CCR5 | | |
| | and HIV UTR) | | |
| tRNA^{Ser} | Bifunctional | Pol III term seq | Reverse |
| | siRNA as shRNA | | |
| | (against CCR5 | | |
| | and HIV UTR) | | |
| tRNA^{Ser} | Bifunctional | Pol III term seq | Forward |
| | siRNA as pre- | | |
| | miRNA (against | | |
| | CCR5 and HIV | | |
| | UTR) | | |
| tRNA^{Ser} | Bifunctional | Pol III term seq | Reverse |
| | siRNA as pre- | | |
| | miRNA (against | | |
| | CCR5 and HIV | | |
| | UTR) | | |
| tRNA^{Ser} | siRNA as shRNA | Pol III term seq | Forward |
| | (CCR5-sh12) | | |
| tRNA^{Ser} | siRNA as shRNA | Pol III term seq | Reverse |
| | (CCR5-sh12) | | |

TABLE 6
Unconcentrated Viral titer of various MCM7 constructs (TU/ml):

| | MCM7-S1/S2M/S3B- | MCM7-S1/S2M/S3B- |
|---|---|---|
| MCM7-S1/S2M/S3B | F12sh | R12sh |
| 2.41e6 | 1.31e6 | 1.75e6 |
| | MCM7- | |
| MCM7-S1/ | S1/S2M/U16TAR- | MCM7-S1/S2M/ |
| S2M/U16TAR | F12sh | U16TAR-R12sh |
| 2.36e6 | 1.78e6 | 2.46e6 |
| | | MCM7- |
| MCM7- | MCM7-S1/U16U5RZ/ | S1/U16U5RZ/ |
| S1/U16U5RZ/U16TAR | U16TAR-F12sh | U16TAR-R12sh |
| 2.65e6 | 1.77e6 | 1.91e6 |

TABLE 7
Lentiviral Vector Constructs used in this study. In each column,
sh indicates short hairpin RNA, decoy indicates RNA decoy,
Rx indicates ribozyme, tat is HIV tat RNA, rev is HIV rev RNA,
tat/rev is the shared sequence between HIV rev and tat RNAs,
CCR5 is the cellular co-receptor for R5 tropic HIV.

| Vector | RNA 1 | RNA 2 | RNA 3 | RNA 4 |
|---|---|---|---|---|
| FGLV | sh-tat/rev | TAR decoy | CCR5 Rz | — |
| SGLV2 | sh-tat/rev | sh-rev | sh-tat | — |
| SGLV4 | sh-tat/rev | sh-rev | sh-tat | sh-CCR5 |

### EMBODIMENTS

### Embodiment 1

A recombinant nucleic acid encoding an antiviral polycistronic RNA, said recombinant nucleic acid comprising a first RNA promoter operably linked to: (i) a first antiviral RNA encoding sequence, (ii) a second antiviral RNA encoding sequence and a (iii) third antiviral RNA encoding sequence, wherein said first RNA promoter is a forward promoter.

### Embodiment 2

The recombinant nucleic acid of claim 1, further comprising a second RNA promoter operably linked to a viral entry inhibiting RNA encoding sequence, wherein said second RNA promoter is a reverse promoter.

### Embodiment 3

The recombinant nucleic acid of claim 2, wherein said recombinant nucleic acid forms part of a viral expression vector.

### Embodiment 4

The recombinant nucleic acid of claim 1 or 2, wherein said recombinant nucleic acid forms part of a recombinant viral particle.

### Embodiment 5

The recombinant nucleic acid of any one of claims 1-3, wherein said first RNA promoter is a RNA polymerase II promoter.

### Embodiment 6

The recombinant nucleic acid of claim 5, wherein said RNA polymerase II promoter is a small nuclear RNA (snRNA) promoter.

### Embodiment 7

The recombinant nucleic acid of claim 6, wherein said snRNA promoter is a U1 promoter.

### Embodiment 8

The recombinant nucleic acid of any one of claims 2-7, wherein said first antiviral RNA encoding sequence encodes a first small interfering RNA (siRNA), said second antiviral RNA encoding sequence encodes a second siRNA and said third antiviral RNA encoding sequence encodes a third siRNA.

### Embodiment 9

The recombinant nucleic acid of claim 8, wherein said first siRNA, second siRNA and third siRNA are independently a viral transcription inhibiting siRNA, a viral replication inhibiting siRNA, a viral transcription and replication inhibiting siRNA, a ribozyme or an RNA decoy.

### Embodiment 10

The recombinant nucleic acid of claim 9, wherein said viral transcription inhibiting siRNA is a Tat siRNA.

### Embodiment 11

The recombinant nucleic acid of claim 9 or 10, wherein said viral replication inhibiting siRNA is a Rev siRNA.

### Embodiment 12

The recombinant nucleic acid of any one of claims 9-11, wherein said viral transcription and replication inhibiting siRNA is a Tat/Rev siRNA.

### Embodiment 13

The recombinant nucleic acid of any one of claims 9-12, wherein said ribozyme is a small nucleolar (sno) RNA.

### Embodiment 14

The recombinant nucleic acid of claim 13, wherein said snoRNA is a U5 ribozyme.

### Embodiment 15

The recombinant nucleic acid of any one of claims 9-14, wherein said RNA decoy is a snoRNA.

### Embodiment 16

The recombinant nucleic acid of claim 15, wherein said snoRNA is a rev binding RNA decoy or a Tat binding RNA decoy.

### Embodiment 17

The recombinant nucleic acid of any one of claims 2-16, wherein said second RNA promoter is downstream of said third antiviral RNA encoding sequence.

### Embodiment 18

The recombinant nucleic acid of any one of claims 2-16, wherein said second RNA promoter is a polymerase III promoter.

### Embodiment 19

The recombinant nucleic acid of claim 18, wherein said RNA polymerase III promoter is a small nuclear RNA (snRNA) promoter.

### Embodiment 20

The recombinant nucleic acid of claim 19, wherein said snRNA promoter is a U6 promoter.

### Embodiment 21

The recombinant nucleic acid of any one of claims 2-20, wherein said viral entry inhibiting RNA encoding sequence encodes a cellular receptor siRNA.

### Embodiment 22

The recombinant nucleic acid of claim 21, wherein said cellular receptor siRNA is a T cell receptor siRNA.

### Embodiment 23

The recombinant nucleic acid of claim 22, wherein said T cell receptor siRNA is a small hairpin (sh) RNA.

### Embodiment 24

The recombinant nucleic acid of claim 23, wherein said shRNA is a CCR5 shRNA.

### Embodiment 25

The recombinant nucleic acid of claim 23, wherein said shRNA is a CXCR4 shRNA.

### Embodiment 26

The recombinant nucleic acid of any one of claims 2-25, wherein said viral entry inhibiting RNA encoding sequence encodes a nuclear receptor siRNA.

### Embodiment 27

The recombinant nucleic acid of claim 26, wherein said nuclear receptor siRNA is a transportin 3 (TNPO3) siRNA.

### Embodiment 28

The recombinant nucleic acid of any one of claims 1-27, further comprising a transcriptional terminator sequence.

### Embodiment 29

The recombinant nucleic acid of any one of claims 2-28, further comprising a transcriptional terminator sequence.

### Embodiment 30

The recombinant nucleic acid of claim 29, wherein said transcriptional terminator sequence is an U1 terminator sequence.

### Embodiment 31

The recombinant nucleic acid of claim 29, wherein said transcriptional terminator sequence is downstream of said viral entry inhibiting RNA encoding sequence.

### Embodiment 32

The recombinant nucleic acid of any one of claims 1-31, further comprising a first nucleic acid linker connecting said first antiviral RNA encoding sequence to said second antiviral RNA encoding sequence and a second nucleic acid linker connecting said second antiviral RNA encoding sequence to said third antiviral RNA encoding sequence.

### Embodiment 33

The recombinant nucleic acid of claim 32, wherein said first nucleic acid linker or said second nucleic acid linker comprise an intron sequence.

### Embodiment 34

The recombinant nucleic acid of claim 32, wherein said intron sequence is a MCMI intron sequence.

### Embodiment 35

The recombinant nucleic acid of any one of claims 2-34, further comprising an antiviral protein encoding sequence.

### Embodiment 36

The recombinant nucleic acid of claim 35, wherein said antiviral protein encoding sequence is downstream of said viral entry inhibiting RNA encoding sequence.

### Embodiment 37

The recombinant nucleic acid of claim 35, wherein said antiviral protein encoding sequence encodes a C46 fusion inhibitor.

### Embodiment 38

The recombinant nucleic acid of claim 35, wherein said antiviral protein encoding sequence encodes a mutant Rev protein.

### Embodiment 39

The recombinant nucleic acid of claim 38, wherein said mutant Rev protein is a Rev M10 protein.

### Embodiment 40

The recombinant nucleic acid of claim 35, further comprising a transcriptional terminator sequence.

### Embodiment 41

The recombinant nucleic acid of claim 40, wherein said transcriptional terminator sequence is an U1 terminator sequence.

### Embodiment 42

The recombinant nucleic acid of claim 40 or 41, wherein said transcriptional terminator sequence is downstream of said antiviral protein encoding sequence.

### Embodiment 43

The recombinant nucleic acid of any one of claims 2-42, wherein said first RNA promoter is a U1 promoter, said first antiviral RNA encoding sequence encodes a Tat/Rev siRNA, said second antiviral RNA encoding sequence encodes a Rev siRNA, said third antiviral RNA encoding sequence encodes a Tat siRNA, said second RNA promoter is a U6 promoter, and said viral entry inhibiting RNA encoding sequence encodes a CCR5 shRNA.

### Embodiment 44

The recombinant nucleic of any one of claims 2-42, wherein said first RNA promoter is a U1 promoter, said first antiviral RNA encoding sequence encodes a Tat/Rev siRNA, said second antiviral RNA encoding sequence encodes a Rev siRNA, said third antiviral RNA encoding sequence encodes a Tat binding RNA decoy, said second RNA promoter is a U6 promoter, and said viral entry inhibiting RNA encoding sequence encodes a CCR5 shRNA.

### Embodiment 45

The recombinant nucleic acid of any one of claims 2-42, wherein said first RNA promoter is a U1 promoter, said first antiviral RNA encoding sequence encodes a Tat/Rev siRNA, said second antiviral RNA encoding sequence encodes a U5 ribozyme, said third antiviral RNA encoding sequence encodes a Tat binding RNA decoy, said second RNA promoter is a U6 promoter, and said viral entry inhibiting RNA encoding sequence encodes a CCR5 shRNA.

### Embodiment 46

A recombinant nucleic acid encoding an antiviral polycistronic RNA, said recombinant nucleic acid comprising: (i) a first RNA promoter operably linked to: a first antiviral RNA encoding sequence, a second antiviral RNA encoding sequence and a third antiviral RNA encoding sequence; and (ii) a second RNA promoter operably linked to a viral entry inhibiting RNA encoding sequence.

### Embodiment 47

The recombinant nucleic acid of claim 46, wherein said recombinant nucleic acid forms part of a viral expression vector.

### Embodiment 48

The recombinant nucleic acid of claim 46 or 47, wherein said recombinant nucleic acid forms part of a recombinant viral particle.

### Embodiment 49

The recombinant nucleic acid of any one of claims 46-48, wherein said first RNA promoter is a RNA polymerase II promoter.

### Embodiment 50

The recombinant nucleic acid of claim 49, wherein said RNA polymerase II promoter is a small nuclear RNA (snRNA) promoter.

### Embodiment 51

The recombinant nucleic acid of claim 50, wherein said snRNA promoter is a U1 promoter.

### Embodiment 52

The recombinant nucleic acid of any one of claims 46-51, wherein said first antiviral RNA encoding sequence encodes a first small interfering RNA (siRNA), said second antiviral RNA encoding sequence encodes a second siRNA and said third antiviral RNA encoding sequence encodes a third siRNA.

### Embodiment 53

The recombinant nucleic acid of claim 52, wherein said first siRNA, second siRNA and third siRNA are independently a viral transcription inhibiting siRNA, a viral replication inhibiting siRNA, a viral transcription and replication inhibiting siRNA, a ribozyme or an RNA decoy.

### Embodiment 54

The recombinant nucleic acid of claim 53, wherein said viral transcription inhibiting siRNA is a Tat siRNA.

### Embodiment 55

The recombinant nucleic acid of claim 53, wherein said viral replication inhibiting siRNA is a Rev siRNA.

### Embodiment 56

The recombinant nucleic acid of claim 53, wherein said viral transcription and replication inhibiting siRNA is a Tat/Rev siRNA.

### Embodiment 57

The recombinant nucleic acid of claim 53, wherein said ribozyme is a small nucleolar (sno) RNA.

### Embodiment 58

The recombinant nucleic acid of claim 57, wherein said snoRNA is a U5 ribozyme.

### Embodiment 59

The recombinant nucleic acid of claim 53, wherein said RNA decoy is a snoRNA.

### Embodiment 60

The recombinant nucleic acid of claim 59, wherein said snoRNA is a rev binding RNA decoy or a Tat binding RNA decoy.

### Embodiment 61

The recombinant nucleic acid of any one of claims 46-60, wherein said second RNA promoter is downstream of said third antiviral RNA encoding sequence.

### Embodiment 62

The recombinant nucleic acid of any one of claims 46-61, wherein said second RNA promoter is a polymerase III promoter.

### Embodiment 63

The recombinant nucleic acid of claim 62, wherein said RNA polymerase III promoter is a small nuclear RNA (snRNA) promoter.

### Embodiment 64

The recombinant nucleic acid of claim 63, wherein said snRNA promoter is a U6 promoter.

### Embodiment 65

The recombinant nucleic acid of any one of claims 46-64, wherein said viral entry inhibiting RNA encoding sequence encodes a cellular receptor siRNA.

### Embodiment 66

The recombinant nucleic acid of claim 65, wherein said cellular receptor siRNA is a T cell receptor siRNA.

### Embodiment 67

The recombinant nucleic acid of claim 66, wherein said T cell receptor siRNA is a small hairpin (sh) RNA.

### Embodiment 68

The recombinant nucleic acid of claim 67, wherein said shRNA is a CCR5 shRNA.

### Embodiment 69

The recombinant nucleic acid of claim 67, wherein said shRNA is a CXCR4 shRNA.

### Embodiment 70

The recombinant nucleic acid of any one of claims 46-69, wherein said viral entry inhibiting RNA encoding sequence encodes a nuclear receptor siRNA.

### Embodiment 71

The recombinant nucleic acid of claim 70, wherein said nuclear receptor siRNA is a transportin 3 (TNPO3) siRNA.

### Embodiment 72

The recombinant nucleic acid of any one of claims 46-71, further comprising a transcriptional terminator sequence.

### Embodiment 73

The recombinant nucleic acid of claim 72, wherein said transcriptional terminator sequence is an U1 terminator sequence.

### Embodiment 74

The recombinant nucleic acid of claim 72, wherein said transcriptional terminator sequence is downstream of said viral entry inhibiting RNA encoding sequence.

### Embodiment 75

The recombinant nucleic acid of any one of claims 46-74, further comprising a first nucleic acid linker connecting said first antiviral RNA encoding sequence to said second antiviral RNA encoding sequence and a second nucleic acid linker connecting said second antiviral RNA encoding sequence to said third antiviral RNA encoding sequence.

### Embodiment 76

The recombinant nucleic acid of claim 75, wherein said first nucleic acid linker or said second nucleic acid linker comprise an intron sequence.

### Embodiment 77

The recombinant nucleic acid of claim 76, wherein said intron sequence is a MCMI intron sequence.

### Embodiment 78

The recombinant nucleic acid of any one of claims 46-77, wherein said first RNA promoter is a U1 promoter, said first antiviral RNA encoding sequence encodes a Tat/Rev siRNA, said second antiviral RNA encoding sequence encodes a Rev siRNA, said third antiviral RNA encoding sequence encodes a Tat siRNA, said second RNA promoter is a U6 promoter and said viral entry inhibiting RNA encoding sequence encodes a CCR5 shRNA.

### Embodiment 79

The recombinant nucleic acid of any one of claims 46-77, wherein said first RNA promoter is a U1 promoter, said first antiviral RNA encoding sequence encodes a Tat/Rev siRNA, said second antiviral RNA encoding sequence encodes a Rev siRNA, said third antiviral RNA encoding sequence encodes a Tat binding RNA decoy, said second RNA promoter is a U6 promoter, and said viral entry inhibiting RNA encoding sequence encodes a CCR5 shRNA.

### Embodiment 80

The recombinant nucleic acid of any one of claims 46-77, wherein said first RNA promoter is a U1 promoter, said first antiviral RNA encoding sequence encodes a Tat/Rev siRNA, said second antiviral RNA encoding sequence encodes a U5 ribozyme, said third antiviral RNA encoding sequence encodes a Tat binding RNA decoy, said second RNA promoter is a U6 promoter, and said viral entry inhibiting RNA encoding sequence encodes a CCR5 shRNA.

### Embodiment 81

A mammalian cell comprising a recombinant antiviral polycistronic RNA comprising: (i) a first antiviral RNA, a second antiviral RNA and a third antiviral RNA; and (ii) a viral entry inhibiting RNA.

### Embodiment 82

The mammalian cell of claim 81, wherein said first antiviral RNA, said second antiviral RNA and said third antiviral RNA is a small interfering RNA (siRNA).

### Embodiment 83

The mammalian cell of claim 82, wherein said siRNA is a viral transcription inhibiting siRNA, a viral replication inhibiting siRNA, a viral transcription and replication inhibiting siRNA, a ribozyme or an RNA decoy.

### Embodiment 84

The mammalian cell of claim 83, wherein said viral transcription inhibiting siRNA is a Tat siRNA.

### Embodiment 85

The mammalian cell of claim 83 or 84, wherein said viral replication inhibiting siRNA is a Rev siRNA.

### Embodiment 86

The mammalian cell of any one of claim 83, wherein said viral transcription and replication inhibiting siRNA is a Tat/Rev siRNA.

### Embodiment 87

The mammalian cell of any one of claims 83-86, wherein said ribozyme is a snoRNA.

### Embodiment 88

The mammalian cell of claim 87, wherein said ribozyme is a U5 ribozyme.

### Embodiment 89

The mammalian cell of any one of claims 83-88, wherein said RNA decoy is a snoRNA.

### Embodiment 90

The mammalian cell of any one of claims 83-88, wherein said RNA decoy is a rev binding RNA decoy or a Tat binding RNA decoy

### Embodiment 91

The mammalian cell of any one of claims 81-90, wherein said viral entry inhibiting RNA is a cellular receptor siRNA.

### Embodiment 92

The mammalian cell of claim 91, wherein said cellular receptor siRNA is a T cell receptor siRNA.

### Embodiment 93

The mammalian cell of claim 92, wherein said T cell receptor siRNA is a small hairpin (sh) RNA.

### Embodiment 94

The mammalian cell of claim 93, wherein said shRNA is a CCR5 shRNA.

### Embodiment 95

The mammalian cell of claim 93, wherein said shRNA is a CXCR4 shRNA.

### Embodiment 96

The mammalian cell of any one of claims 81-90, wherein said viral entry inhibiting RNA is a nuclear receptor siRNA.

### Embodiment 97

The mammalian cell of claim 96, wherein said nuclear receptor siRNA is a transportin 3 (TNPO3) siRNA.

### Embodiment 98

The mammalian cell of any one of claims 81-97, further comprising an antiviral protein.

### Embodiment 99

The mammalian cell of claim 98, wherein said antiviral protein is a C46 fusion inhibitor.

### Embodiment 100

The mammalian cell of claim 98, wherein said antiviral protein is a mutant Rev protein.

### Embodiment 101

The mammalian cell of claim 100, wherein said mutant Rev protein is a Rev M10 protein.

### Embodiment 102

The mammalian cell of claim 81, wherein said first antiviral RNA is a Tat/Rev siRNA, said second antiviral RNA is a Rev siRNA, said third antiviral RNA is a Tat siRNA, and said viral entry inhibiting RNA is a CCR5 shRNA.

### Embodiment 103

The mammalian cell of claim 81, wherein said first antiviral RNA is a Tat/Rev siRNA, said second antiviral RNA is a Rev siRNA, said third antiviral RNA is a Tat binding RNA decoy, and said viral entry inhibiting RNA is a CCR5 shRNA.

### Embodiment 104

The mammalian cell of claim 81, wherein said first antiviral RNA is a Tat/Rev siRNA, said second antiviral RNA is a U5 ribozyme, said third antiviral RNA is a Tat binding RNA decoy, and said viral entry inhibiting RNA is a CCR5 shRNA.

### Embodiment 105

A kit comprising a recombinant antiviral polycistronic RNA comprising, (i) a first antiviral RNA, a second antiviral RNA and a third antiviral RNA; and (ii) a viral entry inhibiting RNA.

### Embodiment 106

The kit of claim 105, wherein said first antiviral RNA, said second antiviral RNA and said third antiviral RNA is a small interfering RNA (siRNA).

### Embodiment 107

The kit of claim 105 or 106, wherein said siRNA is a viral transcription inhibiting siRNA, a viral replication inhibiting siRNA, a viral transcription and replication inhibiting siRNA, a ribozyme or an RNA decoy.

### Embodiment 108

The kit of claim 107, wherein said viral transcription inhibiting siRNA is a Tat siRNA.

### Embodiment 109

The kit of any one of claims 105-108, wherein said viral replication inhibiting siRNA is a Rev siRNA.

### Embodiment 110

The kit of claim 107, wherein said viral transcription and replication inhibiting siRNA is a Tat/Rev siRNA.

### Embodiment 111

The kit of claim 107, wherein said ribozyme is a snoRNA.

### Embodiment 112

The kit of claim 107, wherein said ribozyme is a U5 ribozyme.

### Embodiment 113

The kit of claim 107, wherein said RNA decoy is a snoRNA.

### Embodiment 114

The kit of claim 107, wherein said RNA decoy is a rev binding RNA decoy or a Tat binding RNA decoy.

### Embodiment 115

The kit of claim 105, wherein said viral entry inhibiting RNA is a cellular receptor siRNA.

### Embodiment 116

The kit of claim 115, wherein said cellular receptor siRNA is a T cell receptor siRNA.

### Embodiment 117

The kit of claim 116, wherein said T cell receptor siRNA is a small hairpin (sh) RNA.

### Embodiment 118

The kit of claim 117, wherein said shRNA is a CCR5 shRNA.

### Embodiment 119

The kit of claim 117, wherein said shRNA is a CXCR4 shRNA.

### Embodiment 120

The kit of any one of claims 105-119, wherein said viral entry inhibiting RNA is a nuclear receptor siRNA.

### Embodiment 121

The kit of claim 120, wherein said nuclear receptor siRNA is a transportin 3 (TNPO3) siRNA.

### Embodiment 122

The kit of any one of claims 105-121, wherein said first antiviral RNA is a Tat/Rev siRNA, said second antiviral RNA is a Rev siRNA, said third antiviral RNA is a Tat siRNA, and said viral entry inhibiting RNA is a CCR5 shRNA.

### Embodiment 123

The kit of any one of claims 105-121, wherein said first antiviral RNA is a Tat/Rev siRNA, said second antiviral RNA is a Rev siRNA, said third antiviral RNA is a Tat binding RNA decoy, and said viral entry inhibiting RNA is a CCR5 shRNA.

### Embodiment 124

The kit of any one of claims 105-121, wherein said first antiviral RNA is a Tat/Rev siRNA, said second antiviral RNA is a U5 ribozyme, said third antiviral RNA is a Tat binding RNA decoy, and said viral entry inhibiting RNA is a CCR5 shRNA.

### Embodiment 125

A pharmaceutical composition comprising a pharmaceutically acceptable excipient and a recombinant viral particle comprising a recombinant nucleic acid of any one of claims 1-45.

### Embodiment 126

A method of treating an infectious disease in a subject in need thereof, said method comprising administering to said subject a therapeutically effective amount of a recombinant viral particle comprising a recombinant nucleic acid of any one of claims 1-45.

### Embodiment 127

The method of claim 126, wherein said infectious disease is caused by a virus.

### Embodiment 128

The method of claim 127, wherein said virus is HIV.

### Embodiment 129

The method of claim 126, wherein said subject suffers from AIDS.

### Embodiment 130

A method of inhibiting HIV replication in a patient, said method comprising administering to said patient a therapeutically effective amount of a recombinant viral particle comprising a recombinant nucleic acid of any one of claims 1-45, thereby inhibiting HIV replication in said patient.

1. A recombinant nucleic acid encoding an antiviral polycistronic RNA, said recombinant nucleic acid comprising a first RNA promoter operably linked to: (i) a first antiviral RNA encoding sequence, (ii) a second antiviral RNA encoding sequence and a (iii) third antiviral RNA encoding sequence, wherein said first RNA promoter is a forward promoter.

2. The recombinant nucleic acid of claim 1, further comprising a second RNA promoter operably linked to a viral entry inhibiting RNA encoding sequence, wherein said second RNA promoter is a reverse promoter.

3. (canceled)

4. (canceled)

5. The recombinant nucleic acid of claim 2, wherein said first RNA promoter is a RNA polymerase II promoter.

6. The recombinant nucleic acid of claim 5, wherein said RNA polymerase II promoter is a small nuclear RNA (snRNA) promoter.

7. (canceled)

8. The recombinant nucleic acid of claim 2, wherein said first antiviral RNA encoding sequence encodes a first small interfering RNA (siRNA), said second antiviral RNA encoding sequence encodes a second siRNA and said third antiviral RNA encoding sequence encodes a third siRNA.

9. The recombinant nucleic acid of claim 8, wherein said first siRNA, second siRNA and third siRNA are independently a viral transcription inhibiting siRNA, a viral replication inhibiting siRNA, a viral transcription and replication inhibiting siRNA, a ribozyme or an RNA decoy.

10.-16. (canceled)

17. The recombinant nucleic acid of claim 2, wherein said second RNA promoter is downstream of said third antiviral RNA encoding sequence.

18. The recombinant nucleic acid of claim 2, wherein said second RNA promoter is a polymerase III promoter.

19. The recombinant nucleic acid of claim 18, wherein said RNA polymerase III promoter is a small nuclear RNA (snRNA) promoter.

20. (canceled)

21. The recombinant nucleic acid of claim 2, wherein said viral entry inhibiting RNA encoding sequence encodes a cellular receptor siRNA.

22. The recombinant nucleic acid of claim 21, wherein said cellular receptor siRNA is a T cell receptor siRNA.

23. (canceled)

24. (canceled)

25. (canceled)

26. The recombinant nucleic acid of claim 2, wherein said viral entry inhibiting RNA encoding sequence encodes a nuclear receptor siRNA.

27. The recombinant nucleic acid of claim 26, wherein said nuclear receptor siRNA is a transportin 3 (TNPO3) siRNA.

28. (canceled)

29. The recombinant nucleic acid of claim 2, further comprising a transcriptional terminator sequence.

30. (canceled)

31. The recombinant nucleic acid of claim 29, wherein said transcriptional terminator sequence is downstream of said viral entry inhibiting RNA encoding sequence.

32. The recombinant nucleic acid of claim 1, further comprising a first nucleic acid linker connecting said first antiviral RNA encoding sequence to said second antiviral RNA encoding sequence and a second nucleic acid linker connecting said second antiviral RNA encoding sequence to said third antiviral RNA encoding sequence.

33. The recombinant nucleic acid of claim 32, wherein said first nucleic acid linker or said second nucleic acid linker comprise an intron sequence.

34. (canceled)

35. The recombinant nucleic acid of claim 2, further comprising an antiviral protein encoding sequence.

36. The recombinant nucleic acid of claim 35, wherein said antiviral protein encoding sequence is downstream of said viral entry inhibiting RNA encoding sequence.

37. (canceled)

38. (canceled)

39. (canceled)

40. The recombinant nucleic acid of claim 35, further comprising a transcriptional terminator sequence.

41. (canceled)

42. The recombinant nucleic acid of claim 40, wherein said transcriptional terminator sequence is downstream of said antiviral protein encoding sequence.

43. The recombinant nucleic acid of claim 2, wherein said first RNA promoter is a U1 promoter, said first antiviral RNA encoding sequence encodes a Tat/Rev siRNA, said second antiviral RNA encoding sequence encodes a Rev siRNA, said third antiviral RNA encoding sequence encodes a Tat siRNA, said second RNA promoter is a U6 promoter, and said viral entry inhibiting RNA encoding sequence encodes a CCR5 shRNA.

44. The recombinant nucleic acid of claim 2, wherein said first RNA promoter is a U1 promoter, said first antiviral RNA encoding sequence encodes a Tat/Rev siRNA, said second antiviral RNA encoding sequence encodes a Rev siRNA, said third antiviral RNA encoding sequence encodes a Tat binding RNA decoy, said second RNA promoter is a U6 promoter, and said viral entry inhibiting RNA encoding sequence encodes a CCR5 shRNA.

45. The recombinant nucleic acid of claim 2, wherein said first RNA promoter is a U1 promoter, said first antiviral RNA encoding sequence encodes a Tat/Rev siRNA, said second antiviral RNA encoding sequence encodes a U5 ribozyme, said third antiviral RNA encoding sequence encodes a Tat binding RNA decoy, said second RNA promoter is a U6 promoter, and said viral entry inhibiting RNA encoding sequence encodes a CCR5 shRNA.

46.-124. (canceled)

125. A pharmaceutical composition comprising a pharmaceutically acceptable excipient and a recombinant viral particle comprising a recombinant nucleic acid of claim 1.

126. A method of treating an infectious disease in a subject in need thereof, said method comprising administering to said subject a therapeutically effective amount of a recombinant viral particle comprising a recombinant nucleic acid of claim 1.

127.-130. (canceled)
